SEQUENCE LISTING (502 sequences)

SEQ ID NO: 1 - protein, 390 aa, Homo sapiens
MPPSGLRLLLLLLPLLWLLVLTPGRPAAGLSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 2 - protein, 112 aa, Homo sapiens
ALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 3 - protein, 414 aa, Homo sapiens
MHYCVLSAFLILHLVTVALSLSTCSTLDMDQFMRKRIEAIRGQILSKLKLTSPPEDYPEPEEVPPEVISIYNSTRDLLQEKASRRAAACERERSDEEYYAKEVYKIDMPPFFPSENAIPPTFYRPYFRIVRFDVSAMEKNASNLVKAEFRVFRLQNPKARVPEQRIELYQILKSKDLTSPTQRYIDSKVVKTRAEGEWLSFDVTDAVHEWLHHKDRNLGFKISLHCPCCTFVPSNNYIIPNKSEELEARFAGIDGTSTYTSGDQKTIKSTRKKNSGKTPHLLLMLLPSYRLESQQTNRRKKRALDAAYCFRNVQDNCCLRPLYIDFKRDLGWKWIHEPKGYNANFCAGACPYLWSSDTQHSRVLSLYNTINPEASASPCCVSQDLEPLTILYYIGKTPKIEQLSNMIVKSCKCS

SEQ ID NO: 4 - protein, 442 aa, Homo sapiens
MHYCVLSAFLILHLVTVALSLSTCSTLDMDQFMRKRIEAIRGQILSKLKLTSPPEDYPEPEEVPPEVISIYNSTRDLLQEKASRRAAACERERSDEEYYAKEVYKIDMPPFFPSETVCPVVTTPSGSVGSLCSRQSQVLCGYLDAIPPTFYRPYFRIVRFDVSAMEKNASNLVKAEFRVFRLQNPKARVPEQRIELYQILKSKDLTSPTQRYIDSKVVKTRAEGEWLSFDVTDAVHEWLHHKDRNLGFKISLHCPCCTFVPSNNYIIPNKSEELEARFAGIDGTSTYTSGDQKTIKSTRKKNSGKTPHLLLMLLPSYRLESQQTNRRKKRALDAAYCFRNVQDNCCLRPLYIDFKRDLGWKWIHEPKGYNANFCAGACPYLWSSDTQHSRVLSLYNTINPEASASPCCVSQDLEPLTILYYIGKTPKIEQLSNMIVKSCKCS

SEQ ID NO: 5 - protein, 112 aa, Homo sapiens
ALDAAYCFRNVQDNCCLRPLYIDFKRDLGWKWIHEPKGYNANFCAGACPYLWSSDTQHSRVLSLYNTINPEASASPCCVSQDLEPLTILYYIGKTPKIEQLSNMIVKSCKCS

SEQ ID NO: 6 - protein, 412 aa, Homo sapiens
MKMHLQRALVVLALLNFATVSLSLSTCTTLDFGHIKKKRVEAIRGQILSKLRLTSPPEPTVMTHVPYQVLALYNSTRELLEEMHGEREEGCTQENTESEYYAKEIHKFDMIQGLAEHNELAVCPKGITSKVFRFNVSSVEKNRTNLFRAEFRVLRVPNPSSKRNEQRIELFQILRPDEHIAKQRYIGGKNLPTRGTAEWLSFDVTDTVREWLLRRESNLGLEISIHCPCHTFQPNGDILENIHEVMEIKFKGVDNEDDHGRGDLGRLKKQKDHHNPHLILMMIPPHRLDNPGQGGQRKKRALDTNYCFRNLEENCCVRPLYIDFRQDLGWKWVHEPKGYYANFCSGPCPYLRSADTTHSTVLGLYNTLNPEASASPCCVPQDLEPLTILYYVGRTPKVEQLSNMVVKSCKCS

SEQ ID NO: 7 - protein, 309 aa, Homo sapiens
MKMHLQRALVVLALLNFATVSLSLSTCTTLDFGHIKKKRVEAIRGQILSKLRLTSPPEPTVMTHVPYQVLALYNSTRELLEEMHGEREEGCTQENTESEYYAKEIHKFDMIQGLAEHNELAVCPKGITSKVFRFNVSSVEKNRTNLFRAEFRVLRVPNPSSKRNEQRIELFQILRPDEHIAKQRYIGGKNLPTRGTAEWLSFDVTDTVREWLLRRESNLGLEISIHCPCHTFQPNGDILENIHEVMEIKFKGVDNEDDHGRGDLGRLKKQKDHHNPHLILMMIPPHRLDNPGQGGQRKKRALDTNYCFR

SEQ ID NO: 8 - protein, 112 aa, Homo sapiens [sequence omitted]

SEQ ID NO: 9 - protein, 426 aa, Homo sapiens
MPLLWLRGFLLASCWIIVRSSPTPGSEGHSAAPDCPSCALAALPKDVPNSQPEMVEAVKKHILNMLHLKKRPDVTQPVPKAALLNAIRKLHVGKVGENGYVEIEDDIGRRAEMNELMEQTSEIITFAESGTARKTLHFEISKEGSDLSVVERAEVWLFLKVPKANRTRTKVTIRLFQQQKHPQGSLDTGEEAEEVGLKGERSELLLSEKVVDARKSTWHVFPVSSSIQRLLDQGKSSLDVRIACEQCQESGASLVLLGKKKKKEEEGEGKKKGGGEGGAGADEEKEQSHRPFLMLQARQSEDHPHRRRRRGLECDGKVNICCKKQFFVSFKDIGWNDWIIAPSGYHANYCEGECPSHIAGTSGSSLSFHSTVINHYRMRGHSPFANLKSCCVPTKLRPMSMLYYDDGQNIIKKDIQNMIVEECGCS

SEQ ID NO: 10 - protein, 116 aa, Homo sapiens [sequence omitted]

SEQ ID NO: 11 - protein, 407 aa, Homo sapiens
MDGLPGRALGAACLLLLAAGWLGPEAWGSPTPPPTPAAPPPPPPPGSPGGSQDTCTSCGGFRRPEELGRVDGDFLEAVKRHILSRLQMRGRPNITHAVPKAAMVTALRKLHAGKVREDGRVEIPHLDGHASPGADGQERVSEIISFAETDGLASSRVRLYFFISNEGNQNLFVVQASLWLYLKLLPYVLEKGSRRKVRVKVYFQEQGHGDRWNMVEKRVDLKRSGWHTFPLTEAIQALFERGERRLNLDVQCDSCQELAVVPVFVDPGEESHRPFVVVQARLGDSRHRIRKRGLECDGRTNLCCRQQFFIDFRLIGWNDWIIAPTGYYGNYCEGSCPAYLAGVPGSASSFHTAVVNQYRMRGLNPGTVNSCCIPTKLSTMSMLYFDDEYNIVKRDVPNMIVEECGCA

SEQ ID NO: 12 - protein, 115 aa, Homo sapiens
GLECDGRTNLCCRQQFFIDFRLIGWNDWIIAPTGYYGNYCEGSCPAYLAGVPGSASSFHTAVVNQYRMRGLNPGTVNSCCIPTKLSTMSMLYFDDEYNIVKRDVPNMIVEECGCA

SEQ ID NO: 13 - protein, 10 aa, Artificial Sequence
  Description of Artificial Sequence Synthetic peptide
GLSLSNAKMG

SEQ ID NO: 14 - protein, 10 aa, Artificial Sequence
  Description of Artificial Sequence Synthetic peptide
GASVSNARTG

SEQ ID NO: 15 - protein, 8 aa, Artificial Sequence
  Description of Artificial Sequence Synthetic peptide
GFTASRNY

SEQ ID NO: 16 - protein, 8 aa, Artificial Sequence
  Description of Artificial Sequence Synthetic peptide
GFDFGEHW

SEQ ID NO: 17 - protein, 8 aa, Artificial Sequence
  Description of Artificial Sequence Synthetic peptide
GYTFSNYG

SEQ ID NO: 18 - protein, 10 aa, Artificial Sequence
  Description of Artificial Sequence Synthetic peptide
GLSLSNAKMG

SEQ ID NO: 19 - protein, 10 aa, Artificial Sequence
  Description of Artificial Sequence Synthetic peptide
GVSLSNARLG

SEQ ID NO: 20 - protein, 10 aa, Artificial Sequence
  Description of Artificial Sequence Synthetic peptide
GYSLTDARIG

SEQ ID NO: 21 - protein, 8 aa, Artificial Sequence
  Description of Artificial Sequence Synthetic peptide
GYTFSNYG

SEQ ID NO: 22 - protein, 8 aa, Artificial Sequence
  Description of Artificial Sequence Synthetic peptide
GFTLSNYP

SEQ ID NO: 23 - protein, 10 aa, Artificial Sequence
  Description of Artificial Sequence Synthetic peptide
GVSLSNARLG

SEQ ID NO: 24 - protein, 10 aa, Artificial Sequence
  Description of Artificial Sequence Synthetic peptide
GLSLSNAKMG

SEQ ID NO: 25 - protein, 8 aa, Artificial Sequence
  Description of Artificial Sequence Synthetic peptide
GFTASRNY

SEQ ID NO: 26 - protein, 10 aa, Artificial Sequence
  Description of Artificial Sequence Synthetic peptide
GYSLTDARIG

SEQ ID NO: 27 - protein, 10 aa, Artificial Sequence
  Description of Artificial Sequence Synthetic peptide
GFSLSTSGAT

SEQ ID NO: 28 - protein, 8 aa, Artificial Sequence
  Description of Artificial Sequence Synthetic peptide
GYTYTGYR

SEQ ID NO: 29 - protein, 10 aa, Artificial Sequence
  Description of Artificial Sequence Synthetic peptide
GVSLSDARMG

SEQ ID NO: 30 - protein, 10 aa, Artificial Sequence
  Description of Artificial Sequence Synthetic peptide
GFSLSTSGAT

SEQ ID NO: 31 - protein, 10 aa, Artificial Sequence
  Description of Artificial Sequence Synthetic peptide
GFTLSNPRMG

SEQ ID NO: 32 - protein, 10 aa, Artificial Sequence
  Description of Artificial Sequence Synthetic peptide
GFPLSTSRMG

SEQ ID NO: 33 - protein, 8 aa, Artificial Sequence
  Description of Artificial Sequence Synthetic peptide
GYTFPSHY

SEQ ID NO: 34 - protein, 10 aa, Artificial Sequence
  Description of Artificial Sequence Synthetic peptide
GFPLSTSRMG

SEQ ID NO: 35 - protein, 10 aa, Artificial Sequence
  Description of Artificial Sequence Synthetic peptide
GFPLSTSRMG

SEQ ID NO: 36 - protein, 10 aa, Artificial Sequence
  Description of Artificial Sequence Synthetic peptide
GFPLSTSRMG

SEQ ID NO: 37 - protein, 10 aa, Artificial Sequence
  Description of Artificial Sequence Synthetic peptide
GVSLSNVRMG

SEQ ID NO: 38 - protein, 8 aa, Artificial Sequence
  Description of Artificial Sequence Synthetic peptide
GYTFTSSA

SEQ ID NO: 39 - protein, 8 aa, Artificial Sequence
  Description of Artificial Sequence Synthetic peptide
GFTSSNHW

SEQ ID NO: 40 - protein, 8 aa, Artificial Sequence
  Description of Artificial Sequence Synthetic peptide
GFTFSDHH

SEQ ID NO: 41 - protein, 8 aa, Artificial Sequence
  Description of Artificial Sequence Synthetic peptide
GFTSSGYW

SEQ ID NO: 42 - protein, 8 aa, Artificial Sequence
  Description of Artificial Sequence Synthetic peptide
GFTSSNHW

SEQ ID NO: 43 - protein, 7 aa, Artificial Sequence
  Description of Artificial Sequence Synthetic peptide
GYPSPAT

SEQ ID NO: 44 - protein, 8 aa, Artificial Sequence
  Description of Artificial Sequence Synthetic peptide
GYTFTSYW

SEQ ID NO: 45 - protein, 8 aa, Artificial Sequence
  Description of Artificial Sequence Synthetic peptide
GDTSSHSH

SEQ ID NO: 46 - protein, 10 aa, Artificial Sequence
  Description of Artificial Sequence Synthetic peptide
GFSLSDTRWG

SEQ ID NO: 47 - protein, 8 aa, Artificial Sequence
  Description of Artificial Sequence Synthetic peptide
GYTFPSHY

SEQ ID NO: 48 - protein, 7 aa, Artificial Sequence
  Description of Artificial Sequence Synthetic peptide
VSTTGTT

SEQ ID NO: 49 - protein, 7 aa, Artificial Sequence
  Description of Artificial Sequence Synthetic peptide
INPSGRI

SEQ ID NO: 50 - protein, 8 aa, Artificial Sequence
  Description of Artificial Sequence Synthetic peptide
ISGSGDYT

SEQ ID NO: 51 - protein, 8 aa, Artificial Sequence
  Description of Artificial Sequence Synthetic peptide
IKGDASDT

SEQ ID NO: 52 - protein, 8 aa, Artificial Sequence
  Description of Artificial Sequence Synthetic peptide
ISKSGSSI

SEQ ID NO: 53 - protein, 7 aa, Artificial Sequence
  Description of Artificial Sequence Synthetic peptide
VSTTGTT

SEQ ID NO: 54 - protein, 7 aa, Artificial Sequence
  Description of Artificial Sequence Synthetic peptide
GSVTGST

SEQ ID NO: 55 - protein, 7 aa, Artificial Sequence
  Description of Artificial Sequence Synthetic peptide
INPSGRT

SEQ ID NO: 56 - protein, 8 aa, Artificial Sequence
  Description of Artificial Sequence Synthetic peptide
ISKSGSSI

SEQ ID NO: 57 - protein, 8 aa, Artificial Sequence
  Description of Artificial Sequence Synthetic peptide
TTSGSSTI

SEQ ID NO: 58 - protein, 7 aa, Artificial Sequence
  Description of Artificial Sequence Synthetic peptide
GSVTGST

SEQ ID NO: 59 - protein, 7 aa, Artificial Sequence
  Description of Artificial Sequence Synthetic peptide
VSTTGTT

SEQ ID NO: 60 - protein, 8 aa, Artificial Sequence
  Description of Artificial Sequence Synthetic peptide
ISGSGDYT

SEQ ID NO: 61 - protein, 7 aa, Artificial Sequence
  Description of Artificial Sequence Synthetic peptide
INPSGRT

SEQ ID NO: 62 - protein, 6 aa, Artificial Sequence
  Description of Artificial Sequence Synthetic peptide
ITSSGR

SEQ ID NO: 63 - protein, 8 aa, Artificial Sequence
  Description of Artificial Sequence Synthetic peptide
IDPKSGRT

SEQ ID NO: 64 - protein, 7 aa, Artificial Sequence
  Description of Artificial Sequence Synthetic peptide
VSTTGTT

SEQ ID NO: 65 - protein, 6 aa, Artificial Sequence
  Description of Artificial Sequence Synthetic peptide
ITSSGR

SEQ ID NO: 66 - protein, 7 aa, Artificial Sequence
  Description of Artificial Sequence Synthetic peptide
ISTSGRT

SEQ ID NO: 67 - protein, 7 aa, Artificial Sequence
  Description of Artificial Sequence Synthetic peptide
INPSGAT

SEQ ID NO: 68 - protein, 8 aa, Artificial Sequence
  Description of Artificial Sequence Synthetic peptide
MNPITGHT

SEQ ID NO: 69 - protein, 7 aa, Artificial Sequence
  Description of Artificial Sequence Synthetic peptide
INPSGAT

SEQ ID NO: 70 - protein, 7 aa, Artificial Sequence
  Description of Artificial Sequence Synthetic peptide
INPSGAT

SEQ ID NO: 71 - protein, 7 aa, Artificial Sequence
  Description of Artificial Sequence Synthetic peptide
INPSGAT

SEQ ID NO: 72 - protein, 7 aa, Artificial Sequence
  Description of Artificial Sequence Synthetic peptide
VNPSGST

SEQ ID NO: 73 - protein, 8 aa, Artificial Sequence
  Description of Artificial Sequence Synthetic peptide
IYPGSGHT

SEQ ID NO: 74 - protein, 8 aa, Artificial Sequence
  Description of Artificial Sequence Synthetic peptide
IGPKGLGT

SEQ ID NO: 75 - protein, 8 aa, Artificial Sequence
  Description of Artificial Sequence Synthetic peptide
ISPSGDFT

SEQ ID NO: 76 - protein, 7 aa, Artificial Sequence
  Description of Artificial Sequence Synthetic peptide
IGTAGVT

SEQ ID NO: 77 - protein, 8 aa, Artificial Sequence
  Description of Artificial Sequence Synthetic peptide
IGPKGLGT

SEQ ID NO: 78 - protein, 8 aa, Artificial Sequence
  Description of Artificial Sequence Synthetic peptide
MNPKSGYT

SEQ ID NO: 79 - protein, 8 aa, Artificial Sequence
  Description of Artificial Sequence Synthetic peptide
INPSNGGT

SEQ ID NO: 80 - protein, 8 aa, Artificial Sequence
  Description of Artificial Sequence Synthetic peptide
INPRSGAT

SEQ ID NO: 81 - protein, 7 aa, Artificial Sequence
  Description of Artificial Sequence Synthetic peptide
INDRGTT

SEQ ID NO: 82 - protein, 8 aa, Artificial Sequence
  Description of Artificial Sequence Synthetic peptide
MNPITGHT

SEQ ID NO: 83 - protein, 8 aa, Artificial Sequence
  Description of Artificial Sequence Synthetic peptide
ARAGYPDV

SEQ ID NO: 84 - protein, 8 aa, Artificial Sequence
  Description of Artificial Sequence Synthetic peptide
ARAGYPDV

SEQ ID NO: 85 - protein, 18 aa, Artificial Sequence
  Description of Artificial Sequence Synthetic peptide
ARSPRGPRLLGYYYGMDV

SEQ ID NO: 86 - protein, 13 aa, Artificial Sequence
  Description of Artificial Sequence Synthetic peptide
VKDLSKQWLGIDY

SEQ ID NO: 87 - protein, 16 aa, Artificial Sequence
  Description of Artificial Sequence Synthetic peptide
ARLPRLAWNYYYGMDV

SEQ ID NO: 88 - protein, 8 aa, Artificial Sequence
  Description of Artificial Sequence Synthetic peptide
ARAGYPDV

SEQ ID NO: 89 - protein, 13 aa, Artificial Sequence
  Description of Artificial Sequence Synthetic peptide
TTSYLVDTAMVTR

SEQ ID NO: 90 - protein, 8 aa, Artificial Sequence
  Description of Artificial Sequence Synthetic peptide
ARAGYPDV

SEQ ID NO: 91 - protein, 16 aa, Artificial Sequence
  Description of Artificial Sequence Synthetic peptide
ARLPRLAWNYYYGMDV

SEQ ID NO: 92 - protein, 16 aa, Artificial Sequence
  Description of Artificial Sequence Synthetic peptide
ARGPDYYAPLRSYMDV

SEQ ID NO: 93 - protein, 13 aa, Artificial Sequence
  Description of Artificial Sequence Synthetic peptide
TTSYLVDTAMVTR

SEQ ID NO: 94 - protein, 8 aa, Artificial Sequence
  Description of Artificial Sequence Synthetic peptide
ARAGYPDV

SEQ ID NO: 95 - protein, 18 aa, Artificial Sequence
  Description of Artificial Sequence Synthetic peptide
ARSPRGPRLLGYYYGMDV

SEQ ID NO: 96 - protein, 8 aa, Artificial Sequence
  Description of Artificial Sequence Synthetic peptide
ARAGYPDV

SEQ ID NO: 97 - protein, 13 aa, Artificial Sequence
  Description of Artificial Sequence Synthetic peptide
TTTKIVGATLRDY

SEQ ID NO: 98 - protein, 19 aa, Artificial Sequence
  Description of Artificial Sequence Synthetic peptide
AREVKQWLVPGRGVYWFDP

SEQ ID NO: 99 - protein, 8 aa, Artificial Sequence
  Description of Artificial Sequence Synthetic peptide
ARAGYPDV

SEQ ID NO: 100 - protein, 13 aa, Artificial Sequence
  Description of Artificial Sequence Synthetic peptide
TTTKIVGATLRDY

SEQ ID NO: 101 - protein, 8 aa, Artificial Sequence
  Description of Artificial Sequence Synthetic peptide
ARAGYPDV

SEQ ID NO: 102 - protein, 8 aa, Artificial Sequence
  Description of Artificial Sequence Synthetic peptide
ARAGYPDV

SEQ ID NO: 103 - protein, 20 aa, Artificial Sequence
  Description of Artificial Sequence Synthetic peptide
ARDMRYDILPGRSYYYGMDV

SEQ ID NO: 104 - protein, 8 aa, Artificial Sequence
  Description of Artificial Sequence Synthetic peptide
ARAGYPDV

SEQ ID NO: 105 - protein, 8 aa, Artificial Sequence
  Description of Artificial Sequence Synthetic peptide
ARAGYPDV

SEQ ID NO: 106 - protein, 8 aa, Artificial Sequence
  Description of Artificial Sequence Synthetic peptide
ARAGYPDV

SEQ ID NO: 107 - protein, 8 aa, Artificial Sequence
  Description of Artificial Sequence Synthetic peptide
ARAGYPDV

SEQ ID NO: 108 - protein, 11 aa, Artificial Sequence
  Description of Artificial Sequence Synthetic peptide
AREGDIFVMDV

SEQ ID NO: 109 - protein, 17 aa, Artificial Sequence
  Description of Artificial Sequence Synthetic peptide
ASIRDYGDKKGGSWFDP

SEQ ID NO: 110 - protein, 9 aa, Artificial Sequence
  Description of Artificial Sequence Synthetic peptide
ARDVSPVYY

SEQ ID NO: 111 - protein, 15 aa, Artificial Sequence
  Description of Artificial Sequence Synthetic peptide
ARGPHGPQQRLRFDP

SEQ ID NO: 112 - protein, 18 aa, Artificial Sequence
  Description of Artificial Sequence Synthetic peptide
ARDVRGWRALYYYYGMDV

SEQ ID NO: 113 - protein, 12 aa, Artificial Sequence
  Description of Artificial Sequence Synthetic peptide
ATLHGSGLAFDI

SEQ ID NO: 114 - protein, 13 aa, Artificial Sequence
  Description of Artificial Sequence Synthetic peptide
SITTVVAYWYFDV

SEQ ID NO: 115 - protein, 16 aa, Artificial Sequence
  Description of Artificial Sequence Synthetic peptide
ARDRGGRTNGVGAFDI

SEQ ID NO: 116 - protein, 14 aa, Artificial Sequence
  Description of Artificial Sequence Synthetic peptide
AHTLHTREFVGMDV

SEQ ID NO: 117 - protein, 20 aa, Artificial Sequence
  Description of Artificial Sequence Synthetic peptide
ARDMRYDILPGRSYYYGMDV

SEQ ID NO: 118 - protein, 6 aa, Artificial Sequence
  Description of Artificial Sequence Synthetic peptide
QNIGAA

SEQ ID NO: 119 - protein, 6 aa, Artificial Sequence
  Description of Artificial Sequence Synthetic peptide
QTVSSH

SEQ ID NO: 120 - protein, 11 aa, Artificial Sequence
  Description of Artificial Sequence Synthetic peptide
RSLVHGSGHNY

SEQ ID NO: 121 - protein, 11 aa, Artificial Sequence
  Description of Artificial Sequence Synthetic peptide
QSLLHSNEKNY

SEQ ID NO: 122 - protein, 11 aa, Artificial Sequence
  Description of Artificial Sequence Synthetic peptide
RSLVHGSGDNY

SEQ ID NO: 123 - protein, 6 aa, Artificial Sequence
  Description of Artificial Sequence Synthetic peptide
QNVYTN

SEQ ID NO: 124 - protein, 6 aa, Artificial Sequence
  Description of Artificial Sequence Synthetic peptide
QSLGTN

SEQ ID NO: 125 - protein, 6 aa, Artificial Sequence
  Description of Artificial Sequence Synthetic peptide
QTISRS

SEQ ID NO: 126 - protein, 11 aa, Artificial Sequence
  Description of Artificial Sequence Synthetic peptide
QSLLHSNEKNY

SEQ ID NO: 127 - protein, 11 aa, Artificial Sequence
  Description of Artificial Sequence Synthetic peptide
QSLQQSEGRTY

SEQ ID NO: 128 - protein, 6 aa, Artificial Sequence
  Description of Artificial Sequence Synthetic peptide
QRIGSD

SEQ ID NO: 129 - protein, 6 aa, Artificial Sequence
  Description of Artificial Sequence Synthetic peptide
QGISRD

SEQ ID NO: 130 - protein, 11 aa, Artificial Sequence
  Description of Artificial Sequence Synthetic peptide
QSLLHSNEKNY

SEQ ID NO: 131 - protein, 6 aa, Artificial Sequence
  Description of Artificial Sequence Synthetic peptide
QSIGTK

SEQ ID NO: 132 - protein, 12 aa, Artificial Sequence
  Description of Artificial Sequence Synthetic peptide
QSVLDTSTNRNY

SEQ ID NO: 133 - protein, 6 aa, Artificial Sequence
  Description of Artificial Sequence Synthetic peptide
NIGLRS

SEQ ID NO: 134 - protein, 6 aa, Artificial Sequence
  Description of Artificial Sequence Synthetic peptide
ESVRSN

SEQ ID NO: 135 - protein, 6 aa, Artificial Sequence
  Description of Artificial Sequence Synthetic peptide
QGISND

SEQ ID NO: 136 - protein, 6 aa, Artificial Sequence
  Description of Artificial Sequence Synthetic peptide
QGIGID

SEQ ID NO: 137 - protein, 6 aa, Artificial Sequence
  Description of Artificial Sequence Synthetic peptide
QSVSTH

SEQ ID NO: 138 - protein, 6 aa, Artificial Sequence
  Description of Artificial Sequence Synthetic peptide
DIRGMS

SEQ ID NO: 139 - protein, 6 aa, Artificial Sequence
  Description of Artificial Sequence Synthetic peptide
QNIGHS

SEQ ID NO: 140 - protein, 6 aa, Artificial Sequence
  Description of Artificial Sequence Synthetic peptide
RSVSSD

SEQ ID NO: 141 - protein, 6 aa, Artificial Sequence
  Description of Artificial Sequence Synthetic peptide
QSVRTS

SEQ ID NO: 142 - protein, 7 aa, Artificial Sequence
  Description of Artificial Sequence Synthetic peptide
QSISSSQ

SEQ ID NO: 143 - protein, 6 aa, Artificial Sequence
  Description of Artificial Sequence Synthetic peptide
MDIRND

SEQ ID NO: 144 - protein, 11 aa, Artificial Sequence
  Description of Artificial Sequence Synthetic peptide
QSLLHSSGYNF

SEQ ID NO: 145 - protein, 3 aa, Artificial Sequence
  Description of Artificial Sequence Synthetic peptide
QSL

SEQ ID NO: 146 - protein, 11 aa, Artificial Sequence
  Description of Artificial Sequence Synthetic peptide
QSLLSSYGYHN

SEQ ID NO: 147 - protein, 11 aa, Artificial Sequence
  Description of Artificial Sequence Synthetic peptide
QSLLHVTGYNY

SEQ ID NO: 148 - protein, 6 aa, Artificial Sequence
  Description of Artificial Sequence Synthetic peptide
QDIKSD

SEQ ID NO: 149 - protein, 5 aa, Artificial Sequence
  Description of Artificial Sequence Synthetic peptide
SSVSY

SEQ ID NO: 150 - protein, 9 aa, Artificial Sequence
  Description of Artificial Sequence Synthetic peptide
SGDIGRYNY

SEQ ID NO: 151 - protein, 12 aa, Artificial Sequence
  Description of Artificial Sequence Synthetic peptide
QSVLDTSTNRNY

SEQ ID NO: 152 - protein, 8 aa, Artificial Sequence
  Description of Artificial Sequence Synthetic peptide
TGSIASNY

SEQ ID NO: 153 - protein, 3 aa, Artificial Sequence
  Description of Artificial Sequence Synthetic peptide
GAS

SEQ ID NO: 154 - protein, 3 aa, Artificial Sequence
  Description of Artificial Sequence Synthetic peptide
DVS

SEQ ID NO: 155 - protein, 3 aa, Artificial Sequence
  Description of Artificial Sequence Synthetic peptide
GAS

SEQ ID NO: 156 - protein, 3 aa, Artificial Sequence
  Description of Artificial Sequence Synthetic peptide
DAS

SEQ ID NO: 157 - protein, 3 aa, Artificial Sequence
  Description of Artificial Sequence Synthetic peptide
MAS

SEQ ID NO: 158 - protein, 3 aa, Artificial Sequence
  Description of Artificial Sequence Synthetic peptide
GAS

SEQ ID NO: 159 - protein, 3 aa, Artificial Sequence
  Description of Artificial Sequence Synthetic peptide
GAS

SEQ ID NO: 160 - protein, 3 aa, Artificial Sequence
  Description of Artificial Sequence Synthetic peptide
DAS

SEQ ID NO: 161 - protein, 3 aa, Artificial Sequence
  Description of Artificial Sequence Synthetic peptide
DAS

SEQ ID NO: 162 - protein, 3 aa, Artificial Sequence
  Description of Artificial Sequence Synthetic peptide
DAS

SEQ ID NO: 163 - protein, 3 aa, Artificial Sequence
  Description of Artificial Sequence Synthetic peptide
SAS

SEQ ID NO: 164 - protein, 3 aa, Artificial Sequence
  Description of Artificial Sequence Synthetic peptide
AAS

SEQ ID NO: 165 - protein, 3 aa, Artificial Sequence
  Description of Artificial Sequence Synthetic peptide
DAS

SEQ ID NO: 166 - protein, 3 aa, Artificial Sequence
  Description of Artificial Sequence Synthetic peptide
DAS

SEQ ID NO: 167 - protein, 3 aa, Artificial Sequence
  Description of Artificial Sequence Synthetic peptide
GAS

SEQ ID NO: 168 - protein, 3 aa, Artificial Sequence
  Description of Artificial Sequence Synthetic peptide
DTN

SEQ ID NO: 169 - protein, 3 aa, Artificial Sequence
  Description of Artificial Sequence Synthetic peptide
GAS

SEQ ID NO: 170 - protein, 3 aa, Artificial Sequence
  Description of Artificial Sequence Synthetic peptide
DVS

SEQ ID NO: 171 - protein, 3 aa, Artificial Sequence
  Description of Artificial Sequence Synthetic peptide
DAS

SEQ ID NO: 172 - protein, 3 aa, Artificial Sequence
  Description of Artificial Sequence Synthetic peptide
DAT

SEQ ID NO: 173 - protein, 3 aa, Artificial Sequence
  Description of Artificial Sequence Synthetic peptide
EVN

SEQ ID NO: 174 - protein, 3 aa, Artificial Sequence
  Description of Artificial Sequence Synthetic peptide
GAS

SEQ ID NO: 175 - protein, 3 aa, Artificial Sequence
  Description of Artificial Sequence Synthetic peptide
DVS

SEQ ID NO: 176 - protein, 3 aa, Artificial Sequence
  Description of Artificial Sequence Synthetic peptide
DTS

SEQ ID NO: 177 - protein, 3 aa, Artificial Sequence
  Description of Artificial Sequence Synthetic peptide
GAS

SEQ ID NO: 178 - protein, 3 aa, Artificial Sequence
  Description of Artificial Sequence Synthetic peptide
DAS

SEQ ID NO: 179 - protein, 3 aa, Artificial Sequence
  Description of Artificial Sequence Synthetic peptide
MAT

SEQ ID NO: 180 - protein, 3 aa, Artificial Sequence
  Description of Artificial Sequence Synthetic peptide
MGS

SEQ ID NO: 181 - protein, 3 aa, Artificial Sequence
  Description of Artificial Sequence Synthetic peptide
MGS

SEQ ID NO: 182 - protein, 3 aa, Artificial Sequence
  Description of Artificial Sequence Synthetic peptide
GAS

SEQ ID NO: 183 - protein, 3 aa, Artificial Sequence
  Description of Artificial Sequence Synthetic peptide
RAS

SEQ ID NO: 184 - protein, 3 aa, Artificial Sequence
  Description of Artificial Sequence Synthetic peptide
RTY

SEQ ID NO: 185 - protein, 3 aa, Artificial Sequence
  Description of Artificial Sequence Synthetic peptide
EDI

SEQ ID NO: 186 - protein, 3 aa, Artificial Sequence
  Description of Artificial Sequence Synthetic peptide
GAS

SEQ ID NO: 187 - protein, 3 aa, Artificial Sequence
  Description of Artificial Sequence Synthetic peptide
DTT

SEQ ID NO: 188 - protein, 9 aa, Artificial Sequence
  Description of Artificial Sequence Synthetic peptide
QQYTAYPLT

SEQ ID NO: 189 - protein, 8 aa, Artificial Sequence
  Description of Artificial Sequence Synthetic peptide
QQYFTLPT

SEQ ID NO: 190 - protein, 9 aa, Artificial Sequence
  Description of Artificial Sequence Synthetic peptide
MQGTQAPRT

SEQ ID NO: 191 - protein, 9 aa, Artificial Sequence
  Description of Artificial Sequence Synthetic peptide
VQDLHTPFT

SEQ ID NO: 192 - protein, 9 aa, Artificial Sequence
  Description of Artificial Sequence Synthetic peptide
VQTLQTPST

SEQ ID NO: 193 - protein, 9 aa, Artificial Sequence
  Description of Artificial Sequence Synthetic peptide
LQYGSSPPL

SEQ ID NO: 194 - protein, 9 aa, Artificial Sequence
  Description of Artificial Sequence Synthetic peptide
QQYDTHPLS

SEQ ID NO: 195 - protein, 9 aa, Artificial Sequence
  Description of Artificial Sequence Synthetic peptide
QQYSSYPSL

SEQ ID NO: 196 - protein, 9 aa, Artificial Sequence
  Description of Artificial Sequence Synthetic peptide
VQDLHTPFT

SEQ ID NO: 197 - protein, 9 aa, Artificial Sequence
  Description of Artificial Sequence Synthetic peptide
MQVLRTPIT

SEQ ID NO: 198 - protein, 6 aa, Artificial Sequence
  Description of Artificial Sequence Synthetic peptide
STMAIP

SEQ ID NO: 199 - protein, 10 aa, Artificial Sequence
  Description of Artificial Sequence Synthetic peptide
QQYVSAPPYT

SEQ ID NO: 200 - protein, 9 aa, Artificial Sequence
  Description of Artificial Sequence Synthetic peptide
VQDLHTPFT

SEQ ID NO: 201 - protein, 11 aa, Artificial Sequence
  Description of Artificial Sequence Synthetic peptide
QQYGSSPPVYS

SEQ ID NO: 202 - protein, 9 aa, Artificial Sequence
  Description of Artificial Sequence Synthetic peptide
QQSYSHPPT

SEQ ID NO: 203 - protein, 10 aa, Artificial Sequence
  Description of Artificial Sequence Synthetic peptide
SSYTSSSTDV

SEQ ID NO: 204 - protein, 10 aa, Artificial Sequence
  Description of Artificial Sequence Synthetic peptide
QQYHTWPPWA

SEQ ID NO: 205 - protein, 8 aa, Artificial Sequence
  Description of Artificial Sequence Synthetic peptide
QQYDNPPS

SEQ ID NO: 206 - protein, 9 aa, Artificial Sequence
  Description of Artificial Sequence Synthetic peptide
RQYATFPQT

SEQ ID NO: 207 - protein, 9 aa, Artificial Sequence
  Description of Artificial Sequence Synthetic peptide
LQYTNYPWT

SEQ ID NO: 208 - protein, 11 aa, Artificial Sequence
  Description of Artificial Sequence Synthetic peptide
AAWDASVAGYV

SEQ ID NO: 209 - protein, 9 aa, Artificial Sequence
  Description of Artificial Sequence Synthetic peptide
QQYITWPST

SEQ ID NO: 210 - protein, 10 aa, Artificial Sequence
  Description of Artificial Sequence Synthetic peptide
QQYGSSLPPT

SEQ ID NO: 211 - protein, 7 aa, Artificial Sequence
  Description of Artificial Sequence Synthetic peptide
QQYGNWH

SEQ ID NO: 212 - protein, 9 aa, Artificial Sequence
  Description of Artificial Sequence Synthetic peptide
QQYNDRPRT

SEQ ID NO: 213 - protein, 9 aa, Artificial Sequence
  Description of Artificial Sequence Synthetic peptide
QQAKSFPPT

SEQ ID NO: 214 - protein, 9 aa, Artificial Sequence
  Description of Artificial Sequence Synthetic peptide
VQDLHTLPQ

SEQ ID NO: 215 - protein, 5 aa, Artificial Sequence
  Description of Artificial Sequence Synthetic peptide
QSGVT

SEQ ID NO: 216 - protein, 9 aa, Artificial Sequence
  Description of Artificial Sequence Synthetic peptide
MQGTQAPRT

SEQ ID NO: 217 - protein, 9 aa, Artificial Sequence
  Description of Artificial Sequence Synthetic peptide
MQSTQWPHT

SEQ ID NO: 218 - protein, 9 aa, Artificial Sequence
  Description of Artificial Sequence Synthetic peptide
QQYRSFPWS

SEQ ID NO: 219 - protein, 9 aa, Artificial Sequence
  Description of Artificial Sequence Synthetic peptide
QQRSSYPWT

SEQ ID NO: 220 - protein, 10 aa, Artificial Sequence
  Description of Artificial Sequence Synthetic peptide
QVWDATLDLV

SEQ ID NO: 221 - protein, 9 aa, Artificial Sequence
  Description of Artificial Sequence Synthetic peptide
QQYETWPWT

SEQ ID NO: 222 - protein, 11 aa, Artificial Sequence
  Description of Artificial Sequence Synthetic peptide
QSTERGRSDVI

SEQ ID NO: 223 - protein, 7 aa, Artificial Sequence
  Description of Artificial Sequence Synthetic peptide
NAKMGVN

SEQ ID NO: 224 - protein, 7 aa, Artificial Sequence
  Description of Artificial Sequence Synthetic peptide
NARTGVN

SEQ ID NO: 225 - protein, 5 aa, Artificial Sequence
  Description of Artificial Sequence Synthetic peptide
RNYMS

SEQ ID NO: 226 - protein, 5 aa, Artificial Sequence
  Description of Artificial Sequence Synthetic peptide
EHWMS

SEQ ID NO: 227 - protein, 5 aa, Artificial Sequence
  Description of Artificial Sequence Synthetic peptide
NYGMS

SEQ ID NO: 228 - protein, 7 aa, Artificial Sequence
  Description of Artificial Sequence Synthetic peptide
NAKMGVN

SEQ ID NO: 229 - protein, 7 aa, Artificial Sequence
  Description of Artificial Sequence Synthetic peptide
NARLGVN

SEQ ID NO: 230 - protein, 7 aa, Artificial Sequence
  Description of Artificial Sequence Synthetic peptide
DARIGVN

SEQ ID NO: 231 - protein, 5 aa, Artificial Sequence
  Description of Artificial Sequence Synthetic peptide
NYGMS

SEQ ID NO: 232 - protein, 5 aa, Artificial Sequence
  Description of Artificial Sequence Synthetic peptide
NYPMS

SEQ ID NO: 233 - protein, 7 aa, Artificial Sequence
  Description of Artificial Sequence Synthetic peptide
NARLGVN

SEQ ID NO: 234 - protein, 7 aa, Artificial Sequence
  Description of Artificial Sequence Synthetic peptide
NAKMGVN

SEQ ID NO: 235 - protein, 5 aa, Artificial Sequence
  Description of Artificial Sequence Synthetic peptide
RNYMS

SEQ ID NO: 236 - protein, 7 aa, Artificial Sequence
  Description of Artificial Sequence Synthetic peptide
DARIGVN

SEQ ID NO: 237 - protein, 7 aa, Artificial Sequence
  Description of Artificial Sequence Synthetic peptide
TSGATVN

SEQ ID NO: 238 - protein, 5 aa, Artificial Sequence
  Description of Artificial Sequence Synthetic peptide
GYRIS

SEQ ID NO: 239 - protein, 7 aa, Artificial Sequence
  Description of Artificial Sequence Synthetic peptide
DARMGVN

SEQ ID NO: 240 - protein, 7 aa, Artificial Sequence
  Description of Artificial Sequence Synthetic peptide
TSGATVN

SEQ ID NO: 241 - protein, 7 aa, Artificial Sequence
  Description of Artificial Sequence Synthetic peptide
NPRMGVN

SEQ ID NO: 242 - protein, 7 aa, Artificial Sequence
  Description of Artificial Sequence Synthetic peptide
TSRMGVN

SEQ ID NO: 243 - protein, 5 aa, Artificial Sequence
  Description of Artificial Sequence Synthetic peptide
SHYIS

SEQ ID NO: 244 - protein, 7 aa, Artificial Sequence
  Description of Artificial Sequence Synthetic peptide
TSRMGVN

SEQ ID NO: 245 - protein, 7 aa, Artificial Sequence
  Description of Artificial Sequence Synthetic peptide
TSRMGVN

SEQ ID NO: 246 - protein, 7 aa, Artificial Sequence
  Description of Artificial Sequence Synthetic peptide
TSRMGVN

SEQ ID NO: 247 - protein, 7 aa, Artificial Sequence
  Description of Artificial Sequence Synthetic peptide
NVRMGVN

SEQ ID NO: 248 - protein, 5 aa, Artificial Sequence
  Description of Artificial Sequence Synthetic peptide
SSAIS

SEQ ID NO: 249 - protein, 5 aa, Artificial Sequence
  Description of Artificial Sequence Synthetic peptide
NHWMS

SEQ ID NO: 250 - protein, 5 aa, Artificial Sequence
  Description of Artificial Sequence Synthetic peptide
DHHMS

SEQ ID NO: 251 - protein, 5 aa, Artificial Sequence
  Description of Artificial Sequence Synthetic peptide
GYWMS

SEQ ID NO: 252 - protein, 5 aa, Artificial Sequence
  Description of Artificial Sequence Synthetic peptide
NHWMS

SEQ ID NO: 253 - protein, 4 aa, Artificial Sequence
  Description of Artificial Sequence Synthetic peptide
ATIS

SEQ ID NO: 254 - protein, 5 aa, Artificial Sequence
  Description of Artificial Sequence Synthetic peptide
SYWMH

SEQ ID NO: 255 - protein, 5 aa, Artificial Sequence
  Description of Artificial Sequence Synthetic peptide
HSHIH

SEQ ID NO: 256 - protein, 7 aa, Artificial Sequence
  Description of Artificial Sequence Synthetic peptide
DTRWGVN

SEQ ID NO: 257 - protein, 5 aa, Artificial Sequence
  Description of Artificial Sequence Synthetic peptide
SHYIS

SEQ ID NO: 258 - protein, 16 aa, Artificial Sequence
  Description of Artificial Sequence Synthetic peptide
YVSTTGTTDYNPSLKS

SEQ ID NO: 259 - protein, 16 aa, Artificial Sequence
  Description of Artificial Sequence Synthetic peptide
YINPSGRIDYNPSLKS

SEQ ID NO: 260 - protein, 17 aa, Artificial Sequence
  Description of Artificial Sequence Synthetic peptide
SISGSGDYTYYPDSVKG

SEQ ID NO: 261 - protein, 17 aa, Artificial Sequence
  Description of Artificial Sequence Synthetic peptide
SIKGDASDTYYPDSVKG

SEQ ID NO: 262 - protein, 17 aa, Artificial Sequence
  Description of Artificial Sequence Synthetic peptide
SISKSGSSIYYPDSVKG

SEQ ID NO: 263 - protein, 16 aa, Artificial Sequence
  Description of Artificial Sequence Synthetic peptide
YVSTTGTTDYNPSLKS

SEQ ID NO: 264 - protein, 16 aa, Artificial Sequence
  Description of Artificial Sequence Synthetic peptide
YGSVTGSTDYNPSLKS

SEQ ID NO: 265 - protein, 16 aa, Artificial Sequence
  Description of Artificial Sequence Synthetic peptide
YINPSGRTDYNPSLKS

SEQ ID NO: 266 - protein, 17 aa, Artificial Sequence
  Description of Artificial Sequence Synthetic peptide
SISKSGSSIYYPDSVKG

SEQ ID NO: 267 - protein, 17 aa, Artificial Sequence
  Description of Artificial Sequence Synthetic peptide
STTSGSSTIYYPDSVKG

SEQ ID NO: 268 - protein, 16 aa, Artificial Sequence
  Description of Artificial Sequence Synthetic peptide
YGSVTGSTDYNPSLKS

SEQ ID NO: 269 - protein, 16 aa, Artificial Sequence
  Description of Artificial Sequence Synthetic peptide
YVSTTGTTDYNPSLKS

SEQ ID NO: 270 - protein, 17 aa, Artificial Sequence
  Description of Artificial Sequence Synthetic peptide
SISGSGDYTYYPDSVKG

SEQ ID NO: 271 - protein, 16 aa, Artificial Sequence
  Description of Artificial Sequence Synthetic peptide
YINPSGRTDYNPSLKS

SEQ ID NO: 272 - protein, 16 aa, Artificial Sequence
  Description of Artificial Sequence Synthetic peptide
YITSSGRTVYNSALKS

SEQ ID NO: 273 - protein, 17 aa, Artificial Sequence
  Description of Artificial Sequence Synthetic peptide
WIDPKSGRTNYAQKLQG

SEQ ID NO: 274 - protein, 16 aa, Artificial Sequence
  Description of Artificial Sequence Synthetic peptide
YVSTTGTTDYNPSLKS

SEQ ID NO: 275 - protein, 16 aa, Artificial Sequence
  Description of Artificial Sequence Synthetic peptide
YITSSGRTVYNSALKS

SEQ ID NO: 276 - protein, 16 aa, Artificial Sequence
  Description of Artificial Sequence Synthetic peptide
YISTSGRTDYNPSLKS

SEQ ID NO: 277 - protein, 16 aa, Artificial Sequence
  Description of Artificial Sequence Synthetic peptide
YINPSGATDYNPSLKS

SEQ ID NO: 278 - protein, 17 aa, Artificial Sequence
  Description of Artificial Sequence Synthetic peptide
GMNPITGHTIYAQKLQG

SEQ ID NO: 279 - protein, 16 aa, Artificial Sequence
  Description of Artificial Sequence Synthetic peptide
YINPSGATDYNPSLKS

SEQ ID NO: 280 - protein, 16 aa, Artificial Sequence
  Description of Artificial Sequence Synthetic peptide
YINPSGATDYNPSLKS

SEQ ID NO: 281 - protein, 16 aa, Artificial Sequence
  Description of Artificial Sequence Synthetic peptide
YINPSGATDYNPSLKS

SEQ ID NO: 282 - protein, 16 aa, Artificial Sequence
  Description of Artificial Sequence Synthetic peptide
YVNPSGSTDYNPSLKS

SEQ ID NO: 283 - protein, 17 aa, Artificial Sequence
  Description of Artificial Sequence Synthetic peptide
GIYPGSGHTIYAQKFQG

SEQ ID NO: 284 - protein, 17 aa, Artificial Sequence
  Description of Artificial Sequence Synthetic peptide
SIGPKGLGTYYPDSVKG

SEQ ID NO: 285 - protein, 17 aa, Artificial Sequence
  Description of Artificial Sequence Synthetic peptide
SISPSGDFTYYPDSVKG

SEQ ID NO: 286 - protein, 16 aa, Artificial Sequence
  Description of Artificial Sequence Synthetic peptide
SIGTAGVTYYPDSVKG

SEQ ID NO: 287 - protein, 17 aa, Artificial Sequence
  Description of Artificial Sequence Synthetic peptide
SIGPKGLGTYYPDSVKG

SEQ ID NO: 288 - protein, 17 aa, Artificial Sequence
  Description of Artificial Sequence Synthetic peptide
WMNPKSGYTNYAQKFQG

SEQ ID NO: 289 - protein, 17 aa, Artificial Sequence
  Description of Artificial Sequence Synthetic peptide
NINPSNGGTNYNEKFKS

SEQ ID NO: 290 - protein, 17 aa, Artificial Sequence
  Description of Artificial Sequence Synthetic peptide
WINPRSGATNYAQKLQG

SEQ ID NO: 291 - protein, 16 aa, Artificial Sequence
  Description of Artificial Sequence Synthetic peptide
YINDRGTTDYNSALKS

SEQ ID NO: 292 - protein, 17 aa, Artificial Sequence
  Description of Artificial Sequence Synthetic peptide
GMNPITGHTIYAQKLQG

SEQ ID NO: 293 - protein, 6 aa, Artificial Sequence
  Description of Artificial Sequence Synthetic peptide
AGYPDV

SEQ ID NO: 294 - protein, 6 aa, Artificial Sequence
  Description of Artificial Sequence Synthetic peptide
AGYPDV

SEQ ID NO: 295 - protein, 16 aa, Artificial Sequence
  Description of Artificial Sequence Synthetic peptide
SPRGPRLLGYYYGMDV

SEQ ID NO: 296 - protein, 11 aa, Artificial Sequence
  Description of Artificial Sequence Synthetic peptide
DLSKQWLGIDY

SEQ ID NO: 297 - protein, 14 aa, Artificial Sequence
  Description of Artificial Sequence Synthetic peptide
LPRLAWNYYYGMDV

SEQ ID NO: 298 - protein, 6 aa, Artificial Sequence
  Description of Artificial Sequence Synthetic peptide
AGYPDV

SEQ ID NO: 299 - protein, 11 aa, Artificial Sequence
  Description of Artificial Sequence Synthetic peptide
SYLVDTAMVTR

SEQ ID NO: 300 - protein, 6 aa, Artificial Sequence
  Description of Artificial Sequence Synthetic peptide
AGYPDV

SEQ ID NO: 301 - protein, 14 aa, Artificial Sequence
  Description of Artificial Sequence Synthetic peptide
LPRLAWNYYYGMDV

SEQ ID NO: 302 - protein, 14 aa, Artificial Sequence
  Description of Artificial Sequence Synthetic peptide
GPDYYAPLRSYMDV

SEQ ID NO: 303 - protein, 11 aa, Artificial Sequence
  Description of Artificial Sequence Synthetic peptide
SYLVDTAMVTR

SEQ ID NO: 304 - protein, 6 aa, Artificial Sequence
  Description of Artificial Sequence Synthetic peptide
AGYPDV

SEQ ID NO: 305 - protein, 16 aa, Artificial Sequence
  Description of Artificial Sequence Synthetic peptide
SPRGPRLLGYYYGMDV

SEQ ID NO: 306 - protein, 6 aa, Artificial Sequence
  Description of Artificial Sequence Synthetic peptide
AGYPDV

SEQ ID NO: 307 - protein, 11 aa, Artificial Sequence
  Description of Artificial Sequence Synthetic peptide
TKIVGATLRDY

SEQ ID NO: 308 - protein, 17 aa, Artificial Sequence
  Description of Artificial Sequence Synthetic peptide
EVKQWLVPGRGVYWFDP

SEQ ID NO: 309 - protein, 6 aa, Artificial Sequence
  Description of Artificial Sequence Synthetic peptide
AGYPDV

SEQ ID NO: 310 - protein, 11 aa, Artificial Sequence
  Description of Artificial Sequence Synthetic peptide
TKIVGATLRDY

SEQ ID NO: 311 - protein, 6 aa, Artificial Sequence
  Description of Artificial Sequence Synthetic peptide
AGYPDV

SEQ ID NO: 312 - protein, 6 aa, Artificial Sequence
  Description of Artificial Sequence Synthetic peptide
AGYPDV

SEQ ID NO: 313 - protein, 18 aa, Artificial Sequence
  Description of Artificial Sequence Synthetic peptide
DMRYDILPGRSYYYGMDV

SEQ ID NO: 314 - protein, 6 aa, Artificial Sequence
  Description of Artificial Sequence Synthetic peptide
AGYPDV

SEQ ID NO: 315 - protein, 6 aa, Artificial Sequence
  Description of Artificial Sequence Synthetic peptide
AGYPDV

SEQ ID NO: 316 - protein, 6 aa, Artificial Sequence
  Description of Artificial Sequence Synthetic peptide
AGYPDV

SEQ ID NO: 317 - protein, 6 aa, Artificial Sequence
  Description of Artificial Sequence Synthetic peptide
AGYPDV

SEQ ID NO: 318 - protein, 9 aa, Artificial Sequence
  Description of Artificial Sequence Synthetic peptide
EGDIFVMDV

SEQ ID NO: 319 - protein, 15 aa, Artificial Sequence
  Description of Artificial Sequence Synthetic peptide
IRDYGDKKGGSWFDP

SEQ ID NO: 320 - protein, 7 aa, Artificial Sequence
  Description of Artificial Sequence Synthetic peptide
DVSPVYY

SEQ ID NO: 321 - protein, 13 aa, Artificial Sequence
  Description of Artificial Sequence Synthetic peptide
GPHGPQQRLRFDP

SEQ ID NO: 322 - protein, 16 aa, Artificial Sequence
  Description of Artificial Sequence Synthetic peptide
DVRGWRALYYYYGMDV

SEQ ID NO: 323 - protein, 10 aa, Artificial Sequence
  Description of Artificial Sequence Synthetic peptide
LHGSGLAFDI

SEQ ID NO: 324 - protein, 11 aa, Artificial Sequence
  Description of Artificial Sequence Synthetic peptide
TTVVAYWYFDV

SEQ ID NO: 325 - protein, 14 aa, Artificial Sequence
  Description of Artificial Sequence Synthetic peptide
DRGGRTNGVGAFDI

SEQ ID NO: 326 - protein, 12 aa, Artificial Sequence
  Description of Artificial Sequence Synthetic peptide
TLHTREFVGMDV

SEQ ID NO: 327 - protein, 18 aa, Artificial Sequence
  Description of Artificial Sequence Synthetic peptide
DMRYDILPGRSYYYGMDV

SEQ ID NO: 328 - protein, 11 aa, Artificial Sequence
  Description of Artificial Sequence Synthetic peptide
RASQNIGAALA

SEQ ID NO: 329 - protein, 11 aa, Artificial Sequence
  Description of Artificial Sequence Synthetic peptide
RASQTVSSHLA

SEQ ID NO: 330 - protein, 16 aa, Artificial Sequence
  Description of Artificial Sequence Synthetic peptide
RSSRSLVHGSGHNYLH

SEQ ID NO: 331 - protein, 16 aa, Artificial Sequence
  Description of Artificial Sequence Synthetic peptide
RSSQSLLHSNEKNYLH

SEQ ID NO: 332 - protein, 16 aa, Artificial Sequence
  Description of Artificial Sequence Synthetic peptide
RSSRSLVHGSGDNYLH

SEQ ID NO: 333 - protein, 11 aa, Artificial Sequence
  Description of Artificial Sequence Synthetic peptide
RASQNVYTNLA

SEQ ID NO: 334 - protein, 11 aa, Artificial Sequence
  Description of Artificial Sequence Synthetic peptide
RASQSLGTNLA

SEQ ID NO: 335 - protein, 11 aa, Artificial Sequence
  Description of Artificial Sequence Synthetic peptide
RASQTISRSLA

SEQ ID NO: 336 - protein, 16 aa, Artificial Sequence
  Description of Artificial Sequence Synthetic peptide
RSSQSLLHSNEKNYLH

SEQ ID NO: 337 - protein, 16 aa, Artificial Sequence
  Description of Artificial Sequence Synthetic peptide
RSSQSLQQSEGRTYLH

SEQ ID NO: 338 - protein, 11 aa, Artificial Sequence
  Description of Artificial Sequence Synthetic peptide
RASQRIGSDLA

SEQ ID NO: 339 - protein, 11 aa, Artificial Sequence
  Description of Artificial Sequence Synthetic peptide
RASQGISRDLA

SEQ ID NO: 340 - protein, 16 aa, Artificial Sequence
  Description of Artificial Sequence Synthetic peptide
RSSQSLLHSNEKNYLH

SEQ ID NO: 341 - protein, 11 aa, Artificial Sequence
  Description of Artificial Sequence Synthetic peptide
RASQSIGTKLA

SEQ ID NO: 342 - protein, 17 aa, Artificial Sequence
  Description of Artificial Sequence Synthetic peptide
KSSQSVLDTSTNRNYLA

SEQ ID NO: 343 - protein, 11 aa, Artificial Sequence
  Description of Artificial Sequence Synthetic peptide
TGTNIGLRSVS

SEQ ID NO: 344 - protein, 11 aa, Artificial Sequence
  Description of Artificial Sequence Synthetic peptide
RASESVRSNLA

SEQ ID NO: 345 - protein, 11 aa, Artificial Sequence
  Description of Artificial Sequence Synthetic peptide
RASQGISNDLA

SEQ ID NO: 346 - protein, 11 aa, Artificial Sequence
  Description of Artificial Sequence Synthetic peptide
RASQGIGIDLA

SEQ ID NO: 347 - protein, 11 aa, Artificial Sequence
  Description of Artificial Sequence Synthetic peptide
RASQSVSTHLA

SEQ ID NO: 348 - protein, 11 aa, Artificial Sequence
  Description of Artificial Sequence Synthetic peptide
TGTDIRGMSVS

SEQ ID NO: 349 - protein, 11 aa, Artificial Sequence
  Description of Artificial Sequence Synthetic peptide
RASQNIGHSLA

SEQ ID NO: 350 - protein, 11 aa, Artificial Sequence
  Description of Artificial Sequence Synthetic peptide
RASRSVSSDLA

SEQ ID NO: 351 - protein, 11 aa, Artificial Sequence
  Description of Artificial Sequence Synthetic peptide
RASQSVRTSLA

SEQ ID NO: 352 - protein, 12 aa, Artificial Sequence
  Description of Artificial Sequence Synthetic peptide
RASQSISSSQLA

SEQ ID NO: 353 - protein, 11 aa, Artificial Sequence
  Description of Artificial Sequence Synthetic peptide
RASMDIRNDLA

SEQ ID NO: 354 - protein, 16 aa, Artificial Sequence
  Description of Artificial Sequence Synthetic peptide
RSSQSLLHSSGYNFLH

SEQ ID NO: 355 - protein, 8 aa, Artificial Sequence
  Description of Artificial Sequence Synthetic peptide
RSSQSLLH

SEQ ID NO: 356 - protein, 16 aa, Artificial Sequence
  Description of Artificial Sequence Synthetic peptide
RSSQSLLSSYGYHNLH

SEQ ID NO: 357 - protein, 16 aa, Artificial Sequence
  Description of Artificial Sequence Synthetic peptide
RSSQSLLHVTGYNYLH

SEQ ID NO: 358 - protein, 11 aa, Artificial Sequence
  Description of Artificial Sequence Synthetic peptide
RASQDIKSDLA

SEQ ID NO: 359 - protein, 10 aa, Artificial Sequence
  Description of Artificial Sequence Synthetic peptide
SASSSVSYMY

SEQ ID NO: 360 - protein, 14 aa, Artificial Sequence
  Description of Artificial Sequence Synthetic peptide
TGTSGDIGRYNYVS

SEQ ID NO: 361 - protein, 17 aa, Artificial Sequence
  Description of Artificial Sequence Synthetic peptide
KSSQSVLDTSTNRNYLA

SEQ ID NO: 362 - protein, 13 aa, Artificial Sequence
  Description of Artificial Sequence Synthetic peptide
TGTTGSIASNYVS

SEQ ID NO: 363 - protein, 7 aa, Artificial Sequence
  Description of Artificial Sequence Synthetic peptide
GASTREY

SEQ ID NO: 364 - protein, 7 aa, Artificial Sequence
  Description of Artificial Sequence Synthetic peptide
DVSHRAS

SEQ ID NO: 365 - protein, 7 aa, Artificial Sequence
  Description of Artificial Sequence Synthetic peptide
GASTRVA

SEQ ID NO: 366 - protein, 7 aa, Artificial Sequence
  Description of Artificial Sequence Synthetic peptide
DASKRAS

SEQ ID NO: 367 - protein, 7 aa, Artificial Sequence
  Description of Artificial Sequence Synthetic peptide
MASNRAP

SEQ ID NO: 368 - protein, 7 aa, Artificial Sequence
  Description of Artificial Sequence Synthetic peptide
GASARAS

SEQ ID NO: 369 - protein, 7 aa, Artificial Sequence
  Description of Artificial Sequence Synthetic peptide
GASSRPS

SEQ ID NO: 370 - protein, 7 aa, Artificial Sequence
  Description of Artificial Sequence Synthetic peptide
DASSLAG

SEQ ID NO: 371 - protein, 7 aa, Artificial Sequence
  Description of Artificial Sequence Synthetic peptide
DASKRAS

SEQ ID NO: 372 - protein, 7 aa, Artificial Sequence
  Description of Artificial Sequence Synthetic peptide
DASKRAS

SEQ ID NO: 373 - protein, 7 aa, Artificial Sequence
  Description of Artificial Sequence Synthetic peptide
SASSRAP

SEQ ID NO: 374 - protein, 7 aa, Artificial Sequence
  Description of Artificial Sequence Synthetic peptide
AASTLVS

SEQ ID NO: 375 - protein, 7 aa, Artificial Sequence
  Description of Artificial Sequence Synthetic peptide
DASKRAS

SEQ ID NO: 376 - protein, 7 aa, Artificial Sequence
  Description of Artificial Sequence Synthetic peptide
DASSRVP

SEQ ID NO: 377 - protein, 7 aa, Artificial Sequence
  Description of Artificial Sequence Synthetic peptide
GASIRAG

SEQ ID NO: 378 - protein, 7 aa, Artificial Sequence
  Description of Artificial Sequence Synthetic peptide
DTNIRSS

SEQ ID NO: 379 - protein, 7 aa, Artificial Sequence
  Description of Artificial Sequence Synthetic peptide
GASNRAS

SEQ ID NO: 380 - protein, 7 aa, Artificial Sequence
  Description of Artificial Sequence Synthetic peptide
DVSNRAS

SEQ ID NO: 381 - protein, 7 aa, Artificial Sequence
  Description of Artificial Sequence Synthetic peptide
DASKLQT

SEQ ID NO: 382 - protein, 7 aa, Artificial Sequence
  Description of Artificial Sequence Synthetic peptide
DATNRAT

SEQ ID NO: 383 - protein, 7 aa, Artificial Sequence
  Description of Artificial Sequence Synthetic peptide
EVNQRPS

SEQ ID NO: 384 - protein, 7 aa, Artificial Sequence
  Description of Artificial Sequence Synthetic peptide
GASIRAP

SEQ ID NO: 385 - protein, 7 aa, Artificial Sequence
  Description of Artificial Sequence Synthetic peptide
DVSTRAT

SEQ ID NO: 386 - protein, 7 aa, Artificial Sequence
  Description of Artificial Sequence Synthetic peptide
DTSIRAA

SEQ ID NO: 387 - protein, 7 aa, Artificial Sequence
  Description of Artificial Sequence Synthetic peptide
GASTRAT

SEQ ID NO: 388 - protein, 7 aa, Artificial Sequence
  Description of Artificial Sequence Synthetic peptide
DASKLET

SEQ ID NO: 389 - protein, 7 aa, Artificial Sequence
  Description of Artificial Sequence Synthetic peptide
MATNRAS

SEQ ID NO: 390 - protein, 7 aa, Artificial Sequence
  Description of Artificial Sequence Synthetic peptide
MGSQRAA

SEQ ID NO: 391 - protein, 7 aa, Artificial Sequence
  Description of Artificial Sequence Synthetic peptide
MGSIRAP

SEQ ID NO: 392 - protein, 7 aa, Artificial Sequence
  Description of Artificial Sequence Synthetic peptide
GASSRAA

SEQ ID NO: 393 - protein, 7 aa, Artificial Sequence
  Description of Artificial Sequence Synthetic peptide
RASARES

SEQ ID NO: 394 - protein, 7 aa, Artificial Sequence
  Description of Artificial Sequence Synthetic peptide
RTYNLAS

SEQ ID NO: 395 - protein, 7 aa, Artificial Sequence
  Description of Artificial Sequence Synthetic peptide
EDIERPS

SEQ ID NO: 396 - protein, 7 aa, Artificial Sequence
  Description of Artificial Sequence Synthetic peptide
GASIRAG

SEQ ID NO: 397 - protein, 7 aa, Artificial Sequence
  Description of Artificial Sequence Synthetic peptide
DTTNRPP

SEQ ID NO: 398 - protein, 9 aa, Artificial Sequence
  Description of Artificial Sequence Synthetic peptide
QQYTAYPLT

SEQ ID NO: 399 - protein, 8 aa, Artificial Sequence
  Description of Artificial Sequence Synthetic peptide
QQYFTLPT

SEQ ID NO: 400 - protein, 9 aa, Artificial Sequence
  Description of Artificial Sequence Synthetic peptide
MQGTQAPRT

SEQ ID NO: 401 - protein, 9 aa, Artificial Sequence
  Description of Artificial Sequence Synthetic peptide
VQDLHTPFT

SEQ ID NO: 402 - protein, 9 aa, Artificial Sequence
  Description of Artificial Sequence Synthetic peptide
VQTLQTPST

SEQ ID NO: 403 - protein, 9 aa, Artificial Sequence
  Description of Artificial Sequence Synthetic peptide
LQYGSSPPL

SEQ ID NO: 404 - protein, 9 aa, Artificial Sequence
  Description of Artificial Sequence Synthetic peptide
QQYDTHPLS

SEQ ID NO: 405 - protein, 9 aa, Artificial Sequence
  Description of Artificial Sequence Synthetic peptide
QQYSSYPSL

SEQ ID NO: 406 - protein, 9 aa, Artificial Sequence
  Description of Artificial Sequence Synthetic peptide
VQDLHTPFT

SEQ ID NO: 407 - protein, 9 aa, Artificial Sequence
  Description of Artificial Sequence Synthetic peptide
MQVLRTPIT

SEQ ID NO: 408 - protein, 6 aa, Artificial Sequence
  Description of Artificial Sequence Synthetic peptide
STMAIP

SEQ ID NO: 409 - protein, 10 aa, Artificial Sequence
  Description of Artificial Sequence Synthetic peptide
QQYVSAPPYT

SEQ ID NO: 410 - protein, 9 aa, Artificial Sequence
  Description of Artificial Sequence Synthetic peptide
VQDLHTPFT

SEQ ID NO: 411 - protein, 11 aa, Artificial Sequence
  Description of Artificial Sequence Synthetic peptide
QQYGSSPPVYS

SEQ ID NO: 412 - protein, 9 aa, Artificial Sequence
  Description of Artificial Sequence Synthetic peptide
QQSYSHPPT

SEQ ID NO: 413 - protein, 10 aa, Artificial Sequence
  Description of Artificial Sequence Synthetic peptide
SSYTSSSTDV

SEQ ID NO: 414 - protein, 10 aa, Artificial Sequence
  Description of Artificial Sequence Synthetic peptide
QQYHTWPPWA

SEQ ID NO: 415 - protein, 8 aa, Artificial Sequence
  Description of Artificial Sequence Synthetic peptide
QQYDNPPS

SEQ ID NO: 416 - protein, 9 aa, Artificial Sequence
  Description of Artificial Sequence Synthetic peptide
RQYATFPQT

SEQ ID NO: 417 - protein, 9 aa, Artificial Sequence
  Description of Artificial Sequence Synthetic peptide
LQYTNYPWT

SEQ ID NO: 418 - protein, 11 aa, Artificial Sequence
  Description of Artificial Sequence Synthetic peptide
AAWDASVAGYV

SEQ ID NO: 419 - protein, 9 aa, Artificial Sequence
  Description of Artificial Sequence Synthetic peptide
QQYITWPST

SEQ ID NO: 420 - protein, 10 aa, Artificial Sequence
  Description of Artificial Sequence Synthetic peptide
QQYGSSLPPT

SEQ ID NO: 421 - protein, 7 aa, Artificial Sequence
  Description of Artificial Sequence Synthetic peptide
QQYGNWH

SEQ ID NO: 422 - protein, 9 aa, Artificial Sequence
  Description of Artificial Sequence Synthetic peptide
QQYNDRPRT

SEQ ID NO: 423 - protein, 9 aa, Artificial Sequence
  Description of Artificial Sequence Synthetic peptide
QQAKSFPPT

SEQ ID NO: 424 - protein, 9 aa, Artificial Sequence
  Description of Artificial Sequence Synthetic peptide
VQDLHTLPQ

SEQ ID NO: 425 - protein, 5 aa, Artificial Sequence
  Description of Artificial Sequence Synthetic peptide
QSGVT

SEQ ID NO: 426 - protein, 9 aa, Artificial Sequence
  Description of Artificial Sequence Synthetic peptide
MQGTQAPRT

SEQ ID NO: 427 - protein, 9 aa, Artificial Sequence
  Description of Artificial Sequence Synthetic peptide
MQSTQWPHT

SEQ ID NO: 428 - protein, 9 aa, Artificial Sequence
  Description of Artificial Sequence Synthetic peptide
QQYRSFPWS

SEQ ID NO: 429 - protein, 9 aa, Artificial Sequence
  Description of Artificial Sequence Synthetic peptide
QQRSSYPWT

SEQ ID NO: 430 - protein, 10 aa, Artificial Sequence
  Description of Artificial Sequence Synthetic peptide
QVWDATLDLV

SEQ ID NO: 431 - protein, 9 aa, Artificial Sequence
  Description of Artificial Sequence Synthetic peptide
QQYETWPWT

SEQ ID NO: 432 - protein, 11 aa, Artificial Sequence
  Description of Artificial Sequence Synthetic peptide
QSTERGRSDVI

SEQ ID NO: 433 - protein, 116 aa, Artificial Sequence
  Description of Artificial Sequence Synthetic polypeptide
QVQLQESGPGLVKPSQTLSLTCTVSGLSLSNAKMGVNWIRQPPGKGLEWIGYVSTTGTTDYNPSLKSRVTMSVDTSKNQFSLKVNSVTAADTAVYYCARAGYPDVWGKGTTVTVSS

SEQ ID NO: 434 - protein, 107 aa, Artificial Sequence
  Description of Artificial Sequence Synthetic polypeptide
DIQMTQSPSSVSASVGDRVTITCRASQNIGAALAWYQQKPGKAPKLLIYGASTREYGVPSRFSGSGSGTDFTLTISSLQPEDFANYYCQQYTAYPLTFGGGTKVEIK

SEQ ID NO: 435 - protein, 116 aa, Artificial Sequence
  Description of Artificial Sequence Synthetic polypeptide
QVQLQESGPGLVKPSQTLSLTCTVSGASVSNARTGVNWIRQPPGKGLEWIGYINPSGRIDYNPSLKSRVTMSVDTSKNQFSLKVNSVTAADTAVYYCARAGYPDVWGKGTTVTVSS

SEQ ID NO: 436 - protein, 106 aa, Artificial Sequence
  Description of Artificial Sequence Synthetic polypeptide
EIVMTQSPATLSLSPGERATLSCRASQTVSSHLAWYQQKPGQAPRLLIYDVSHRASGIPARFSGSGSGTDFTLTISSLEPEDFAVYYCQQYFTLPTFGGGTKVEIK

SEQ ID NO: 437 - protein, 125 aa, Artificial Sequence
  Description of Artificial Sequence Synthetic polypeptide
EVQLVESGGGLVQPGGSLRLSCAASGFTASRNYMSWVRQAPGKGLELVASISGSGDYTYYPDSVKGRFTISRDNAKNSLYLQMNSLRAEDTAVYYCARSPRGPRLLGYYYGMDVWGQGTTVTVSS

SEQ ID NO: 438 - protein, 112 aa, Artificial Sequence
  Description of Artificial Sequence Synthetic polypeptide
DIVMTQSPLSLPVTPGEPASISCRSSRSLVHGSGHNYLHWYLQKPGQSPQLLIYGASTRVAGVPDRFSGSGSGTDFTLKISRVEAEDVGVYYCMQGTQAPRTFGGGTKVEIK

SEQ ID NO: 439 - protein, 120 aa, Artificial Sequence
  Description of Artificial Sequence Synthetic polypeptide
EVQLVESGGGLVQPGGSLRLSCAASGFDFGEHWMSWVRQAPGKGLELVASIKGDASDTYYPDSVKGRFTISRDNAKNSLYLQMNSLRAEDTAVYYCVKDLSKQWLGIDYWGQGTLVTVSS

SEQ ID NO: 440 - protein, 112 aa, Artificial Sequence
  Description of Artificial Sequence Synthetic polypeptide
DIVMTQSPLSLPVTPGEPASISCRSSQSLLHSNEKNYLHWYLQKPGQSPQLLIYDASKRASGVPDRFSGSGSGTDFTLKISRVEAEDVGVYYCVQDLHTPFTFGGGTKVEIK

SEQ ID NO: 441 - protein, 123 aa, Artificial Sequence
  Description of Artificial Sequence Synthetic polypeptide
EVQLVESGGGLVQPGGSLRLSCAASGYTFSNYGMSWVRQAPGKGLELVASISKSGSSIYYPDSVKGRFTISRDNAKNSLYLQMNSLRAEDTAVYYCARLPRLAWNYYYGMDVWGQGTTVTVSS

SEQ ID NO: 442 - protein, 112 aa, Artificial Sequence
  Description of Artificial Sequence Synthetic polypeptide
DIVMTQSPLSLPVTPGEPASISCRSSRSLVHGSGDNYLHWYLQKPGQSPQLLIYMASNRAPGVPDRFSGSGSGTDFTLKISRVEAEDVGVYYCVQTLQTPSTFGGGTKVEIK

SEQ ID NO: 443 - protein, 116 aa, Artificial Sequence; identical to SEQ ID NO: 433
  Description of Artificial Sequence Synthetic polypeptide

SEQ ID NO: 444 - protein, 107 aa, Artificial Sequence
  Description of Artificial Sequence Synthetic polypeptide
EIVMTQSPATLSLSPGERATLSCRASQNVYTNLAWYQQKPGQAPRLLIYGASARASGIPARFSGSGSGTDFTLTISSLEPEDFAVYYCLQYGSSPPLFGGGTKVEIK

SEQ ID NO: 445 - protein, 121 aa, Artificial Sequence
  Description of Artificial Sequence Synthetic polypeptide
QVQLQESGPGLVKPSQTLSLTCTVSGVSLSNARLGVNWIRQPPGKGLEWIGYGSVTGSTDYNPSLKSRVTMSVDTSKNQFSLKVNSVTAADTAVYYCTTSYLVDTAMVTRWGQGTLVTVSS

SEQ ID NO: 446 - protein, 107 aa, Artificial Sequence
  Description of Artificial Sequence Synthetic polypeptide
EIVMTQSPATLSLSPGERATLSCRASQSLGTNLAWYQQKPGQAPRLLIYGASSRPSGIPARFSGSGSGTDFTLTISSLEPEDFAVYYCQQYDTHPLSFGGGTKVEIK

SEQ ID NO: 447 - protein, 116 aa, Artificial Sequence
  Description of Artificial Sequence Synthetic polypeptide
QVQLQESGPGLVKPSQTLSLTCTVSGYSLTDARIGVNWIRQPPGKGLEWIGYINPSGRTDYNPSLKSRVTMSVDTSKNQFSLKVNSVTAADTAVYYCARAGYPDVWGKGTTVTVSS

SEQ ID NO: 448 - protein, 107 aa, Artificial Sequence
  Description of Artificial Sequence Synthetic polypeptide
DIQMTQSPSSVSASVGDRVTITCRASQTISRSLAWYQQKPGKAPKLLIYDASSLAGGVPSRFSGSGSGTDFTLTISSLQPEDFANYYCQQYSSYPSLFGGGTKVEIK

SEQ ID NO: 449 - protein, 123 aa, Artificial Sequence; identical to SEQ ID NO: 441
  Description of Artificial Sequence Synthetic polypeptide

SEQ ID NO: 450 - protein, 112 aa, Artificial Sequence; identical to SEQ ID NO: 440
  Description of Artificial Sequence Synthetic polypeptide

SEQ ID NO: 451 - protein, 123 aa, Artificial Sequence
  Description of Artificial Sequence Synthetic polypeptide
EVQLVESGGGLVQPGGSLRLSCAASGFTLSNYPMSWVRQAPGKGLELVASTTSGSSTIYYPDSVKGRFTISRDNAKNSLYLQMNSLRAEDTAVYYCARGPDYYAPLRSYMDVWGKGTTVTVSS

SEQ ID NO: 452 - protein, 112 aa, Artificial Sequence
  Description of Artificial Sequence Synthetic polypeptide
DIVMTQSPLSLPVTPGEPASISCRSSQSLQQSEGRTYLHWYLQKPGQSPQLLIYDASKRASGVPDRFSGSGSGTDFTLKISRVEAEDVGVYYCMQVLRTPITFGGGTKVEIK

SEQ ID NO: 453 - protein, 121 aa, Artificial Sequence; identical to SEQ ID NO: 445
  Description of Artificial Sequence Synthetic polypeptide

SEQ ID NO: 454 - protein, 104 aa, Artificial Sequence
  Description of Artificial Sequence Synthetic polypeptide
EIVMTQSPATLSLSPGERATLSCRASQRIGSDLAWYQQKPGQAPRLLIYSASSRAPGIPARFSGSGSGTDFTLTISSLEPEDFAVYYCSTMAIPFGGGTKVEIK

SEQ ID NO: 455 - protein, 116 aa, Artificial Sequence; identical to SEQ ID NO: 433
  Description of Artificial Sequence Synthetic polypeptide

SEQ ID NO: 456 - protein, 108 aa, Artificial Sequence
  Description of Artificial Sequence Synthetic polypeptide
DIQMTQSPSSVSASVGDRVTITCRASQGISRDLAWYQQKPGKAPKLLIYAASTLVSGVPSRFSGSGSGTDFTLTISSLQPEDFANYYCQQYVSAPPYTFGGGTKVEIK

SEQ ID NO: 457 - protein, 125 aa, Artificial Sequence; identical to SEQ ID NO: 437
  Description of Artificial Sequence Synthetic polypeptide

SEQ ID NO: 458 - protein, 112 aa, Artificial Sequence; identical to SEQ ID NO: 440
  Description of Artificial Sequence Synthetic polypeptide

SEQ ID NO: 459 - protein, 116 aa, Artificial Sequence; identical to SEQ ID NO: 447
  Description of Artificial Sequence Synthetic polypeptide

SEQ ID NO: 460 - protein, 109 aa, Artificial Sequence
  Description of Artificial Sequence Synthetic polypeptide
EIVMTQSPATLSLSPGERATLSCRASQSIGTKLAWYQQKPGQAPRLLIYDASSRVPGIPARFSGSGSGTDFTLTISSLEPEDFAVYYCQQYGSSPPVYSFGGGTKVEIK

SEQ ID NO: 461 - protein, 121 aa, Artificial Sequence
  Description of Artificial Sequence Synthetic polypeptide
QVTLRESGPALVKPTQTLTLTCTVSGFSLSTSGATVNWIRQPPGKALEWIGYITSSGRTVYNSALKSRLTISKDTSKNQVVLTMTNMDPVDTATYYCTTTKIVGATLRDYWGQGTLVTVSS

SEQ ID NO: 462 - protein, 113 aa, Artificial Sequence
  Description of Artificial Sequence Synthetic polypeptide
DIVMTQSPDSLAVSLGERATINCKSSQSVLDTSTNRNYLAWYQQKPGQPPKLLIYGASIRAGGVPDRFSGSGSGTDFTLTISSLQAEDVAVYYCQQSYSHPPTFGGGTKVEIK

SEQ ID NO: 463 - protein, 126 aa, Artificial Sequence
  Description of Artificial Sequence Synthetic polypeptide
EVQLVQSGAEVKKPGASVKVSCKASGYTYTGYRISWVRQAPGQGLEWMGWIDPKSGRTNYAQKLQGRGTMTTDPSTSTAYMELRSLRSDDTAVYYCAREVKQWLVPGRGVYWFDPWGQGTLVTVSS

SEQ ID NO: 464 - protein, 107 aa, Artificial Sequence
  Description of Artificial Sequence Synthetic polypeptide
ESALTQPASVSGSPGQSITISCTGTNIGLRSVSWYQQHPGKAPKLMIYDTNIRSSGVSNRFSGSKSGNTASLTISGLQAEDEADYYCSSYTSSSTDVFGGGTKLTVL

SEQ ID NO: 465 - protein, 116 aa, Artificial Sequence
  Description of Artificial Sequence Synthetic polypeptide
QVQLQESGPGLVKPSQTLSLTCTVSGVSLSDARMGVNWIRQPPGKGLEWIGYVSTTGTTDYNPSLKSRVTMSVDTSKNQFSLKVNSVTAADTAVYYCARAGYPDVWGKGTLVTVSS

SEQ ID NO: 466 - protein, 108 aa, Artificial Sequence
  Description of Artificial Sequence Synthetic polypeptide
EIVMTQSPATLSLSPGERATLSCRASESVRSNLAWYQQKPGQAPRLLIYGASNRASGIPARFSGSGSGTDFTLTISSLEPEDFAVYYCQQYHTWPPWAFGGGTKVEIK

SEQ ID NO: 467 - protein, 121 aa, Artificial Sequence; identical to SEQ ID NO: 461
  Description of Artificial Sequence Synthetic polypeptide

SEQ ID NO: 468 - protein, 106 aa, Artificial Sequence
  Description of Artificial Sequence Synthetic polypeptide
DIQMTQSPSSVSASVGDRVTITCRASQGISNDLAWYQQKPGKAPKLLIYDVSNRASGVPSRFSGSGSGTDFTLTISSLQPEDFANYYCQQYDNPPSFGGGTKVEIK

SEQ ID NO: 469 - protein, 116 aa, Artificial Sequence
  Description of Artificial Sequence Synthetic polypeptide
QVTLRESGPALVKPTQTLTLTCTVSGFTLSNPRMGVNWIRQPPGKGLEWIGYISTSGRTDYNPSLKSRVTMSVDTSKNQFSLKVNSVTAADTAVYYCARAGYPDVWGKGTTVTVSS

SEQ ID NO: 470 - protein, 107 aa, Artificial Sequence
  Description of Artificial Sequence Synthetic polypeptide
DIQMTQSPSSVSASVGDRVTITCRASQGIGIDLAWYQQKPGKAPKLLIYDASKLQTGVPSRFSGSGSGTDFTLTISSLQPEDFANYYCRQYATFPQTFGGGTKVEIK

SEQ ID NO: 471 - protein, 116 aa, Artificial Sequence
  Description of Artificial Sequence Synthetic polypeptide
QVQLQESGPGLVKPSQTLSLTCTVSGFPLSTSRMGVNWIRQPPGKGLEWIGYINPSGATDYNPSLKSRVTMSVDTSKNQFSLKVNSVTAADTAVYYCARAGYPDVWGKGTTVTVSS

SEQ ID NO: 472 - protein, 107 aa, Artificial Sequence
  Description of Artificial Sequence Synthetic polypeptide
EIVMTQSPATLSLSPGERATLSCRASQSVSTHLAWYQQKPGQAPRLLIYDATNRATGIPARFSGSGSGTDFTLTISSLEPEDFAVYYCLQYTNYPWTFGGGTKVEIK

SEQ ID NO: 473 - protein, 127 aa, Artificial Sequence
  Description of Artificial Sequence Synthetic polypeptide
EVQLVQSGAEVKKPGASVKVSCKASGYTFPSHYISWVRQAPGQGLEWMGGMNPITGHTIYAQKLQGRGTMTTDPSTSTAYMELRSLRSDDTAVYYCARDMRYDILPGRSYYYGMDVWGQGTTVTVSS

SEQ ID NO: 474 - protein, 108 aa, Artificial Sequence
  Description of Artificial Sequence Synthetic polypeptide
ESALTQPASVSGSPGQSITISCTGTDIRGMSVSWYQQHPGKAPKLMIYEVNQRPSGVSNRFSGSKSGNTASLTISGLQAEDEADYYCAAWDASVAGYVFGGGTKLTVL

SEQ ID NO: 475 - protein, 116 aa, Artificial Sequence; identical to SEQ ID NO: 471
  Description of Artificial Sequence Synthetic polypeptide

SEQ ID NO: 476 - protein, 107 aa, Artificial Sequence
  Description of Artificial Sequence Synthetic polypeptide
EIVMTQSPATLSLSPGERATLSCRASQNIGHSLAWYQQKPGQAPRLLIYGASIRAPGIPARFSGSGSGTDFTLTISSLEPEDFAVYYCQQYITWPSTFGGGTKVEIK

SEQ ID NO: 477 - protein, 116 aa, Artificial Sequence; identical to SEQ ID NO: 471
  Description of Artificial Sequence Synthetic polypeptide

SEQ ID NO: 478 - protein, 108 aa, Artificial Sequence
  Description of Artificial Sequence Synthetic polypeptide
EIVMTQSPATLSLSPGERATLSCRASRSVSSDLAWYQQKPGQAPRLLIYDVSTRATGIPARFSGSGSGTDFTLTISSLEPEDFAVYYCQQYGSSLPPTFGGGTKVEIK

SEQ ID NO: 479 - protein, 116 aa, Artificial Sequence; identical to SEQ ID NO: 471
  Description of Artificial Sequence Synthetic polypeptide

SEQ ID NO: 480 - protein, 105 aa, Artificial Sequence
  Description of Artificial Sequence Synthetic polypeptide
EIVMTQSPATLSLSPGERATLSCRASQSVRTSLAWYQQKPGQAPRLLIYDTSIRAAGIPARFSGSGSGTDFTLTISSLEPEDFAVYYCQQYGNWHFGGGTKVEIK

SEQ ID NO: 481 - protein, 116 aa, Artificial Sequence
  Description of Artificial Sequence Synthetic polypeptide
QVQLQESGPGLVKPSQTLSLTCTVSGVSLSNVRMGVNWIRQPPGKGLEWIGYVNPSGSTDYNPSLKSRVTMSVDTSKNQFSLKVNSVTAADTAVYYCARAGYPDVWGKGTLVTVSS

SEQ ID NO: 482 - protein, 108 aa, Artificial Sequence
  Description of Artificial Sequence Synthetic polypeptide
EIVMTQSPATLSLSPGERATLSCRASQSISSSQLAWYQQKPGQAPRLLIYGASTRATGIPARFSGSGSGTDFTLTISSLEPEDFAVYYCQQYNDRPRTFGGGTKVEIK

SEQ ID NO: 483 - protein, 118 aa, Artificial Sequence
  Description of Artificial Sequence Synthetic polypeptide
QVQLVQSGAEVKKPGASVKVSCKVSGYTFTSSAISWVRQAPGKGLEWMGGIYPGSGHTIYAQKFQGRVTMTEDTSTDTAYMELSSLKSEDTAVYYCAREGDIFVMDVWGLGTAVTVSS

SEQ ID NO: 484 - protein, 107 aa, Artificial Sequence
  Description of Artificial Sequence Synthetic polypeptide
DIQMTQSPSSVSASVGDRVTITCRASMDIRNDLAWYQQKPGKAPKLLIYDASKLETGVPSRFSGSGSGTDFTLTISSLQPEDFANYYCQQAKSFPPTFGGGTKVEIK

SEQ ID NO: 485 - protein, 124 aa, Artificial Sequence
  Description of Artificial Sequence Synthetic polypeptide
EVQLVESGGGLVQPGGSLRLSCAASGFTSSNHWMSWVRQAPGKGLELVASIGPKGLGTYYPDSVKGRFTISRDNAKNSLYLQMNSLRAEDTAVYYCASIRDYGDKKGGSWFDPWGQGTLVTVSS

SEQ ID NO: 486 - protein, 112 aa, Artificial Sequence
  Description of Artificial Sequence Synthetic polypeptide
DIVMTQSPLSLPVTPGEPASISCRSSQSLLHSSGYNFLHWYLQKPGQSPQLLIYMATNRASGVPDRFSGSGSGTDFTLKISRVEAEDVGVYYCVQDLHTLPQFGGGTKVEIK

SEQ ID NO: 487 - protein, 116 aa, Artificial Sequence
  Description of Artificial Sequence Synthetic polypeptide
EVQLVESGGGLVQPGGSLRLSCAASGFTFSDHHMSWVRQAPGKGLELVASISPSGDFTYYPDSVKGRFTISRDNAKNSLYLQMNSLRAEDTAVYYCARDVSPVYYWGQGALVTVSS

SEQ ID NO: 488 - protein, 100 aa, Artificial Sequence
  Description of Artificial Sequence Synthetic polypeptide
DIVMTQSPLSLPVTPGEPASISCRSSQSLLHWYLQKPGQSPQLLIYMGSQRAAGVPDRFSGSGSGTDFTLKISRVEAEDVGVYYCQSGVTFGGGTKVEIK

SEQ ID NO: 489 - protein, 121 aa, Artificial Sequence
  Description of Artificial Sequence Synthetic polypeptide
EVQLVESGGGLVQPGGSLRLSCAASGFTSSGYWMSWFRQAPGKGLELVASIGTAGVTYYPDSVKGRFTISRDNAKNSLYLQMNSLRAEDTAVYYCARGPHGPQQRLRFDPWGQGTLVTVSS

SEQ ID NO: 490 - protein, 112 aa, Artificial Sequence
  Description of Artificial Sequence Synthetic polypeptide
DIVMTQSPLSLPVTPGEPASISCRSSQSLLSSYGYHNLHWYLQKPGQSPQLLIYMGSIRAPGVPDRFSGSGSGTDFTLKISRVEAEDVGVYYCMQGTQAPRTFGGGTKVEIK

SEQ ID NO: 491 - protein, 125 aa, Artificial Sequence
  Description of Artificial Sequence Synthetic polypeptide
EVQLVESGGGLVQPGGSLRLSCAASGFTSSNHWMSWVRQAPGKGLELVASIGPKGLGTYYPDSVKGRFTISRDNAKNSLYLQMNSLRAEDTAVYYCARDVRGWRALYYYYGMDVWGQGTTVTVSS

SEQ ID NO: 492 - protein, 112 aa, Artificial Sequence
  Description of Artificial Sequence Synthetic polypeptide
DIVMTQSPLSLPVTPGEPASISCRSSQSLLHVTGYNYLHWYLQKPGQSPQLLIYGASSRAAGVPDRFSGSGSGTDFTLKISRVEAEDVGVYYCMQSTQWPHTFGGGTKVEIK

SEQ ID NO: 493 - protein, 118 aa, Artificial Sequence
  Description of Artificial Sequence Synthetic polypeptide
QVQLVQSGAEVKKPGASVKVSCKVSGYPSPATISWVRQAPGKGLEWMGWMNPKSGYTNYAQKFQGRVTMTEDTSTDTAYMELSSLKSEDTAVYYCATLHGSGLAFDIWGQGTMVTVSS

SEQ ID NO: 494 - protein, 107 aa, Artificial Sequence
  Description of Artificial Sequence Synthetic polypeptide
DIQMTQSPSSVSASVGDRVTITCRASQDIKSDLAWYQQKPGKAPKLLIYRASARESGVPSRFSGSGSGTDFTLTISSLQPEDFANYYCQQYRSFPWSFGGGTKVEIK

SEQ ID NO: 495 - protein, 120 aa, Artificial Sequence
  Description of Artificial Sequence Synthetic polypeptide
QVQLQQPGTELVKPGASVKLSCKASGYTFTSYWMHWVKQRPGQGLEWIGNINPSNGGTNYNEKFKSKATLTVDKSSSTAYMQLSSLTSEDSAVYYCSITTVVAYWYFDVWGTGTTVTVSS

SEQ ID NO: 496 - protein, 106 aa, Artificial Sequence
  Description of Artificial Sequence Synthetic polypeptide
ENVLTQSPAIMSASPGEKVTISCSASSSVSYMYWYQQKPGSSPKPWIFRTYNLASGVPARFSGSGSGTSYSLTISNMEAEDAATYYCQQRSSYPWTFGGGTKLEIK

SEQ ID NO: 497 - protein, 123 aa, Artificial Sequence
  Description of Artificial Sequence Synthetic polypeptide
EVQLVQSGAEVKKPGASVKVSCKASGDTSSHSHIHWVRQAPGQGLEWMGWINPRSGATNYAQKLQGRGTMTTDPSTSTAYMELRSLRSDDTAVYYCARDRGGRTNGVGAFDIWGQGTMVTVSS

SEQ ID NO: 498 - protein, 110 aa, Artificial Sequence
  Description of Artificial Sequence Synthetic polypeptide
ESALTQPASVSGSPGQSITISCTGTSGDIGRYNYVSWYQQHPGKAPKLMIYEDIERPSGVSNRFSGSKSGNTASLTISGLQAEDEADYYCQVWDATLDLVFGGGTKLTVL

SEQ ID NO: 499 - protein, 122 aa, Artificial Sequence
  Description of Artificial Sequence Synthetic polypeptide
QVTLRESGPALVKPTQTLTLTCTVSGFSLSDTRWGVNWIRQPPGKALEWIGYINDRGTTDYNSALKSRLTISKDTSKNQVVLTMTNMDPVDTATYYCAHTLHTREFVGMDVWGQGTTVTVSS

SEQ ID NO: 500 - protein, 113 aa, Artificial Sequence
  Description of Artificial Sequence Synthetic polypeptide
DIVMTQSPDSLAVSLGERATINCKSSQSVLDTSTNRNYLAWYQQKPGQPPKLLIYGASIRAGGVPDRFSGSGSGTDFTLTISSLQAEDVAVYYCQQYETWPWTFGGGTKVEIK

SEQ ID NO: 501 - protein, 127 aa, Artificial Sequence; identical to SEQ ID NO: 473
  Description of Artificial Sequence Synthetic polypeptide

SEQ ID NO: 502 - protein, 110 aa, Artificial Sequence
  Description of Artificial Sequence Synthetic polypeptide
ESALTQPASVSGSPGQSITISCTGTTGSIASNYVSWYQQHPGKAPKLMIYDTTNRPPGVSNRFSGSKSGNTASLTISGLQAEDEADYYCQSTERGRSDVIFGGGTKLTVL

(19) United States
(12) Patent Application Publication
(10) Pub. No.: US 2025/0059267 A1
(43) Pub. Date: Feb. 20, 2025

(54) COMBINATION THERAPY FOR TREATMENT OF TUMORS COMPRISING CANCER-ASSOCIATED FIBROBLASTS

(71) Applicant: PHENOMIC AI, Toronto (CA)

(72) Inventors: Sam COOPER, Toronto (CA); Allison NIXON, Toronto (CA); Max LONDON, Toronto (CA); Elizabeth KOCH, Toronto (CA); Arif JETHA, Toronto (CA); Christopher HARVEY, Boston, MA (US); Michael BRISKIN, Boston, MA (US)

(21) Appl. No.: 18/568,262

(22) PCT Filed: May 13, 2022
(86) PCT No.: PCT/US2022/029323

§ 371 Date: Dec. 7, 2023

Related U.S. Application Data
(60) Provisional application No. 63/189,046, filed on May 14, 2021.

(51) Int. Cl. C07K 16/22; A61K 45/06; A61P 35/00

(52) U.S. Cl. CPC C07K 16/22; A61K 45/06; A61P 35/00

(57) ABSTRACT

The disclosure provides compositions and methods comprising at least one TGF-0 inhibitor and at least one Activin inhibitor for use in methods of treating CAF-enriched cancers and/or fibrotic disease.

### CROSS-REFERENCE TO RELATED APPLICATIONS

This application claims the benefit of priority to U.S. Provisional Application No. 63/189,046 filed on May 14, 2021.

### FIELD OF INVENTION

The present disclosure relates generally to methods for treating solid tumors enriched in cancer fibroblasts. In some embodiments, the methods comprises administering a TGF-β inhibitor, and an activin inhibitor.

### BACKGROUND OF THE INVENTION

Cancer associated fibroblasts (CAFs) are the dominant cell type in the tumor stroma, a cellular compartment that is consistently implicated in resistance to current standard of care. For example, key studies have shown that CAF infiltrates correlate with immunotherapy (IO) response/non-response, confer resistance to chemotherapy, and define more aggressive cancer sub-types with poor prognosis (see, e.g., Tongyan Liu et al. in J. Hematol. Oncol. 12.1 (2019): 86; Linda Ziani et al. in Front. Immunol. 9 (2018): 414; Lucy V. Ireland in Front Cell Dev Biol 6 (2018): 131; Raghu Kalluri in Front Cell Dev Biol 6 (2018): 131; Erik Sahai et al. in Nat. Rev. Cancer 20.3 (2020); Alexandre Calon in Nat. Genet. 47.4 (2015): 320-29). Together these results suggest significant therapeutic value in blocking either the transformation of normal tissue fibroblasts into CAFs, or the pro-tumor effects of CAFs on the tumor microenvironment.

Despite this correlation with pro-tumorigenic activity, CAFs are also known to play tumor suppressive roles in certain cancers, for example, limiting cancer growth rate in certain breast cancers, and suppressing cancer cell stemness in oral cancer (see e.g., Alice Santi et al. in Proteomics 18.5-6 (2018): e1700167., Pamela Maris et al. in PLoS Med. 12.9 (2015): e1001871.; Ankit Kumar Patel et al. in Oncogenesis 7.10 (2018): 78.). That CAFs exert both pro- and anti-tumor effects on the microenvironment is likely a key reason why efforts to broadly target CAFs in the clinic have failed to demonstrate therapeutic efficacy, despite promising pre-clinical data (see e.g., Sanjeev Mariathasan et al. in Nature 554.7693 (2018); Daniele V. F. Tauriello et al. in Nature 554.7693 (2018): 538-43). For example, the Fibroblast Associated Protein (FAP) targeting agent sibrotuzumab failed to demonstrate clinical benefit in metastatic colorectal cancer, despite depletion of FAP positive fibroblasts in pre-clinical models having a significant impact on tumor growth rates (see R-D Hofheinz et al. in Oncology Research and Treatment, (2003); Andrew M. Scott et al. in Clin. Cancer Res. 9.5 (2003): 1639-47; C. Feig et al. in Proceedings of the National Academy of Sciences (2013)). Thus, to effectively drug the tumor stroma in cancer, therapies are needed that selectively target the pro-tumor effects of CAFs (Sahai et al. 2020).

TGF-β is now considered a driver of immune suppression in the tumor microenvironment. This has been demonstrated bioinformatically (Calon et al. 2015) and by association of expression levels of TGF-β in cell types such as stromal fibroblasts in immunotherapy resistant tumors (see Rik Derynck et al. Nat. Rev. Clin. Oncol. 18.1 (2021): 9-34). Furthermore there is preclinical evidence that combination TGF-β inhibition can be additive with immune checkpoint therapy in preclinical models that mimic immunosuppressed clinical subsets (Mariathasan et al. 2018). In spite of these data, the combination of anti TGF-β and anti-PD1 has failed to result in complete tumor elimination as has been seen with combinations in more immunogenic tumors suggesting the need for other combination therapies in patients not responding to current standard of care (Mariathasan et al. 2018; Tauriello et al. 2018).

Accordingly, improved strategies are needed for targeting cancer-associated fibroblasts in CAF-enriched cancers and to thwart aggressive cancer sub-types with poor prognosis. Accordingly, provided herein are methods, cells, compositions, and systems to provide for these and other needs.

### SUMMARY OF INVENTION

The present disclosure provides compositions and methods combining at least one TGF-β inhibitor with at least one Activin inhibitor for the treatment of CAF-enriched cancers, e.g. breast, pancreatic, colorectal, head and neck, and lung cancers and the like, as well as other fibrotic diseases. The present disclosure is based in part on the unexpected observation that inhibiting a combination of targets in the same TGF-β superfamily signaling pathway produces better inhibition of tumor growth and/or tumor volume than that observed when the only one member of the signaling pathway is targeted. As surprisingly demonstrated herein for the first time, the subject methods and compositions can inhibit the transformation of normal tissue fibroblasts into cancer-associated fibroblasts. In some embodiments, the combination therapy further comprises an immune checkpoint inhibitor, to increase the response rate and overall survival in response to cancer treatment in a subject. Remarkably, then, the present disclosure provides additional and improved therapeutic options for treating cancers producing solid tumors that have developed resistance to conventional therapeutic approaches.

Accordingly, in one aspect the present invention provides a method for treating a solid tumor comprising cancer-associated fibroblasts (CAF) in a subject in need thereof, the method comprising: administering simultaneously or sequentially to the subject a therapeutically effective dose of at least one TGF-β inhibitor and the at least one Activin inhibitor. In some embodiments, the tumor is from a cancer selected from the group comprising or consisting of e.g., breast, pancreatic, colorectal, head and neck, and lung cancers. In some embodiments, the TGF-β inhibitor and the Activin inhibitor are each independently selected from a peptidic inhibitor and an antibody inhibitor. In some embodiments, the TGF-β inhibitor is an antibody inhibitor selected from the group consisting of anti-TGF-β antibody clone 1D11, Fresolimumab, galunisertib, and LY3022859; and the Activin inhibitor is an antibody inhibitor selected from the group consisting of Garetosmab, and AMGA1, or the peptidic inhibitor, Follistatin Fc. In some embodiments, the TGF-β inhibitor targets TGF-β 1. In some embodiments, the TGF-β inhibitor further targets TGF-β3. In some embodiments, the Activin inhibitor inhibits Activin A and may also include inhibition of Activin B.

In some embodiments, the Activin inhibitor is an anti-activin antibody disclosed in co-pending International Application No. PCT/US2021/065079, the disclosure of which is expressly incorporated by reference herein. In some embodiments, the anti-activin antibody comprises a heavy chain variable region/light chain variable region (HCVR/LCVR) sequence pair selected from the group consisting of SEQ ID NOs: 433/434, 435/436, 437/438, 439/440, 441/442, 443/444, 445/446, 447/448, 449/450, 451/452, 453/454, 455/456, 457/458, 459/460, 461/462, 463/464, 465/466, 467/468, 469/470, 471/472, 473/474, 475/476, 477/478, 479/480, 481/482, 483/484, 485/486, 487/488, 489/490, 491/492, 493/494, 495/496, 497/498, 499/500, and 501/502. In some embodiments, the anti-activin antibody comprises the six complementarity determining regions (CDRs) of a HCVR/LCVR sequence pair selected from the group consisting of SEQ ID NOs: 433/434, 435/436, 437/438, 439/440, 441/442, 443/444, 445/446, 447/448, 449/450, 451/452, 453/454, 455/456, 457/458, 459/460, 461/462, 463/464, 465/466, 467/468, 469/470, 471/472, 473/474, 475/476, 477/478, 479/480, 481/482, 483/484, 485/486, 487/488, 489/490, 491/492, 493/494, 495/496, 497/498, 499/500, and 501/502.

In some embodiments, the Activin inhibitor inhibits both Activin A and Activin B, where the Activin inhibitor is an anti-activin antibody comprising a HCVR/LCVR sequence pair selected from the group consisting of SEQ ID NOs: 471/472, 473/474, 475/476, 477/478, 479/480, 481/482, 495/496, 497/498, and 501/502; or is an anti-activin antibody comprising the six CDRs of a HVCR/LCVR sequence pair selected from the group consisting of SEQ ID NOs: 471/472, 473/474, 475/476, 477/478, 479/480, 481/482, 495/496, 497/498, and 501/502.

In some embodiments, the administering is systemic administration. In some embodiments, the therapeutically effective dose of the TGF-β inhibitor and the Activin inhibitor given in combination is lower than the therapeutically effective dose of either inhibitor given individually. In some embodiments, the therapeutically effective dose of the Activin inhibitor is higher than the therapeutically effective dose of the TGF-β inhibitor. In some embodiments, the composition further comprises an immune checkpoint inhibitor. In some embodiments, the immune checkpoint inhibitor is a PD-1 checkpoint inhibitor.

Another aspect of the disclosure provides a method for treating a subject having metastasis of a primary tumor comprising cancer-associated fibroblasts, the method comprising simultaneously or sequentially administering to the subject a therapeutically effective dose of at least one a TGF-β inhibitor and at least one an Activin inhibitor. In some embodiments, the primary tumor is selected from the group comprising or consisting of e.g., breast, pancreatic, colorectal, head and neck, and lung cancers. In some embodiments, the metastasis resides in the liver, bone, brain, or other organs. In some embodiments, the TGF-β inhibitor and the Activin inhibitor are each independently selected from a peptidic inhibitor and an antibody inhibitor. In some embodiments, the TGF-β inhibitor is an antibody inhibitor selected from the group consisting of anti-TGF-β antibody clone 1D11, Fresolimumab, galunisertib, and LY3022859; and the Activin inhibitor is an antibody inhibitor selected from the group consisting of Garetosmab, AMGA1, or the peptidic inhibitor, Follistatin Fc. In some embodiments, the TGF-β inhibitor targets TGF-β1. In some embodiments, the TGF-β inhibitor further targets TGF-β3. In some embodiments, the Activin inhibitor inhibits Activin A and/or Activin B. In some embodiments, the Activin inhibitor is an anti-activin antibody disclosed in co-pending International Application No. PCT/US2021/065079.

In some embodiments, the anti-activin antibody comprises a heavy chain variable region/light chain variable region (HCVR/LCVR) sequence pair selected from the group consisting of SEQ ID NOs: 433/434, 435/436, 437/438, 439/440, 441/442, 443/444, 445/446, 447/448, 449/450, 451/452, 453/454, 455/456, 457/458, 459/460, 461/462, 463/464, 465/466, 467/468, 469/470, 471/472, 473/474, 475/476, 477/478, 479/480, 481/482, 483/484, 485/486, 487/488, 489/490, 491/492, 493/494, 495/496, 497/498, 499/500, and 501/502. In some embodiments, the anti-activin antibody comprises the six complementarity determining regions (CDRs) of a HCVR/LCVR sequence pair selected from the group consisting of SEQ ID NOs: 433/434, 435/436, 437/438, 439/440, 441/442, 443/444, 445/446, 447/448, 449/450, 451/452, 453/454, 455/456, 457/458, 459/460, 461/462, 463/464, 465/466, 467/468, 469/470, 471/472, 473/474, 475/476, 477/478, 479/480, 481/482, 483/484, 485/486, 487/488, 489/490, 491/492, 493/494, 495/496, 497/498, 499/500, and 501/502.

In some embodiments, the Activin inhibitor inhibits both Activin A and Activin B, where the Activin inhibitor is an anti-activin antibody comprising a HCVR/LCVR sequence pair selected from the group consisting of SEQ ID NOs: 471/472, 473/474, 475/476, 477/478, 479/480, 481/482, 495/496, 497/498, and 501/502; or is an anti-activin antibody comprising the six CDRs of a HVCR/LCVR sequence pair selected from the group consisting of SEQ ID NOs: 471/472, 473/474, 475/476, 477/478, 479/480, 481/482, 495/496, 497/498, and 501/502.

In some embodiments, the administering is systemic administration. In some embodiments, the therapeutically effective dose(s) of the TGF-β inhibitor and the Activin inhibitor given in combination is lower than the therapeutically effective doses of either inhibitor given individually. In some embodiments, the therapeutically effective dose of the Activin inhibitor is higher than the therapeutically effective dose of the TGF-β inhibitor. In some embodiments, the composition further comprises an immune checkpoint inhibitor. In some embodiments, the immune checkpoint inhibitor is a PD-1 checkpoint inhibitor.

Another aspect of the disclosure provides a method of inhibiting the growth of a solid tumor enriched in cancer fibroblasts in a patient in need thereof, the method comprising: administering simultaneously or sequentially to said patient at least one a TGF-β inhibitor and at least one an Activin inhibitor. In some embodiments, the solid tumor is from e.g., breast, pancreatic, colorectal, head and neck, and lung cancers. In some embodiments, the TGF-β inhibitor and the Activin inhibitor are each independently selected from a peptidic inhibitor and an antibody inhibitor. In some embodiments, the TGF-β inhibitor is an antibody inhibitor selected from the group consisting of anti-TGF-β antibody clone 1D11, Fresolimumab, galunisertib, and LY3022859; and the Activin inhibitor is an antibody inhibitor selected from the group consisting of Garetosmab, and AMGA1, or the peptidic inhibitor, Follistatin Fc. In some embodiments, the TGF-β inhibitor targets TGF-β 1. In some embodiments, the TGF-β inhibitor further targets TGF-β3. In some embodiments, the Activin inhibitor inhibits Activin A and/or Activin B.

In some embodiments, the Activin inhibitor is an anti-activin antibody disclosed in co-pending International Application No. PCT/US2021/065079. In some embodiments, the anti-activin antibody comprises a heavy chain variable region/light chain variable region (HCVR/LCVR) sequence pair selected from the group consisting of SEQ ID NOs: 433/434, 435/436, 437/438, 439/440, 441/442, 443/444, 445/446, 447/448, 449/450, 451/452, 453/454, 455/456, 457/458, 459/460, 461/462, 463/464, 465/466, 467/468, 469/470, 471/472, 473/474, 475/476, 477/478, 479/480, 481/482, 483/484, 485/486, 487/488, 489/490, 491/492, 493/494, 495/496, 497/498, 499/500, and 501/502. In some embodiments, the anti-activin antibody comprises the six complementarity determining regions (CDRs) of a HCVR/LCVR sequence pair selected from the group consisting of SEQ ID NOs: 433/434, 435/436, 437/438, 439/440, 441/442, 443/444, 445/446, 447/448, 449/450, 451/452, 453/454, 455/456, 457/458, 459/460, 461/462, 463/464, 465/466, 467/468, 469/470, 471/472, 473/474, 475/476, 477/478, 479/480, 481/482, 483/484, 485/486, 487/488, 489/490, 491/492, 493/494, 495/496, 497/498, 499/500, and 501/502.

In some embodiments, the Activin inhibitor inhibits both Activin A and Activin B, where the Activin inhibitor is an anti-activin antibody comprising a HCVR/LCVR sequence pair selected from the group consisting of SEQ ID NOs: 471/472, 473/474, 475/476, 477/478, 479/480, 481/482, 495/496, 497/498, and 501/502; or is an anti-activin antibody comprising the six CDRs of a HVCR/LCVR sequence pair selected from the group consisting of SEQ ID NOs: 471/472, 473/474, 475/476, 477/478, 479/480, 481/482, 495/496, 497/498, and 501/502.

In some embodiments, the administering is systemic administration. In some embodiments, the administering is by intra-tumoral injection. In some embodiments, the therapeutically effective dose of the TGF-β inhibitor and the Activin inhibitor given in combination is lower than the therapeutically effective dose of either inhibitor given individually. In some embodiments, the therapeutically effective dose of the Activin inhibitor is higher than the therapeutically effective dose of the TGF-β inhibitor. In some embodiments, the composition further comprises an immune modulating agent or cell-therapy. In some embodiments, the composition further comprises an immune modulator is an immune checkpoint inhibitor. In some embodiments, the immune checkpoint inhibitor is a PD-1 checkpoint inhibitor.

Another aspect of the disclosure provides a method for treating a fibrotic disease in a subject in need thereof, the method comprising: administering to the subject a therapeutically effective dose of at least one TGF-β inhibitor and at least one Activin inhibitor. In some embodiments, the fibrotic disease is selected from the group comprising or consisting of Pulmonary Fibrosis (PF), Idiopathic Pulmonary Fibrosis (IPF), Liver Cirrhosis, Non-alcoholic steric hepatitis (NASH), Pulmonary Arterial Hypertension (PAH), Renal Fibrosis, Chronic Kidney Disease (CKD), Chronic Dermal Fibrosis, Scleroderma, Hypertrophic Scar, Cardiac Fibrosis, Hypertrophic Cardiomyopathy, and in some aspects Alzheimer's Disease and Inflammatory Bowel Disease.

In some embodiments, the TGF-β inhibitor and the Activin inhibitor are each independently selected from a peptidic inhibitor and an antibody inhibitor. In some embodiments, the TGF-β inhibitor is an antibody inhibitor selected from the group consisting of anti-TGF-β antibody clone 1D11, Fresolimumab, galunisertib, and LY3022859; and the Activin inhibitor is an antibody inhibitor selected from the group consisting of Garetosmab, and AMGA1, or the peptidic inhibitor, Follistatin Fc. In some embodiments, the TGF-β inhibitor targets TGF-β1. In some embodiments, the TGF-β inhibitor further targets TGF-β3. In some embodiments, the Activin inhibitor inhibits Activin A and/or Activin B.

In some embodiments, the Activin inhibitor is an anti-activin antibody disclosed in co-pending International Application No. PCT/US2021/065079. In some embodiments, the anti-activin antibody comprises a heavy chain variable region/light chain variable region (HCVR/LCVR) sequence pair selected from the group consisting of SEQ ID NOs: 433/434, 435/436, 437/438, 439/440, 441/442, 443/444, 445/446, 447/448, 449/450, 451/452, 453/454, 455/456, 457/458, 459/460, 461/462, 463/464, 465/466, 467/468, 469/470, 471/472, 473/474, 475/476, 477/478, 479/480, 481/482, 483/484, 485/486, 487/488, 489/490, 491/492, 493/494, 495/496, 497/498, 499/500, and 501/502. In some embodiments, the anti-activin antibody comprises the six complementarity determining regions (CDRs) of a HCVR/LCVR sequence pair selected from the group consisting of SEQ ID NOs: 433/434, 435/436, 437/438, 439/440, 441/442, 443/444, 445/446, 447/448, 449/450, 451/452, 453/454, 455/456, 457/458, 459/460, 461/462, 463/464, 465/466, 467/468, 469/470, 471/472, 473/474, 475/476, 477/478, 479/480, 481/482, 483/484, 485/486, 487/488, 489/490, 491/492, 493/494, 495/496, 497/498, 499/500, and 501/502.

In some embodiments, the Activin inhibitor inhibits both Activin A and Activin B, where the Activin inhibitor is an anti-activin antibody comprising a HCVR/LCVR sequence pair selected from the group consisting of SEQ ID NOs: 471/472, 473/474, 475/476, 477/478, 479/480, 481/482, 495/496, 497/498, and 501/502; or is an anti-activin antibody comprising the six CDRs of a HVCR/LCVR sequence pair selected from the group consisting of SEQ ID NOs: 471/472, 473/474, 475/476, 477/478, 479/480, 481/482, 495/496, 497/498, and 501/502.

In some embodiments, the administering is systemic administration. In some embodiments, the therapeutically effective dose(s) of the TGF-β inhibitor and the Activin A/B Activin inhibitor given in combination is lower than the therapeutically effective doses of either inhibitor given individually. In some embodiments, the therapeutically effective dose of the Activin A/B Activin inhibitor is higher than the therapeutically effective dose of the TGF-β inhibitor. In some embodiments, the composition further comprises an immune checkpoint inhibitor. In some embodiments, the immune checkpoint inhibitor is a PD-1 checkpoint inhibitor.

Another aspect of the disclosure provides a pharmaceutical composition comprising at least one TGF-β inhibitor and at least one Activin inhibitor in a pharmaceutically acceptable carrier. In some embodiments, the TGF-β inhibitor and the Activin inhibitor are each independently selected from a peptidic inhibitor and an antibody inhibitor. In some embodiments, the TGF-β inhibitor is an antibody inhibitor selected from the group consisting of anti-TGF-β antibody clone 1D11, Fresolimumab, galunisertib, and LY3022859; and the Activin inhibitor is an antibody inhibitor selected from the group consisting of Garetosmab, and AMGA1, or the peptidic inhibitor Follistatin Fc. In some embodiments, the pharmaceutical composition further comprises an immune checkpoint inhibitor. In some embodiments, the immune checkpoint inhibitor is a PD-1 checkpoint inhibitor.

In some embodiments, the TGF-β inhibitor is an antibody inhibitor selected from the group consisting of anti-TGF-β antibody clone 1D11, Fresolimumab, galunisertib, and LY3022859; and the Activin inhibitor is an anti-activin antibody disclosed in co-pending International Application No. PCT/US2021/065079. In some embodiments, the anti-activin antibody comprises a heavy chain variable region/light chain variable region (HCVR/LCVR) sequence pair selected from the group consisting of SEQ ID NOs: 433/434, 435/436, 437/438, 439/440, 441/442, 443/444, 445/446, 447/448, 449/450, 451/452, 453/454, 455/456, 457/458, 459/460, 461/462, 463/464, 465/466, 467/468, 469/470, 471/472, 473/474, 475/476, 477/478, 479/480, 481/482, 483/484, 485/486, 487/488, 489/490, 491/492, 493/494, 495/496, 497/498, 499/500, and 501/502. In some embodiments, the anti-activin antibody comprises the six complementarity determining regions (CDRs) of a HCVR/LCVR sequence pair selected from the group consisting of SEQ ID NOs: 433/434, 435/436, 437/438, 439/440, 441/442, 443/444, 445/446, 447/448, 449/450, 451/452, 453/454, 455/456, 457/458, 459/460, 461/462, 463/464, 465/466, 467/468, 469/470, 471/472, 473/474, 475/476, 477/478, 479/480, 481/482, 483/484, 485/486, 487/488, 489/490, 491/492, 493/494, 495/496, 497/498, 499/500, and 501/502.

In some embodiments, the Activin inhibitor inhibits both Activin A and Activin B, where the Activin inhibitor is an anti-activin antibody comprising a HCVR/LCVR sequence pair selected from the group consisting of SEQ ID NOs: 471/472, 473/474, 475/476, 477/478, 479/480, 481/482, 495/496, 497/498, and 501/502; or is an anti-activin antibody comprising the six CDRs of a HVCR/LCVR sequence pair selected from the group consisting of SEQ ID NOs: 471/472, 473/474, 475/476, 477/478, 479/480, 481/482, 495/496, 497/498, and 501/502. In some embodiments, the pharmaceutical composition further comprises an immune checkpoint inhibitor. In some embodiments, the immune checkpoint inhibitor is a PD-1 checkpoint inhibitor.

In another aspect the present invention provides a use of at least one TGF-β inhibitor and at least one an Activin inhibitor for the manufacture of a medicament for use in the treatment of fibrotic disease, or for use in the treatment of a solid tumor enriched in cancer fibroblasts. In some embodiments, the TGF-β inhibitor and the Activin inhibitor are each independently selected from a peptidic inhibitor and an antibody inhibitor. In some embodiments, the TGF-β inhibitor is an antibody inhibitor selected from the group consisting of anti-TGF-β antibody clone 1D11, Fresolimumab, galunisertib, and LY3022859; and the Activin inhibitor is an antibody inhibitor selected from the group consisting of Garetosmab, and AMGA1, or the peptidic inhibitor, Follistatin Fc. In some embodiments, the TGF-β inhibitor targets TGF-β 1. In some embodiments, the TGF-β inhibitor further targets TGF-β3. In some embodiments, the Activin inhibitor inhibits Activin A and/or Activin B. In some embodiments, the Activin inhibitor is an anti-activin antibody disclosed in co-pending International Application No. PCT/US2021/065079. For example, in some embodiments, the anti-activin antibody comprises a heavy chain variable region/light chain variable region (HCVR/LCVR) sequence pair selected from the group consisting of SEQ ID NOs: 433/434, 435/436, 437/438, 439/440, 441/442, 443/444, 445/446, 447/448, 449/450, 451/452, 453/454, 455/456, 457/458, 459/460, 461/462, 463/464, 465/466, 467/468, 469/470, 471/472, 473/474, 475/476, 477/478, 479/480, 481/482, 483/484, 485/486, 487/488, 489/490, 491/492, 493/494, 495/496, 497/498, 499/500, and 501/502. In some embodiments, the anti-activin antibody comprises the six complementarity determining regions (CDRs) of a HCVR/LCVR sequence pair selected from the group consisting of SEQ ID NOs: 433/434, 435/436, 437/438, 439/440, 441/442, 443/444, 445/446, 447/448, 449/450, 451/452, 453/454, 455/456, 457/458, 459/460, 461/462, 463/464, 465/466, 467/468, 469/470, 471/472, 473/474, 475/476, 477/478, 479/480, 481/482, 483/484, 485/486, 487/488, 489/490, 491/492, 493/494, 495/496, 497/498, 499/500, and 501/502.

In some embodiments, the Activin inhibitor inhibits Activin A and also inhibits Activin B, where the Activin inhibitor is an anti-activin antibody comprising a HCVR/LCVR sequence pair selected from the group consisting of SEQ ID NOs: 471/472, 473/474, 475/476, 477/478, 479/480, 481/482, 495/496, 497/498, and 501/502; or is an anti-activin antibody comprising the six CDRs of a HVCR/LCVR sequence pair selected from the group consisting of SEQ ID NOs: 471/472, 473/474, 475/476, 477/478, 479/480, 481/482, 495/496, 497/498, and 501/502.

In some embodiments, the administering is systemic administration. In some embodiments, the therapeutically effective dose of the TGF-β inhibitor and the Activin inhibitor given in combination is lower than the therapeutically effective dose of either inhibitor given individually. In some embodiments, the therapeutically effective dose of the Activin A/B Activin inhibitor is higher than the therapeutically effective dose of the TGF-β inhibitor. In some embodiments, the composition further comprises an immune checkpoint inhibitor. In some embodiments, the immune checkpoint inhibitor is a PD-1 checkpoint inhibitor.

Other features, objects, and advantages will be apparent to the skilled practitioner from the disclosure that follows.

### INCORPORATION BY REFERENCE

All publications, patents, and patent applications mentioned in this specification are herein incorporated by reference to the same extent as if each individual publication, patent, or patent application was specifically and individually indicated to be incorporated by reference BRIEF DESCRIPTION OF THE DRAWINGS

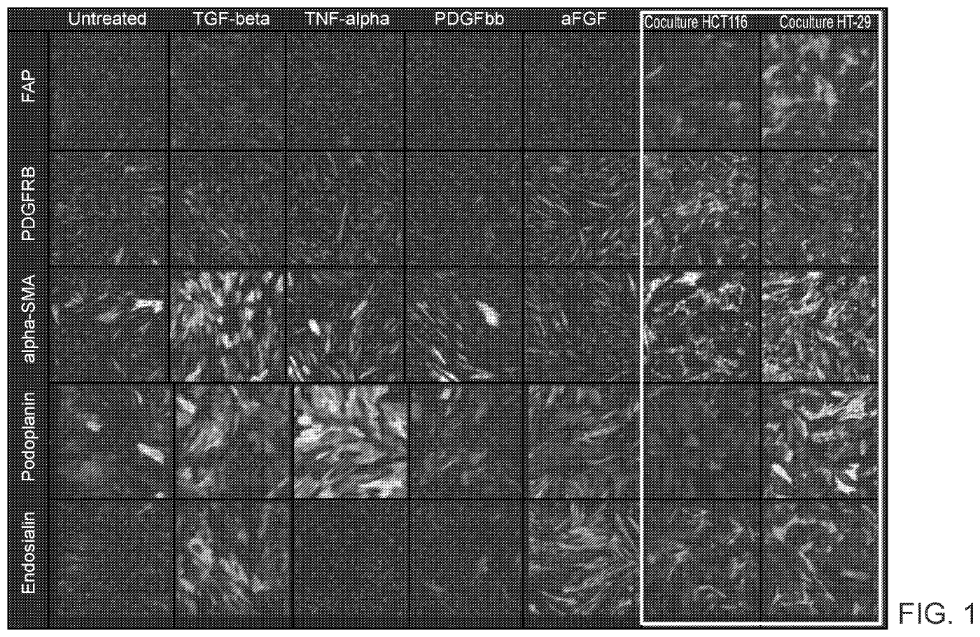

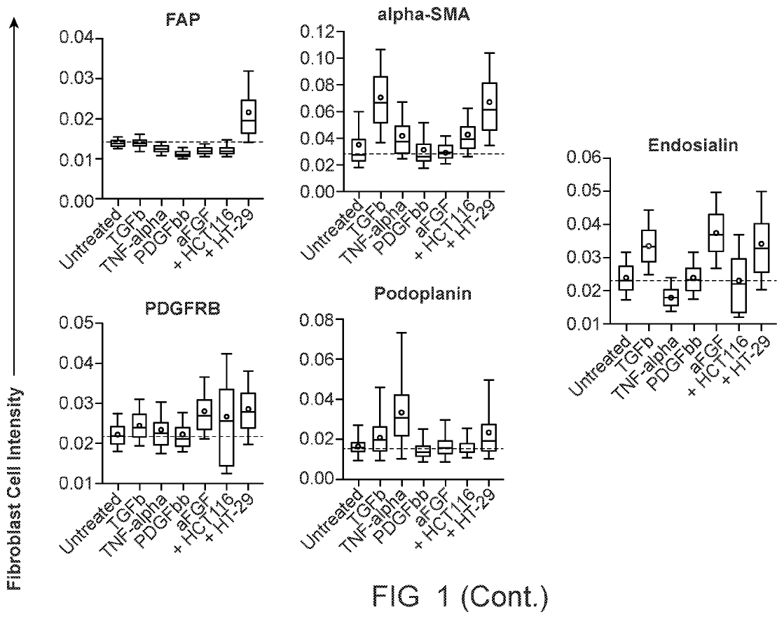

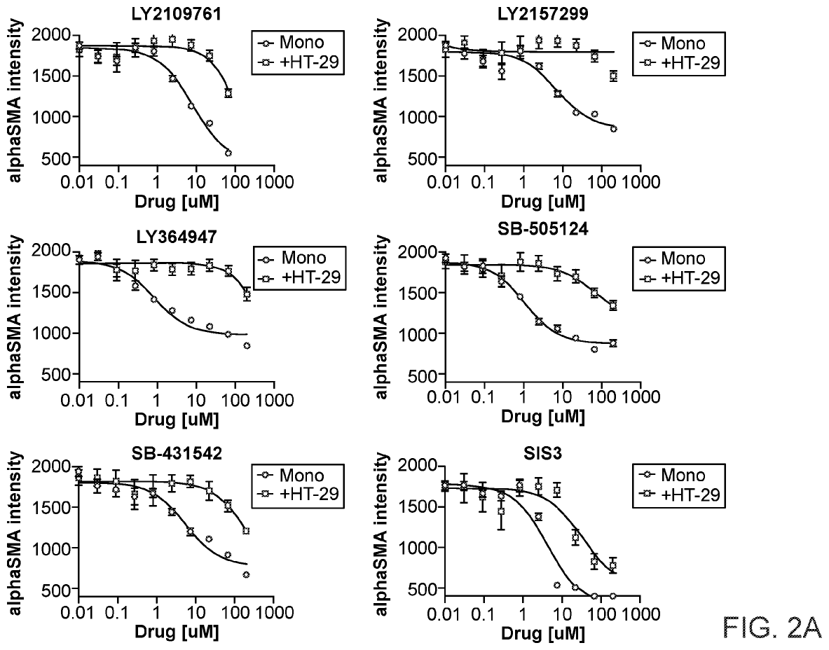

FIG. 1 Fibroblast-cancer co-cultures lead to a greater increase in CAF activation markers than treatment with a single cytokine alone, indicating a cocktail of factors are needed to induce more disease relevant CAFs. CCD-18Co normal colon fibroblasts were either cultured in minimal media (Untreated), with the addition of a cytokine (TGF-β 1.2 ng/mL, TNF-α 1 ng/mL, PDGFbb 4 ng/mL, or aFGF 2 ng/mL), or co-cultured in minimal media with HCT116 or HT-29 colon cancer cells. A panel of 5 CAF markers were then used to stain the cultures and the expression levels of expression of the markers calculated from individual segmented fibroblasts. Analysis of expression levels shows that co-culture with specific lines e.g. HT116, is needed to up-regulate expression of proteins seen in human tumors, such as FAP.

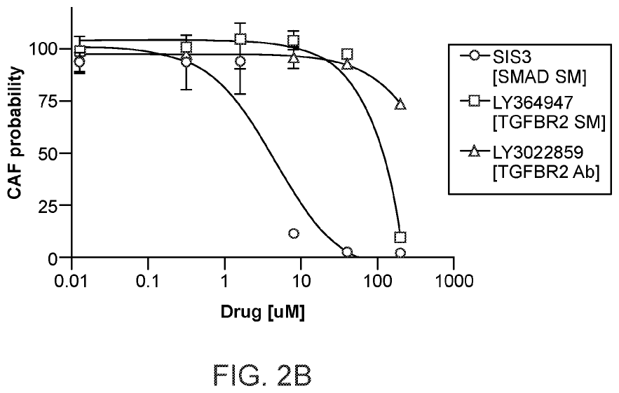

FIG. 2 TGF-β inhibitors perform poorly in co-culture compared to mono-culture. (A) Normal BJ fibroblasts were cultured in either minimal media or alongside HT-29 CRC cancer cells. 6-different TGF-β inhibitors were added alongside TGF-β [1 ng/ml]ligand. Significantly higher IC50 values were observed in all co-culture vs. mono-cultures. By ranking compounds by their IC50 of TGFBR inhibition in cell-free assays, we see more selective compounds have a greater shift in their IC50 value between mono-culture and co-culture. (B) This is most apparent comparing the effects of three TGF-β inhibitors with varying selectivity. SIS3 which inhibits SMAD3 (downstream of both TGF-β receptors and Activin receptors) is able to block CAF activation in coculture. LY364947 is a small molecule inhibitor (SMi) of TGFBR2 also blocks CAF activation at high doses, which are known to also inhibit other TGF-β superfamily members. Finally, LY3022859 is a monoclonal antibody that is highly specific TGFBR2 shows very little effect in coculture. Together this demonstrates that co-cultures are resistant to TGF-β inhibition.

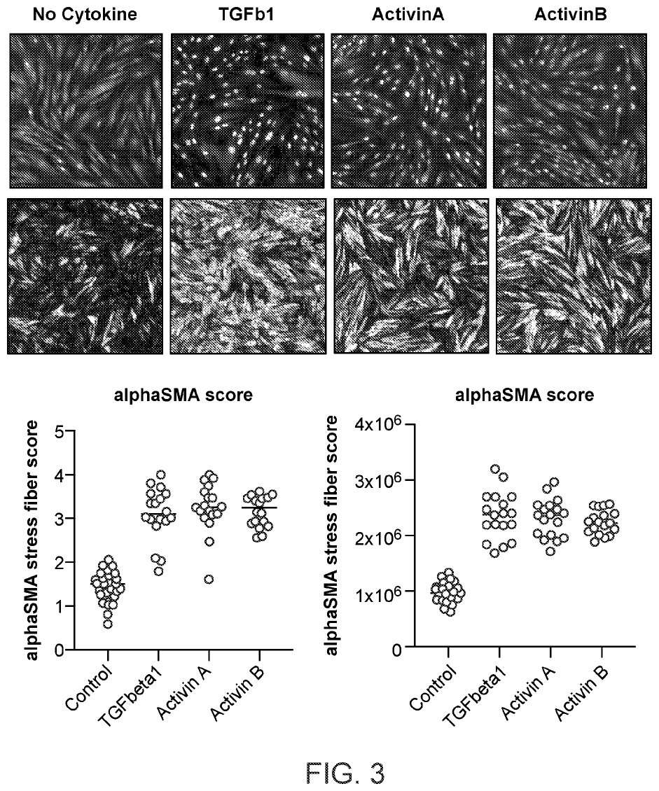

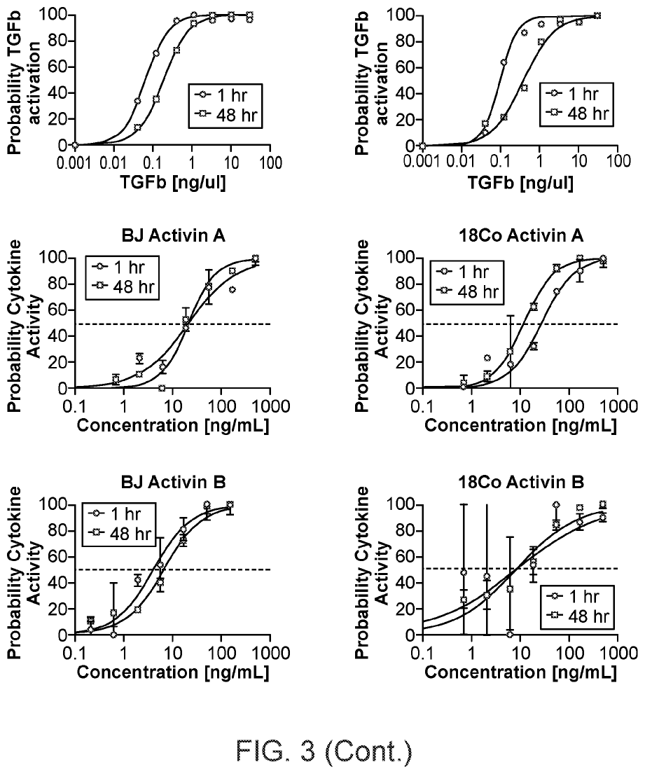

FIG. 3 Activin and TGF-β both induce a CAF phenotype in fibroblast cell-lines. A) Staining of BJ-fibroblasts with SMAD2/3, phospho-CREB, ASMA, Endosialin and Phalloidin shows that TGF-β 1, Activin A and Activin B all induce similar phenotypic changes in fibroblasts. Namely SMAD2/3 translocates to the nucleus, phospho-CREB and ASMA are up-regulated while Endosialin (CD248) is slightly down-regulated. B) Quantification of ASMA levels and plate coverage further reinforce these changes downstream of addition of both Activin and TGF-β. C) TGF-β and Activin A were added over a range of doses to BJ or CCD18-Co colon fibroblasts grown as mono-cultures in minimal media (N=2). Cells were stained for Actin, Endosialin/CD248, DAPI/Nuclei, and αSMA. Machine Learning (Multiple Instance Learning) models were trained on the highest and lowest doses to generate dose-response curves. IC50 values for TGF-β were lower for 1 hr timepoints vs. 48 hr timepoints. Conversely, Activin A IC50 values were similar at both 1 and 48 hr timepoints in BJ fibroblasts, and lower at the 48 hr timepoint in CCD18 colon fibroblasts. Overall this figure shows that Activin A and TGF-β have similar effects on fibroblastic cells and can both induce CAF like phenotypes in vitro.

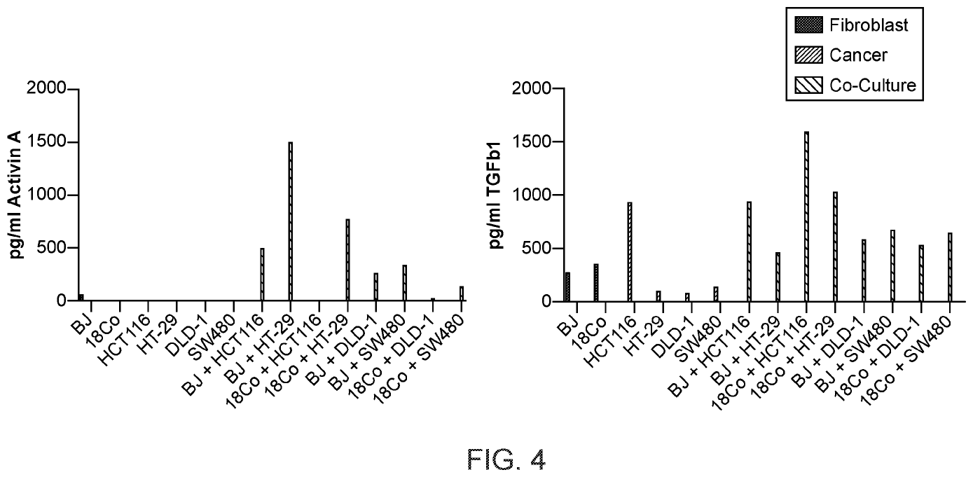

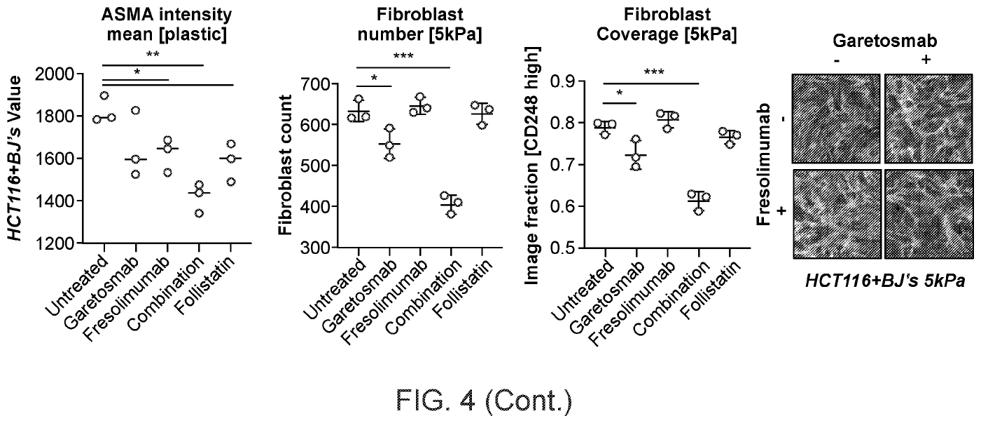

FIG. 4 Activin is uniquely up-regulated in co-cultures and a combination Activin and TGF-β inhibition blocks CAF induction in co-culture models in various culture conditions. A) We sought to profile Activin and TGF-β in co-culture to establish whether both cytokines are expressed, and determine if there would be value in blocking both cytokines in co-culture. Supernatant was harvested from both mono-cultures and co-cultures of BJ and CCD18-Co fiborblasts, alongside HCT116 or HT29 colorectal cancer cell lines. ELISA's were run to quantify concentrations of Activin A and TGF-β—overall this demonstrated that Activin A is uniquely up-regulated in co-culture, whilst TGF-β is expressed in fibroblast and cancer cell mono-cultures but up-regulated in co-culture; B) HCT116 cancer cells were seeded in co-culture with BJ normal fibroblasts for 24 hours on substrates of various stiffnesses, starved and treated with Garetosmab (100 nM), Fresolimumab (100 nM), Follistatin (2 ug/mL) or the Garetosmab and Fresolimumab combination for 48 hours. Co-cultures were fixed and stained with αSMA, CD248 as well as Hoescht and Phalloidin. Fibroblasts were segmented by CellProfiler using a combination of nuclear and Phalloidin features, then αSMA and CD248 features were calculated. Treatment with a combination of Activin and TGF-β inhibitors led to a more significant drop in αSMA intensity, fibroblast count and fibroblast area than either agent alone.

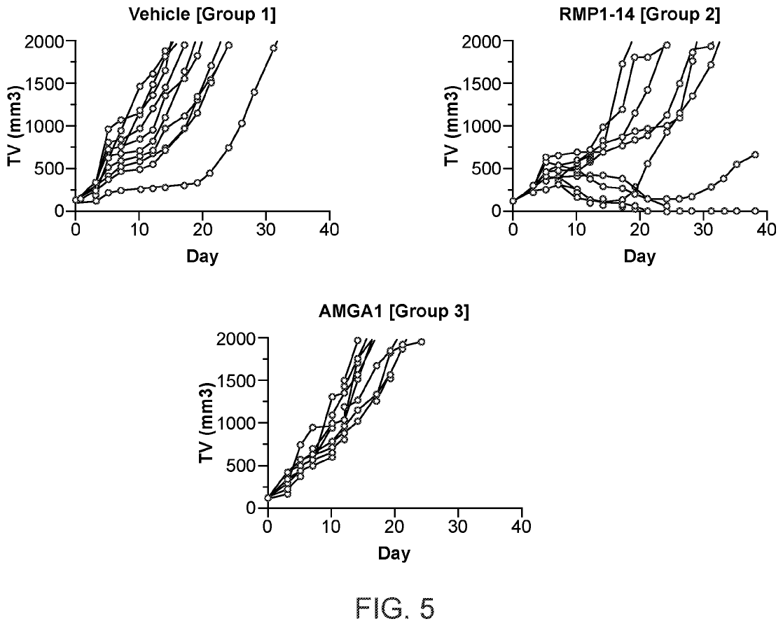

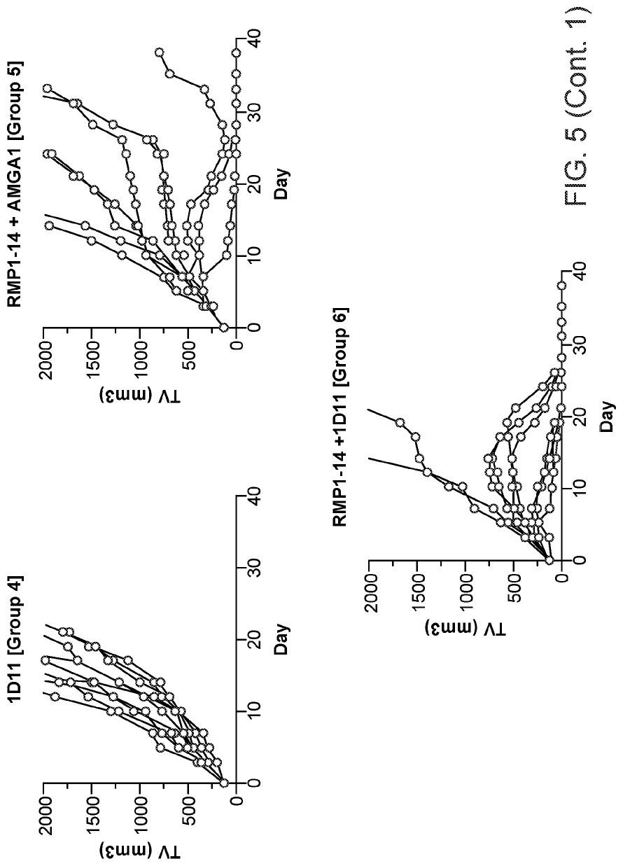

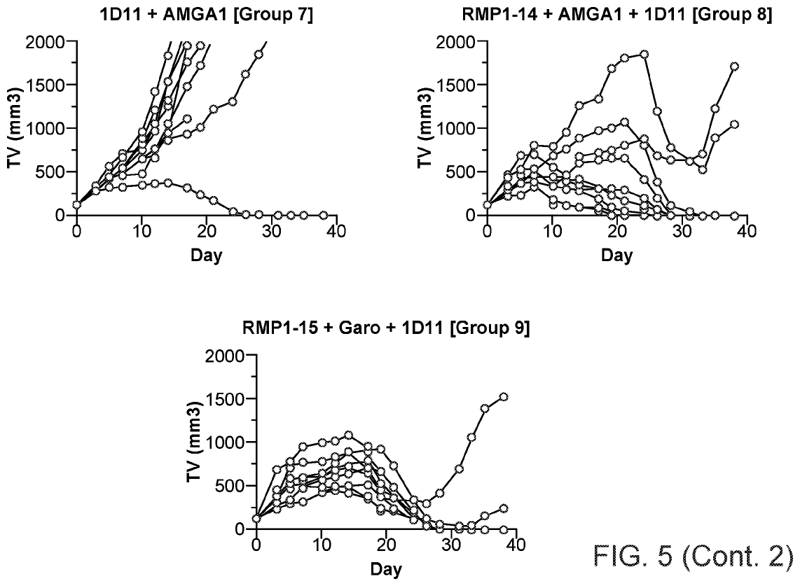

FIG. 5 Triple inhibition of TGF-β, Activin, and PD1 induced tumor regression in a higher percentage of mice than treatment with any mono-therapy or combination thereof. Mice bearing subcutaneous EMT6 tumors mice were treated with vehicle, monoclonal antibodies targeting TGF-β (1D111), PD1 (RMP1-14), the anti-activin A antagonistic antibodies garetosmab and AMGA1, described in U.S. Pat. Nos. 9,718,881 and 8,309,082 respectively, and combinations thereof. Dosing was initiated on day 0 (tumor volume=128 mm3), with 3 doses administered (as per example 2), up until 4 weeks or 28 days. PD1 blockade demonstrated remission at day 28 in 4/10 tumors (tumor volume<2000 mm3). TGF-β blockade in combination with PD1 inhibition demonstrated a response in 8/10 tumors (P=0.168 vs. PD1 alone; cumulative binomial probability). PD1 in combination with TGF-β and Activin A inhibition showed response in 10/10 tumors at day 28 following treatment with both garetosmab and AMGA1, (P<0.01 in both cases). Thus, this figure demonstrates the benefit of blocking both TGF-β and Activin in combination with immune-checkpoint inhibition.

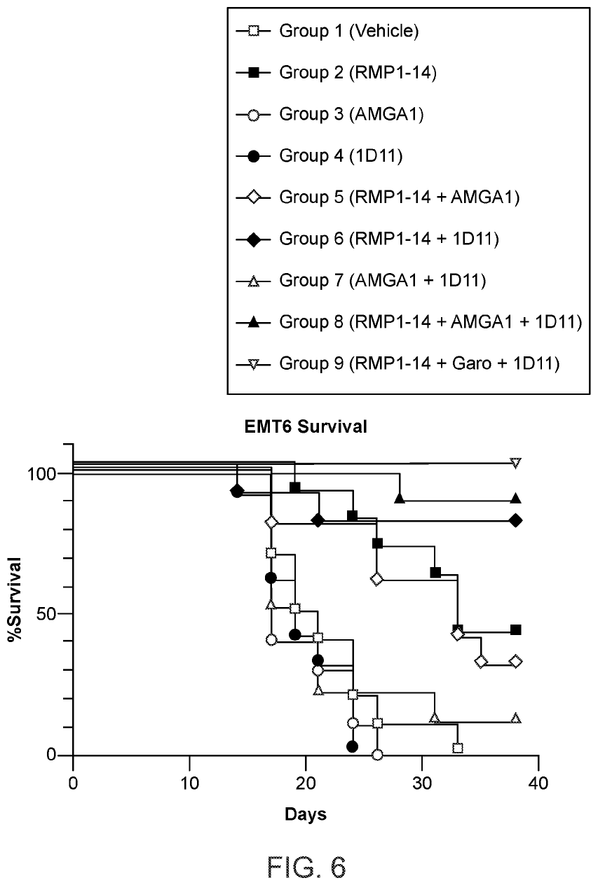

FIG. 6 Triple combination of PD-1 checkpoint inhibitor, a TGF-β inhibitor, and an activin inhibitor increase survival in EMT6 tumor-bearing mice. Survival curves were determined for mice bearing EMT6 tumors. The show that TGF-β and Activin A inhibition alone, as well as the combination of TGF-β and Actvin blockade result in no meaningful improvement in survival vs. control vehicle treatment. Treatment with PD-1, or PD1 and Activin results in a highly significant improvement in survival vs. vehicle (P<0.01 in both cases). Treatment with PD-1+RMP1-14 results in a very highly significant improvement in survival vs. control (P<0.001), although insignificant improvement vs. RMP1-14 alone (P=0.12). This is contrast to the combination of Activin and TGF-β blockade that results in both a very highly significant increase in survival vs. control (P<0.001) and vs. RMP1-14 alone (P<0.05 AMGA1, P<0.01 garetosmab). As such, this experiment shows that a combination of TGF-β and Activin blockade is needed to see a significant improvement in survival vs. monotherapy with the immune-checkpoint inhibitor RMP-14.

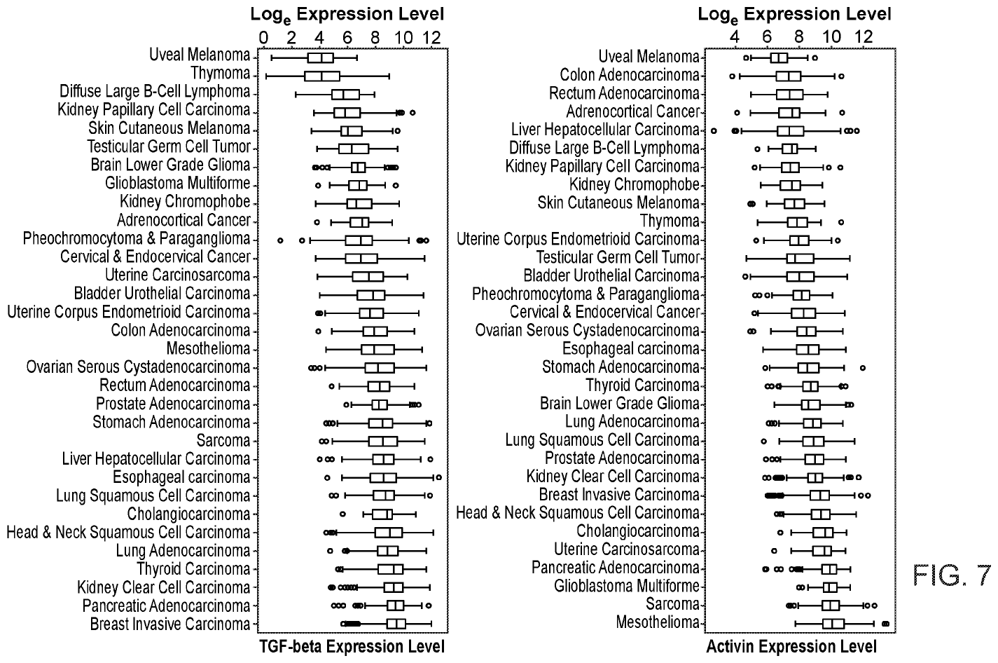

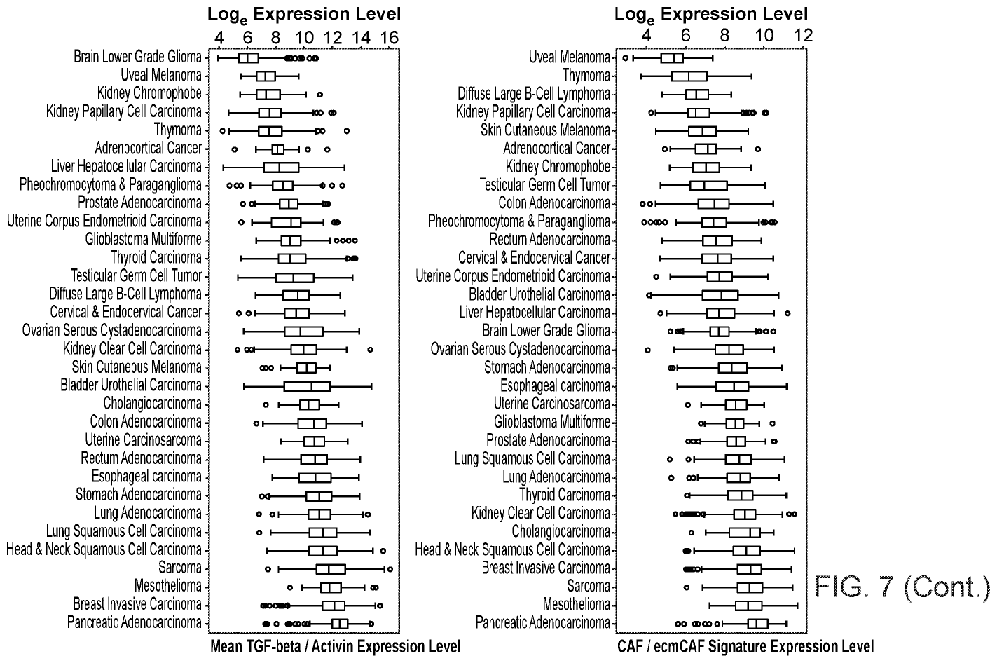

FIG. 7 Common cancers are ranked by mRNA expression levels of Activin, TGF-β, Activin and TGF-β, as well as the signature of an CAF sub-type linked to immune-suppression. mRNA expression level scores were calculated for Activin (mean of: INHBA, INHBB), TGF-β (mean of TGFB1, TGFB2, TGFB3), Activin and TGF-β (mean of: INHBA, INHBB, TGFB1, TGFB2, TGFB3), and an ecmCAF phenotype linked to immune non-response and disease progression (mean of COL1A1, COL1A2, LRRC15, POSTN, FAP, SERPINE1) for all primary tumor samples belonging to common cancer types in The Cancer Genome Atlas (‘TCGA’). Tumors were ranked by mean expression level, and box-plots were derived showing the natural logarithm of the mean, upper and lower quartiles, as well as whiskers giving the upper and lower quartile+1.5 times the interquartile range. These highlight that cancers traditionally considered to be stromal rich rise to the top of the rankings. Taking an averaged TGF-β and Activin score results in a ranking that is more significantly correlated to the ecmCAF ranking than the ranking of either cytokine alone (Spearman's Rank Correlations of 0.67 for Activin vs. CAF signature, P<0.001; 0.58 for TGF-β vs. CAF signature, P<0.001; 0.7 for TGF-β averaged with Activin vs. CAF signature P<0.001).

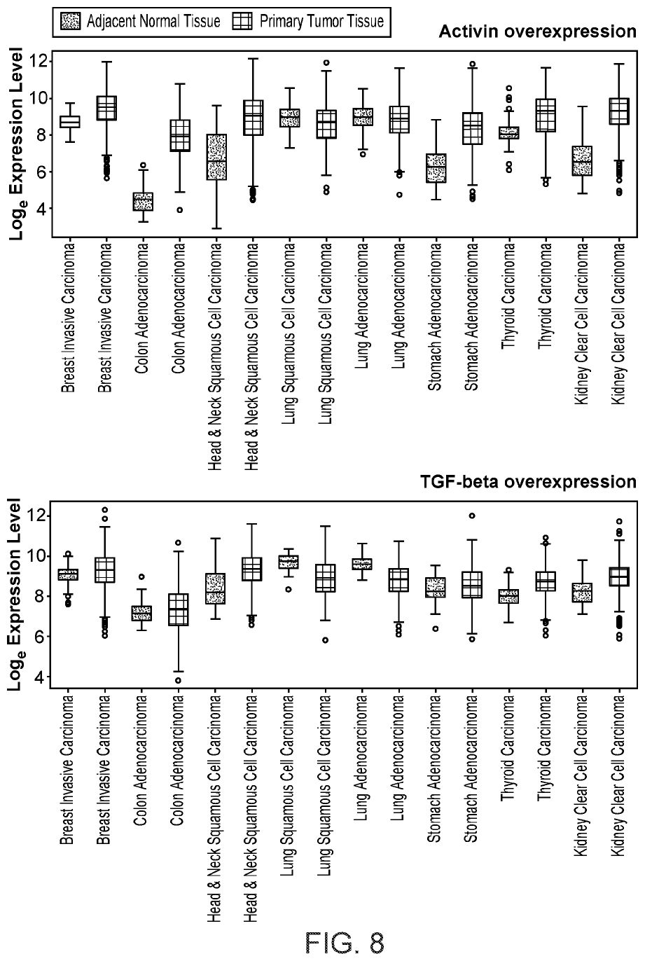

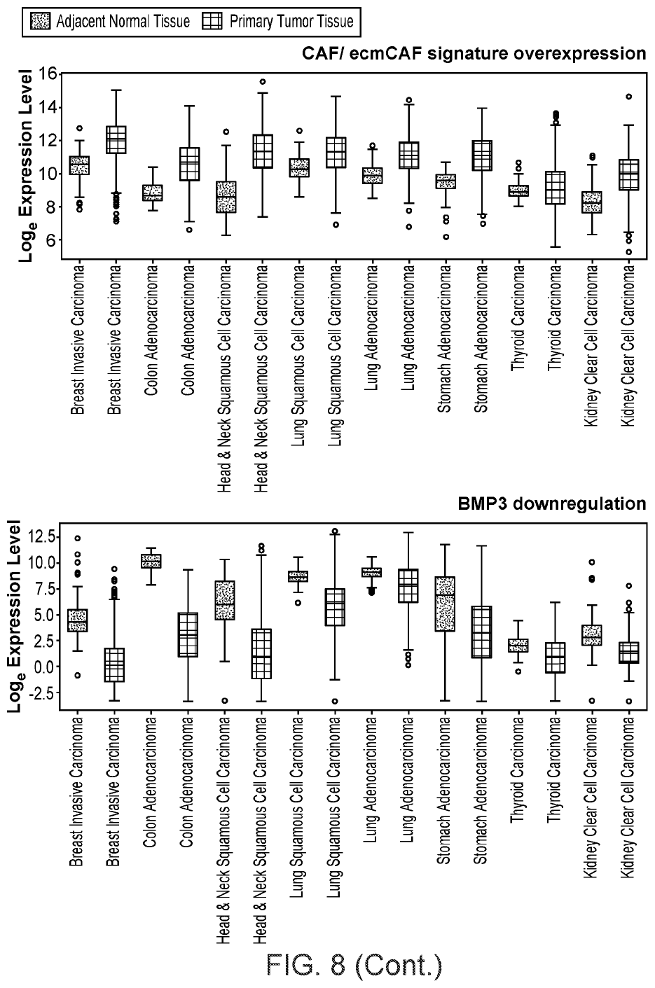

FIG. 8 Dysregulation of mRNA expression levels of Activin, TGF-β, ecmCAF genes and BMP3 in cancerous tissue vs. normal tissue. mRNA expression level scores were calculated for Actvin, TGF-β, the ecmCAF, and BMP3 (BMP3 mRNA only), for both TCGA tissue samples labelled as adjacent, and as primary tumor. Box plots comparing the log mRNA expression level score in adjacent ‘normal’ tissue vs. cancer tissue are shown for stromal rich cancers with greater than 20 adjacent samples. This for example excluded Pancreatic cancer where there were only 4 adjacent samples. Statistical significance measures were excluded from the plot since population differences were very highly significant (P<0.001) for all compared populations. Of note, TGF-β and Activin are up-regulated in all of these stromal rich cancers with the exception of the two lung cancers analysed where these cytokines are downregulated. The ecmCAF signature was up-regulated in every cancer however the fold change was small in Thyroid cancer. Finally BMP3 (considered an inhibitor of Activin signaling) was downregulated in all tested cancers. Overall this analysis highlights that both TGF-β and Activin signaling are up-regulated in a number of key stromal rich cancers, indicating that they are both active and driving CAF induction in these tumors.

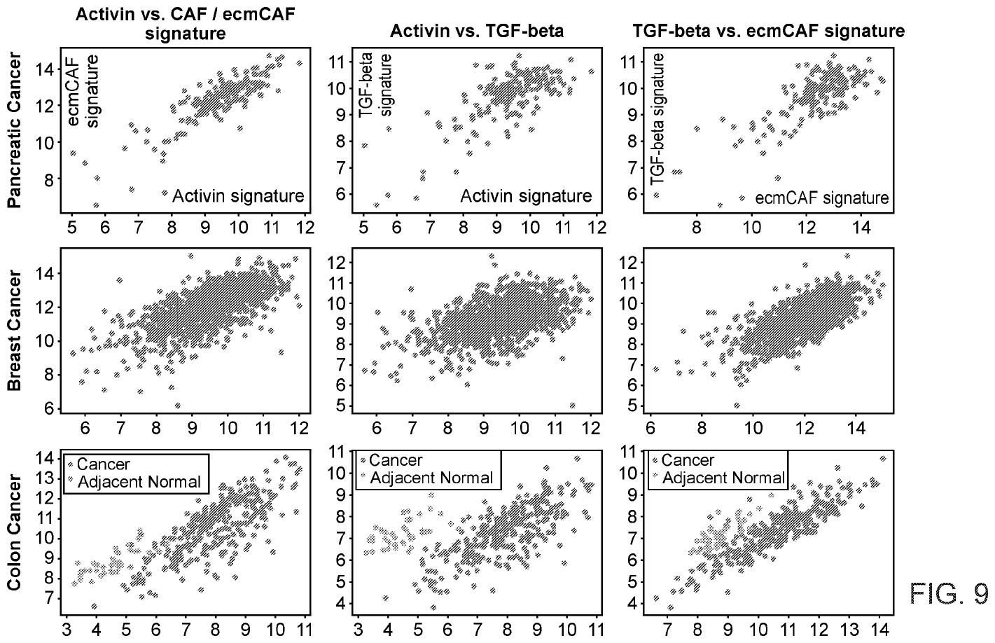

FIG. 9 Activin, TGF-β, and expression of an ecmCAF gene signature are tightly co-regulated across TCGA patient samples in key stromal rich cancers. To analyze how tightly co-regulated Activin, TGF-β and ecmCAF genes are between different tumor samples from stromal rich cancers, an analysis of how correlated the mRNA expression profiles are between these three gene signatures was performed in breast cancer, colorectal cancer, and pancreatic cancer. Scatter plots are shown comparing the natural log of the mean mRNA expression level score between the different signatures. In all cases the P value is very highly significant, a breakdown of R-values is given in Example 3. In Colon Cancer, mean expression level values are also plotted for adjacent tissue, since Activin and TGF-β levels are exceptionally dysregulated in this cancer type, indeed almost perfect separation between adjacent and tumor tissue can be achieved with these two genes alone. Taken together this diagram shows that an exceptionally high-level of co-regulation between these two genes suggests a level of cross-talk or co-activation exists between them.

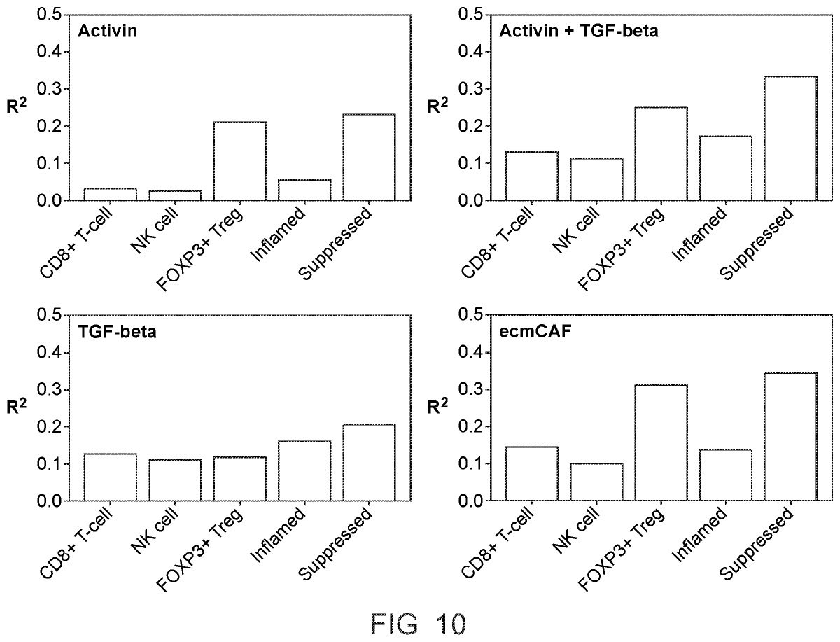

FIG. 10 Value of Activin, TGF-β, Activin/TGF-β, and ecmCAF gene expression levels in predicting immune-cell infiltration signatures across all TCGA primary tumors. Matrix were taken giving Activin levels for all TCGA samples (INHBA, INHBB), TGF-β levels for all TCGA samples (TGFB1, TGFB2, TGFB3), Activin plus TGF-β levels for all TCGA samples (INHBA, INHBB, TGFB1, TGFB2, TGFB3) and ecm CAF gene levels for all samples (COL1A1, COL1A2, LRRC15, POSTN, FAP, SERPINE1). Linear regression models were trained to predict the averaged gene signatures value of a sample for different immune infiltrates. Namely, CD8+ T-cells (CD8A, CD8B), NK Cells (KLRD1, KLRC1, KLRC2, KLRC3, GNLY KLRG1), and Tregs (FOXP3) were considered alongside broader inflamed (GZMM, GZMK, IFNG, GZMB, KLRD1, KLRC1, KLRC2, KLRC3, GNLY, KLRG1) and immunosuppressed (FOXP3, CTLA4, TNFRSF18, CD274, TIGIT) tissue signatures. R2 values we derived from each model and plotted to give an indication of how predictive any given gene signature is of a specific immune-infiltrate or tissue state. Overall this shows the value activin and TGF-β in predicting immune-infiltrates, notably immune-suppressive environments, suggesting these cytokines play a role in immuno-supression. That the combined score is greater than either cytokine alone, and closely aligns with the ecmCAF score further supports the value in targeting both simultaneously, and suggests they are likely tightly linked to regulation of the ecmCAF signature.

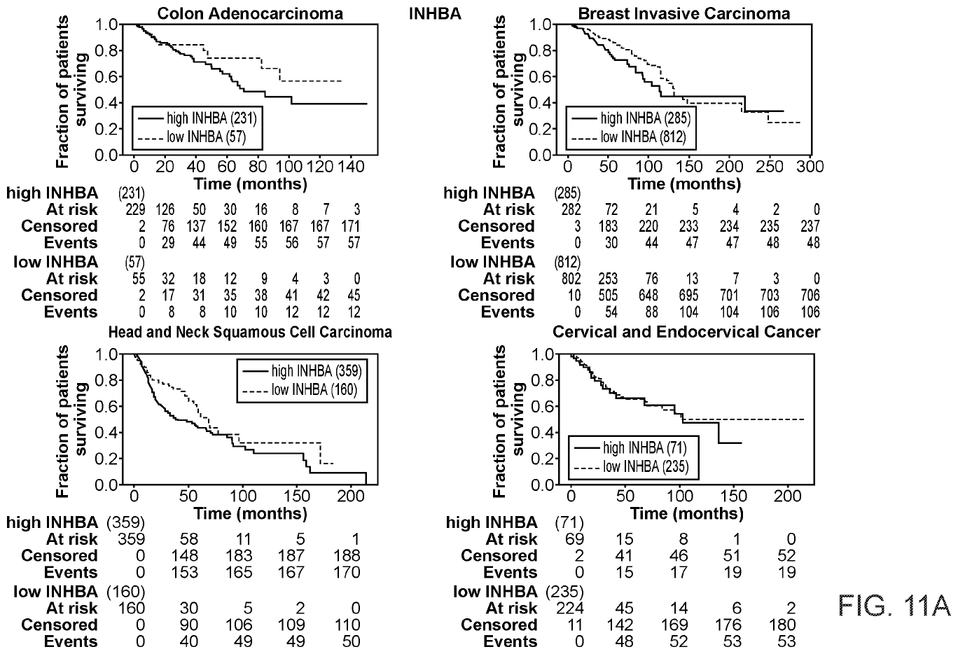

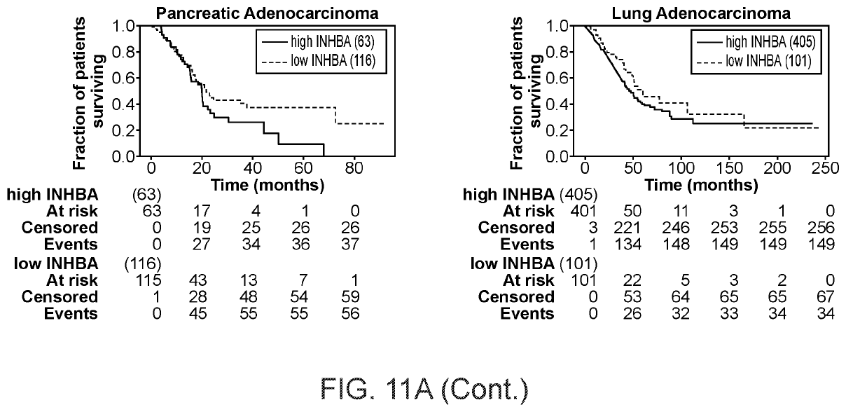

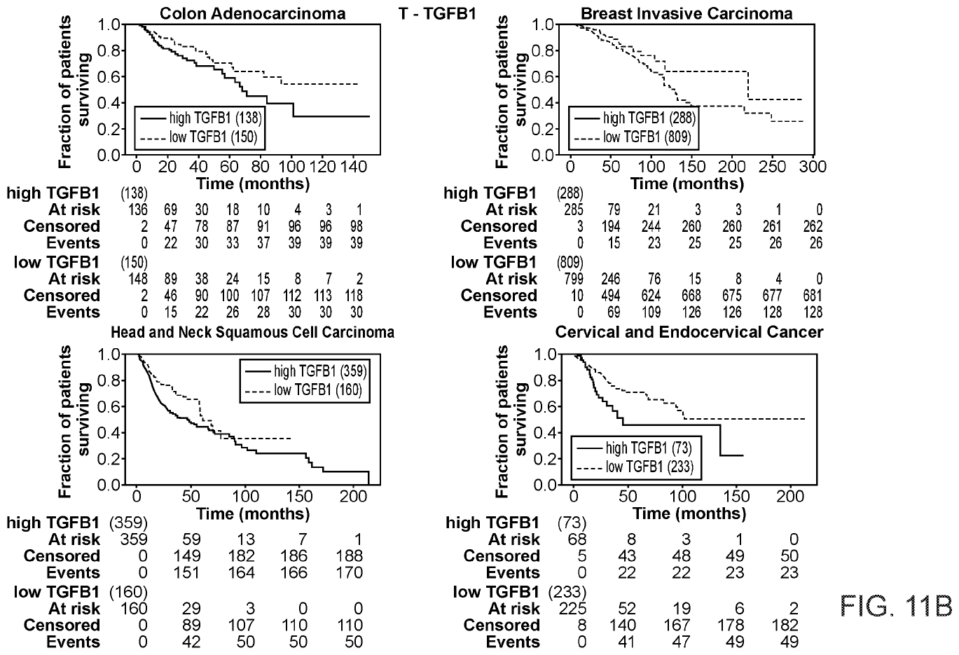

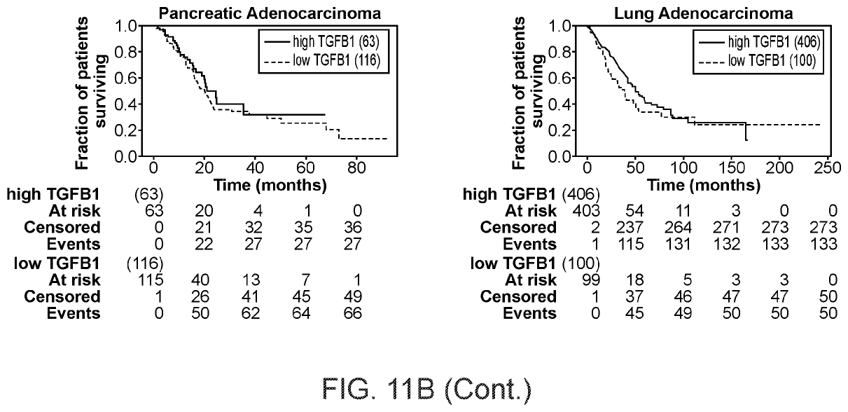

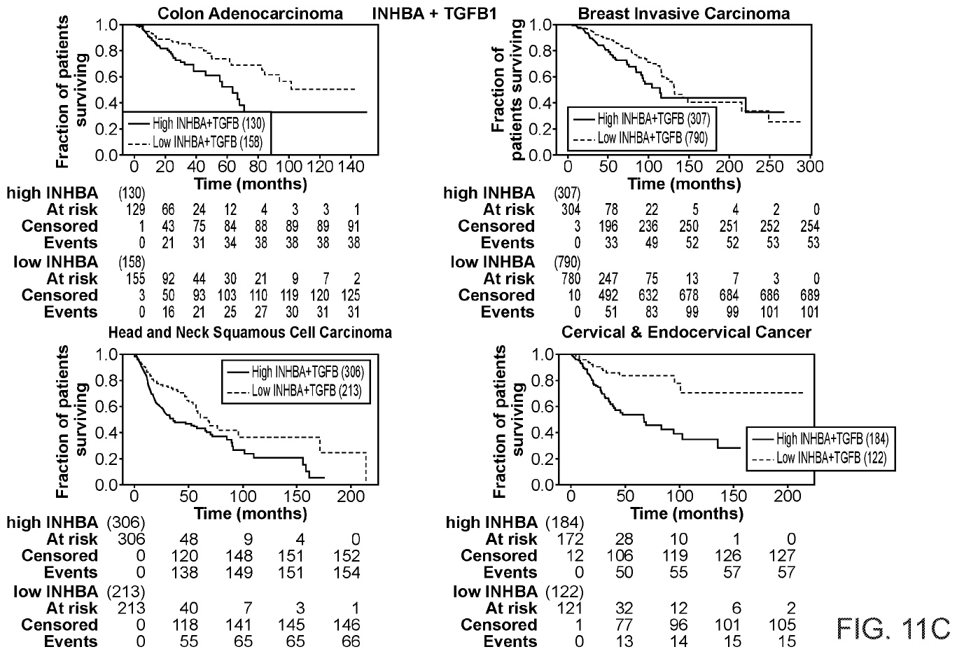

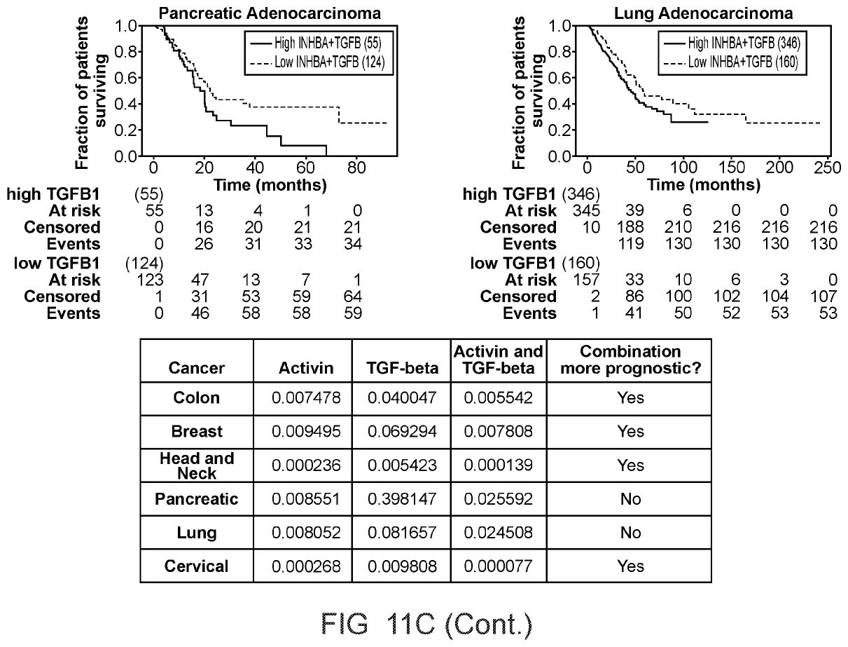

FIG. 11 Survival Curves for INHBA, TGFB1, and averaged INHBA/TGFB1 demonstrate the prognostic value of Activin and TGF-β mRNA expression levels in tumors. Survival curves were plotted between patients with tumors expressing high and low levels of: A) INHBA; B), TGFB1; and C) averaged INHBA/TGFB1 for a number of traditionally stromal rich, namely Breast, Colorectal, Pancreatic, Head and Neck, Cervical, and Lung cancers. INHBA and TGFB1 were chosen specifically as these are typically the highest expressed isoforms in tumor samples, and selection of a limited number of genes simplifies analysis and interpretation of results. Cut-offs between high and low expressors were based on TCGA defined cut-offs, that seek to maximize separation at the 95% cut-off interval. D) P-values were calculated based on the Hazard Ratio, between patient groups expressing high or low levels of the gene of interest. In the majority of cases a cut-off defined from an average of INHBA and TGFB1 levels improves upon a cut-off defined by mRNA expression levels of either gene alone. Again this suggests expression of both genes drives cancer, and in the absence of high-level of one gene, high-levels of another gene drive disease progression, resulting in their collective prognostic value.

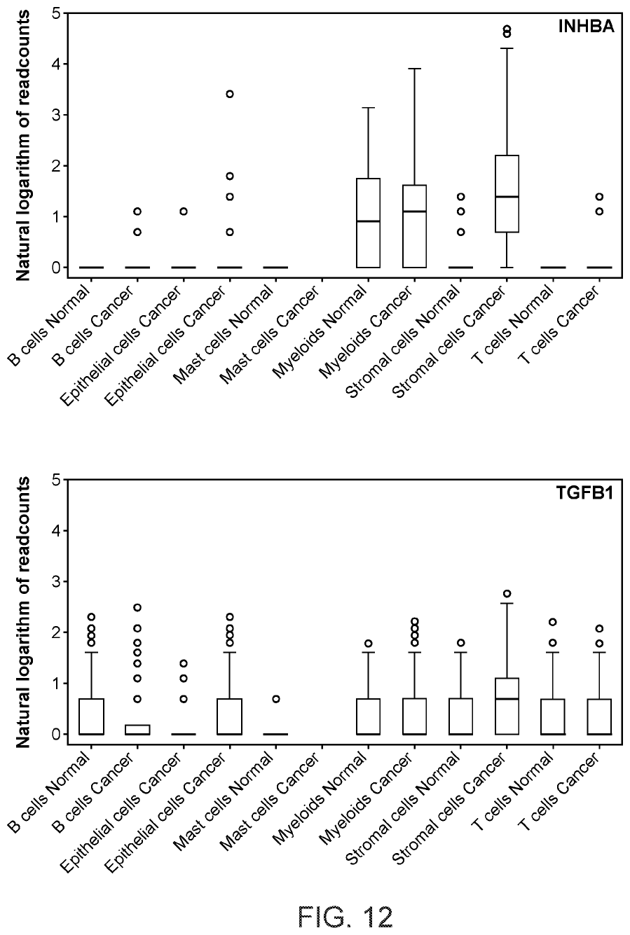

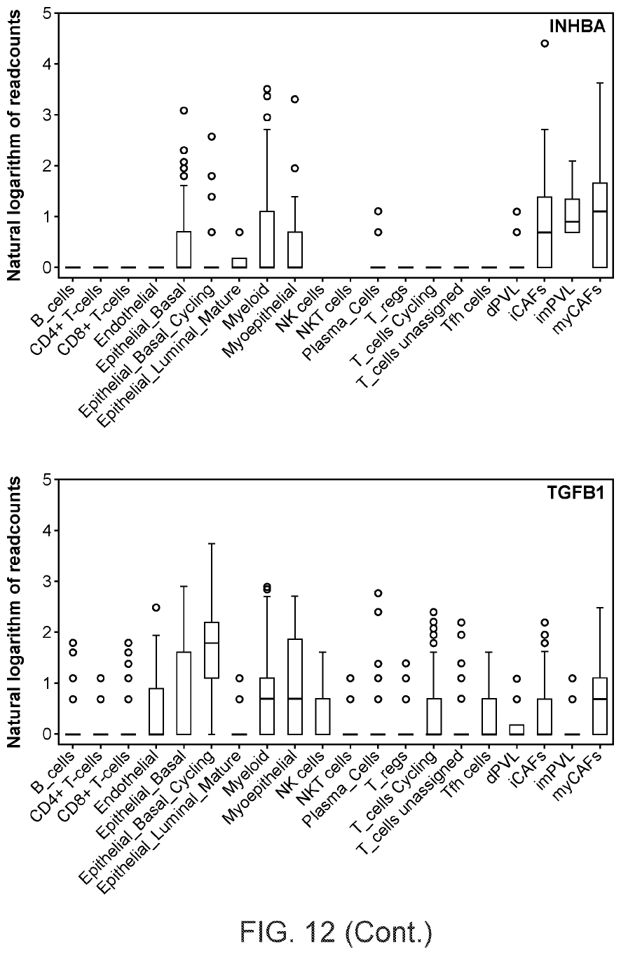

FIG. 12 Single cell expression of TGFB1 and INHBA levels in colorectal cancer and Triple Negative Breast Cancer (TNBC). In seeking to understand the cell-types expressing Activin and TGF-β in the Tumor Microenvironment and analysis of two public scRNA datasets was performed. Gene expression levels of each cytokine were averaged across cell-types defined by Author Annotations for each data set (Lee et al. 2020; Wu et al. 2020). TGF-β expression localizes to a broad set of cell-types, and is consistently up-regulated in cancer. Acitivn meanwhile localizes to CAFs and Myeloid/MAcrophages associated with the tumor. That we show in other examples that both Activin and TGF-β are able to induce a CAF like phenotype in fibroblasts suggests that whilst both proteins contribute to the induction of a CAF like phenotype in the tumor microenvironment, TGF-β is likely coming from many cells, but autocrine feedback either directly from CAFs or from nearby tumor associated macrophages further fuels CAF induction and maintenance through Activin signaling. Together this further demonstrates the need to target both Cytokines in the treatment of cancer.

### DETAILED DESCRIPTION

### Definitions

For purposes of interpreting this specification, the following definitions will apply, and whenever appropriate, terms used in the singular will also include the plural and vice versa. In the event that any definition set forth conflicts with any document incorporated herein by reference, the definition set forth below shall control. Unless defined otherwise, all technical and scientific terms used herein have the same meaning as commonly understood by one of ordinary skill in the art to which the disclosure pertains.

“About” as used herein when referring to a measurable value such as an amount, a temporal duration, and the like, is meant to encompass variations of ±20% or ±10%, more preferably ±5%, even more preferably +1%, and still more preferably ±0.1% from the specified value, as such variations are appropriate to perform the disclosed methods.

The terms “cancer” and “cancerous” refer to or describe the physiological condition in mammals that is typically characterized by unregulated cell growth. Examples of cancer include, but are not limited to, carcinoma, lymphoma, blastoma, sarcoma, and leukemia or lymphoid malignancies.

The term “tumor” as used herein, refers to all neoplastic cell growth and proliferation, whether malignant or benign, and all pre-cancerous and cancerous cells and tissues. In some embodiments, a “tumor” is a “cancerous tumor” and comprises one or more cancerous cells. Therefore, in some embodiments, the term “cancer” is equivalent to the term “tumor.”

The term “tumor stroma” or “stroma” as used herein, refers generally to the local tumor growing environment and comprises (1) nonmalignant cells of the tumor such as CAFs, specialized mesenchymal cell types distinctive to each tissue environment, innate and adaptive immune cells, and vasculature with endothelial cells and pericytes, and (2) the extracellular matrix (ECM) consisting of structural proteins (collagen and elastin), specialized proteins (e.g., fibrilin, fibronectin, and elastin), and proteoglycans (see e.g., Bremnes, et al. in J Thorac Oncol. (2011) 6: 209-217). Although the stromal cells are not themselves malignant, the tumor stroma is generally recognized as a major factor influencing the growth of cancer.

The term “cancer associated fibroblasts” or “CAFs” as used herein refers to activated fibroblastic cells in the tumor microenvironment (TME) of solid cancers that have a phenotype, function, or location distinct from normal, quiescent fibroblasts. CAFs may promote tumorigenic features, typically by initiating remodeling of the extracellular matrix or by secreting cytokines to one of the main components of tumor stroma. Thus, CAFs play essential roles in the TME, including in remodeling of the extracellular matrix, metastasis, and inflammation.

The term “stromal CAF enriched cancer” or “a solid tumor enriched in cancer fibroblasts” as used herein, refers to solid tumors enriched in cancer associated fibroblasts (CAFs). CAFs are associated with formation and growth of cancer. Typically, stromal CAF enriched cancers include, but are not limited to, tumors found in breast, pancreatic, and colorectal cancers.

The term “fibrotic disease” or “fibrosis” or “fibrotic scarring” as used herein, refers to any pathological condition resulting from an accumulation of excess fibrous tissue through either overproduction or insufficient degradation of extracellular matrix. The term “fibrotic disease” or “fibrotic condition” as used herein refers to any disease, condition, or disorder that is associated with fibrosis. The disease, condition, or disorder may be characterized by, caused by, or otherwise associated with fibrosis, either directly or indirectly. Exemplary fibrotic conditions include scarring, such as scarring of the skin (e.g., hypertrophic scarring and keloid scarring, among others) and internal tissues or organs. Fibrotic diseases represent a major cause of morbidity and mortality worldwide. Fibrotic disease can affect many organs, including liver, bone marrow, lung, kidney, GI tract, skin, eye, and endomyocardium, leading eventually to organ failure.

The term “transforming growth factor beta receptor family” or “TGFBR family” as used herein, refers to a family of serine/threonine kinase receptors involved in the TGF-β signaling pathway (see e.g., Heldin and Moustakas and (2016) Cold Spring Harb Perspect Biol August 2016). These receptors bind growth factor and cytokine signaling proteins of the TGF-β family such as e.g., TGFβs (TGF-β1, TGF-β2, TGF-β3), bone morphogenetic proteins (BMPs), growth differentiation factors (GDFs), activin and inhibin, myostatin, anti-Müllerian hormone (AMH), NODAL, etc. Thus, as used herein, an “activin receptor” or “activin receptor complex” is a “transforming growth factor beta receptor family” or “TGFBR family” member.

Three TGFβ superfamily receptors specific for TGF-β, can be distinguished by their structural and functional properties. TGFBR1 (ALK5) and TGFBR2 have similar ligand-binding affinities and can be distinguished from each other only by peptide mapping. Both TGFBR1 and TGFBR2 have a high affinity for TGF-β1 and low affinity for TGF-β2. TGFBR3 (β-glycan) has a high affinity for both homodimeric TGF-β1 and TGF-β2 and in addition, the heterodimer TGF-β1.2. The TGFβ receptors also bind TGF-β3.

The transforming growth factor 3 (TGF-β) family of cytokine genes has 33 human members, encoding TGF-β isoforms, bone morphogenetic proteins (BMPs), growth and differentiation factors (GDFs), activins, inhibins, nodal, and anti-Mullerian hormone (AMH) (see e.g., Rik Derynck and Kōhei Miyazono in Cold Spring Harbor Laboratory Press, (2008), “TGF-β Family”; Masato Morikawa et al. in Cold Spring Harb. Perspect. Biol. 8.5 (2016): a021873; Aristidis Moustakas and Carl-Henrik Heldin in Development 136.22 (2009): 3699-3714; Joan Massaguè in Nat. Rev. Mol. Cell Biol. 13.10 (2012): 616-30; Lalage M. Wakefield and Caroline S. Hill, in Nat. Rev. Cancer 13.5 (2013): 328-41). The family members are dimeric molecules, which in most cases are stabilized by a disulfide bond. TGF-β family members are synthesized as large precursors that need to be cleaved to liberate the carboxy-terminally located, active molecule.

The term “TGF-β,” “TGF beta,”, “TGF-beta”, “TGFB,” “transforming growth factor beta,” “TGF-β ligand” are used interchangeably herein to refer to a multifunctional cytokine belonging to the transforming growth factor β family (or superfamily). TGF-β regulates a wide variety of biological processes including cell proliferation and differentiation, migration and adhesion, extracellular matrix modification including tumor stroma and immunosuppression, angiogenesis and desmoplasia. Deregulation of TGF-β leads to pathological conditions, such as e.g., birth defects, cancer, chronic inflammation, and autoimmune and fibrotic diseases.

Mammalian TGF-β includes three different mammalian isoforms (TGF-β1, TGF-β2, TGF-β3). The three known isoforms are initially translated as a pro-peptide. After cleavage, the mature C-terminal end remains associated with the N-terminus (called the latency associated peptide or LAP), forming the small latent complex (SLC), which is secreted from the cell. The inability of the SLC to bind to TGFBR2 prevents receptor engagement. Activation by dissociation of the N- and C-termini occurs by one of several mechanisms, including proteolytic cleavage, acidic pH, or integrin structural alterations (see Erin C. Connolly et al. in Int. J. Biol. Sci. 8.7 (2012): 964-78). Thus, the term “TGF-β” or “transforming growth factor-beta” refers to the family of molecules described that have either the full-length, native amino acid sequence of any of the human TGF-β isoforms, including the latent forms (“latent TGF-β”) and associated or unassociated complex of precursors and mature TGF-β (see Table 1, Protein sequences). However, as used herein, unless otherwise indicated, the term TGF-β as used herein, typically refers to human TGF-β1 which has an amino acid sequence that is at least about 75%, at least about 80%, at least about 85%, at least about 90%, at least about 95%, at least about 99% identical to SEQ ID NO: 2 (P01137 [279-390]), that represents a mature form of TGF-β covering aa positions [279-390] of the pro-protein in SEQ ID NO: 1 (P01137). TGF-β is a ligand of TGF-β receptors (TGFBR).

The term “antagonist” or “inhibitor”, as used herein, refers in the broadest sense, to a substance that prevents, blocks, inhibits, neutralizes, or reduces a biological activity or effect of another molecule, such as a receptor or ligand. Suitable antagonist molecules specifically include antagonist antibodies or antibody fragments, fragments or amino acid sequence variants of native polypeptides, peptides, antisense oligonucleotides, small organic molecules, etc. Methods for identifying antagonists of a polypeptide, may comprise contacting the polypeptide with a candidate antagonist molecule and measuring a detectable change in one or more biological activities normally associated with the polypeptide.

Thus, the term “TGF-β inhibitor,” “TGF-β antagonist,” or “TGF-β blocker” as used herein, refers to any substance that inhibits the activity of TGF-β. For example by binding TGF-β or TGFBR to block the binding of TGF-β to TGFBR complexes. In some embodiments a “TGF-β inhibitor” is a small molecule. In some embodiments a “TGF-β inhibitor” is a peptidic inhibitor. In still other embodiments, a “TGF-β inhibitor” is an antibody or antibody fragment, that inhibits the activity of TGF-β. In some embodiments, a “TGF-β inhibitor” is an agent that binds and blocks the ability of TGF-β to bind to TGF-β receptor complexes.

The term “activin” as used herein, refers to any native activin from any vertebrate source, including mammals such as primates (e.g., humans, primates, and rodents (e.g., mice and rats), unless otherwise indicated. The activin molecule is also referred to as inhibin beta-1, Follicle-Stimulating Hormone-Releasing Protein (FRP), FSH-Releasing Protein, FSH-Releasing Factor, Erythroid Differentiation Factor (EDF) (see Table 1 for sequence IDs). Human activin A is encoded by the nucleotide sequence corresponding to activin INHBA isoform, which is initially translated to form a precursor protein activin βA that consists of a prodomain and a mature domain (Namwanje & Brown, Cold Spring Harb Perspect Biol (2016)) (SEQ ID NO: 9 (P08476) Table 1). This activin βA precursor protein then forms a homodimer with another activin βA (“latent form”) and is processed through pro-protein cleavage to form activin A (“mature form,” SEQ ID NO: 10). Human activin B is encoded by the nucleotide sequence corresponding to activin INHBB isoform. It also is initially transcribed as a precursor protein, activin βB, consisting of a prodomain and a mature domain (SEQ ID NO: 11 (P09529) Table 1). Two activin βB monomers dimerize (“latent form”), then undergo cleavage to form activin B (“mature form,” SEQ ID NO: 12). Human activin AB is a heterodimer consisting of one activin βA monomer and one activin OB monomer. Activin is a ligand of the TGF-β receptors (TGFBR).

Thus, the term “activin” encompasses “full-length,” unprocessed activin as well as any form of activin that results from processing in the cell. The term “activin” encompasses both latent and mature activin unless a specificity is indicated. The term also encompasses naturally occurring variants of activin, e.g., splice variants, allelic variants and isoforms. The activin polypeptides described herein may be isolated from a variety of sources, such as from human tissue types or from another source, or prepared by recombinant or synthetic methods. A “native sequence activin polypeptide” comprises a polypeptide having the same amino acid sequence as the corresponding activin polypeptide derived from nature. Such native sequence activin polypeptides can be isolated from nature or can be produced by recombinant or synthetic means. The term “native sequence activin polypeptide” specifically encompasses naturally-occurring truncated or secreted forms of the specific activin polypeptide (e.g., an extracellular domain sequence), naturally-occurring variant forms (e.g., alternatively spliced forms) and naturally-occurring allelic variants of the polypeptide. In some embodiments, the term “activin” refers to activin A (INHBA). In some embodiments, the term “activin” refers to activin A and/or activin B (INHBB) and/or activin AB.

The term “Activin inhibitor” or “activin inhibitor” refers to any substance that inhibits the activity of activin A and/or activin B. In some embodiments a “activin inhibitor” is a small molecule. In some embodiments an “activin inhibitor” is a peptidic inhibitor e.g., a protein, or peptide. In some embodiments an “activin inhibitor” is an antibody inhibitor e.g., an antibody or antibody fragment, that inhibits the activity of Activin. In some embodiments, a “activin inhibitor” is an agent that binds and blocks the ability of Activin to bind to TGF-β receptor complexes. In some embodiments, the Activin inhibitor is an anti-activin antibody disclosed in co-pending International Application No. PCT/US2021/065079, the disclosure of which is expressly incorporated by reference herein.

The term “transforming growth factor beta signaling pathway” or “TGF-β signaling pathway” as used herein, refers to a cytokine signaling pathway involved in many cellular processes in both the adult organism and the developing embryo including, but not limited to cell growth, cell differentiation, apoptosis, cellular homeostasis and other cellular functions. Thus, the TGFβ signaling pathway regulates a wide range of cellular processes. TGFβ superfamily ligands, which include TGF-β and activin, bind to a TGF-β type II receptor (TGFBR2), which recruits and phosphorylates a type I receptor (TGFBR1). The type I receptor then phosphorylates receptor-regulated SMADs (R-SMADs) which can then bind the coSMAD, SMAD4. R-SMAD/coSMAD complexes accumulate in the nucleus where they act as transcription factors and participate in the regulation of target gene expression.

Thus, as used herein, the term “TGF-β signaling” refers to the endogenous signal transduction cascade by which TGF-β and other TGF-β receptor ligands (e.g. activin) potentiates the intracellular activity of the TGF-β receptor so as to effect one or more biological responses. “TGF-β signaling” encompasses the TGF-β or activin-mediated stimulation of a TGF-β receptor r TGF-β receptor family member and concomitant phosphorylation and activation of receptor-associated Smad proteins. TGF-β signaling includes the translocation of one or more Smad transcription factors to the nucleus, for example, by way of an interaction between a Smad protein and nucleoporins. TGF-β signaling encompasses the release of one or more Smad protein from Smad Anchor for Receptor Activation (SARA), which sequesters Smad proteins in the cytoplasm and prevents their translocation into the nucleus.

As used herein, the term “immune checkpoints” refers to inhibitory pathways utilized by the immune system to maintain tolerance to self and limit the magnitude of the immune response in tissues to minimize inflammation-associated damage. As used herein, the term “Immune checkpoint proteins” typically refers to receptors such as PD-1 and CTLA-4 or their respective ligands, the result of whose binding induces an inhibitory immune signal, decreasing T cell activation. As used herein, the term “Immune checkpoint inhibitors” refers to agents, typically therapeutic antibodies, that bind to an inhibitory immune checkpoint and blocks its activity. Examples of immune checkpoint inhibitors are the anti-PD-1 antibodies nivolumab and pembrolizumab and the anti-CTLA-4 antibodies ipilimumab and tremelimumab. Additional examples of checkpoint inhibitors include antibodies to the receptors LAG3, TIGIT, and TIM-3. As used herein, the term “Costimulatory Receptors” may refer to proteins that, following initial T cell activation, may induce additional activation resulting in responses such as cell proliferation, cytokine secretion, or differentiation. As used herein, the term “costimulatory agonist” refers to an antibody to a costimulatory receptor such as CD28, ICOS, 4-1BB, OX40, GITR whose binding induces similar activation of the receptor as its natural ligand. Examples of checkpoint inhibitors and costimulatory receptor agonists in clinical development are in Table 2.

The term “antibody,” as used herein, refers to an immunoglobulin molecule which specifically binds with an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present disclosure may exist in a variety of forms including, for example, polyclonal antibodies, monoclonal antibodies, Fv, Fab and F(ab)2, as well as single chain antibodies and humanized antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426; For a review of sFv, see, e.g., Pluckthun in The Pharmacology ofMonoclonal Antibodies, vol. 113, Rosenburg and Moore eds., Springer-Verlag, New York, pp. 269-315 (1994)).

The term “antibody fragment” refers to a portion of an intact antibody and refers to the antigenic determining variable regions of an intact antibody Examples of antibody fragments include Fab, Fab′, F(ab′)2, and Fv fragments; diabodies; linear antibodies (see U.S. Pat. No. 5,641,870, Example 2; Zapata et al., Protein Eng. 8(10): 1057-62 (1995)); single-chain antibody molecules; and multispecific antibodies formed from antibody fragments. In one embodiment, an antibody fragment comprises an antigen binding site of the intact antibody and thus retains the ability to bind antigen.

An “antibody heavy chain,” as used herein, refers to the larger of the two types of polypeptide chains present in antibody molecules in their naturally occurring conformations.

An “antibody light chain,” as used herein, refers to the smaller of the two types of polypeptide chains present in antibody molecules in their naturally occurring conformations. lc and a, light chains refer to the two major antibody light chain isotypes.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “antigen” or “Ag” as used herein is defined as a molecule that provokes an immune response. This immune response may involve either antibody production, or the activation of specific immunologically-competent cells, or both. The skilled artisan will understand that any macromolecule, including proteins or peptides, can serve as an antigen. Furthermore, antigens can be derived from recombinant or genomic DNA. A skilled artisan will understand that any DNA that comprises a nucleotide sequence or a partial nucleotide sequence encoding a protein that elicits an immune response therefore encodes an “antigen” as that term is used herein. Furthermore, one skilled in the art will understand that an antigen need not be encoded solely by a full-length nucleotide sequence of a gene. It is readily apparent that the present disclosure includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated, synthesized, or can be derived from a biological sample. Such a biological sample can include, but is not limited to a tissue sample, a tumor sample, a cell or a biological fluid.

The term “epitope” includes any protein determinant, lipid or carbohydrate determinant capable of specific binding to an immunoglobulin or T-cell receptor. Epitopic determinants usually consist of active surface groupings of molecules such as amino acids, lipids or sugar side chains and usually have specific three-dimensional structural characteristics, as well as specific charge characteristics. An antibody is said to specifically bind an antigen when the equilibrium dissociation constant (KD) is in the range of 10⁻⁶-10⁻¹²M

The term “therapeutically effective amount” as used herein, is meant an amount that produces the desired effect for which it is administered by the researcher, veterinarian, medical doctor or other clinician. In some embodiments, the term refers to an amount that is sufficient, when administered to a population suffering from or susceptible to a disease, disorder, and/or condition in accordance with a therapeutic dosing regimen, to treat the disease, disorder, and/or condition. In some embodiments, a therapeutically effective amount is one that reduces the incidence and/or severity of, and/or delays onset of, one or more symptoms of the disease, disorder, and/or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment. In some embodiments, reference to a therapeutically effective amount may be a reference to an amount as measured in one or more specific tissues (e.g., a tissue affected by the disease, disorder or condition) or fluids (e.g., blood, saliva, serum, sweat, tears, urine, etc.). Those of ordinary skill in the art will appreciate that, in some embodiments, a therapeutically effective amount of a particular agent or therapy may be formulated and/or administered in a single dose. In some embodiments, a therapeutically effective agent may be formulated and/or administered in a plurality of doses, for example, as part of a dosing regimen.

The term “therapeutic regimen”, as used herein, refers to a dosing regimen whose administration across a relevant population is or is expected to be correlated with a desired or beneficial therapeutic outcome.

As used herein, the term “treatment” (also “treat” or “treating”), in its broadest sense, refers to any administration of a substance (e.g., provided compositions) that partially or completely alleviates, ameliorates, relives, inhibits, delays onset of, reduces severity of, and/or reduces incidence of one or more symptoms, features, and/or causes of a particular disease, disorder, and/or condition. In some embodiments, such treatment may be administered to a subject who does not exhibit signs of the relevant disease, disorder and/or condition and/or of a subject who exhibits only early signs of the disease, disorder, and/or condition. Alternatively or additionally, in some embodiments, treatment may be administered to a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

As used herein, the term “administration” refers to the administration of a composition to a subject or system (e.g., to a cell, organ, tissue, organism, or relevant component or set of components thereof). Those of ordinary skill will appreciate that route of administration may vary depending, for example, on the subject or system to which the composition is being administered, the nature of the composition, the purpose of the administration, etc. For example, in certain embodiments, administration to an animal subject (e.g., to a human) may be bronchial (including by bronchial instillation), buccal, enteral, interdermal, intra-arterial, intradermal, intragastric, intramedullary, intramuscular, intranasal, intraperitoneal, intrathecal, intravenous, intraventricular, mucosal, nasal, oral, rectal, subcutaneous, sublingual, topical, tracheal (including by intratracheal instillation), transdermal, vaginal and/or vitreal. In some embodiments, administration may involve intermittent dosing. In some embodiments, administration may involve continuous dosing (e.g., perfusion) for at least a selected period of time.

As used herein, the term “combination therapy” refers to those situations in which a subject is simultaneously exposed to two or more therapeutic regimens or modalities (e.g., to two or more therapeutic agents). In some embodiments, two or more regimens or modalities are administered or applied simultaneously (e.g., one or more individual doses of each of two or more agents, may be administered at substantially the same time); in some embodiments, such regimens or modalities may be administered sequentially (e.g., at least a first dose of a first agent is administered prior to at least a first dose of a second agent); in some embodiments, such regimens or modalities such that individual doses or applications overlap.

As used herein, the term “refractory” refers to any subject or condition that does not respond with an expected clinical efficacy following the administration of provided compositions as normally observed by practicing medical personnel.

As used herein, a “response” to treatment may refer to any beneficial alteration in a subject's condition that occurs as a result of or correlates with treatment. Such alteration may include stabilization of the condition (e.g., prevention of deterioration that would have taken place in the absence of the treatment), amelioration of symptoms of the condition, and/or improvement in the prospects for cure of the condition, etc. It may refer to a subject's response or to a tumor's response. Tumor or subject response may be measured according to a wide variety of criteria, including clinical criteria and objective criteria. Techniques for assessing response include, but are not limited to, clinical examination, positron emission tomatography, chest X-ray CT scan, MRI, ultrasound, endoscopy, laparoscopy, presence or level of tumor markers in a sample obtained from a subject, cytology, and/or histology. Many of these techniques attempt to determine the size of a tumor or otherwise determine the total tumor burden. Methods and guidelines for assessing response to treatment are discussed in Therasse et. al., “New guidelines to evaluate the response to treatment in solid tumors”, European Organization for Research and Treatment of Cancer, National Cancer Institute of the United States, National Cancer Institute of Canada, J. Natl. Cancer Inst., 2000, 92(3):205-216. The exact response criteria can be selected in any appropriate manner, provided that when comparing groups of tumors and/or patients, the groups to be compared are assessed based on the same or comparable criteria for determining response rate. One of ordinary skill in the art will be able to select appropriate criteria.

The term “pharmaceutically acceptable”, as used herein, refers to a material, including but not limited, to a salt, carrier or diluent, which does not abrogate the biological activity or properties of the compound, and is relatively nontoxic, i.e., the material may be administered to an individual without causing undesirable biological effects or interacting in a deleterious manner with any of the components of the composition in which it is contained.

As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

The terms “patient,” “subject,” “individual,” and the like are used interchangeably herein, and refer to any animal, amenable to the methods described herein. In certain non-limiting embodiments, the patient, subject or individual is a human.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

In some embodiments, specific binding can be characterized by an equilibrium dissociation constant of at least about 1×10⁻⁸ M or less (e.g., a smaller K_{D} denotes a tighter binding). Methods for determining whether two molecules specifically bind are well known in the art and include, for example, equilibrium dialysis, surface plasmon resonance, and the like. Moreover, multi-specific antibodies that bind to a first antigen and one or more additional antigens or a bispecific antibody that binds to two different regions of an antigen are nonetheless considered antibodies that “specifically bind,” as used herein.

Ranges: throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

The term “substantial identity” or “substantially identical,” when referring to a nucleic acid or fragment thereof, indicates that, when optimally aligned with another nucleic acid (or the complementary strand of the other nucleic acid), there is nucleotide sequence identity in %, for example, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, or 100% of the nucleotide bases, as measured by any well-known algorithm of sequence identity, such as FASTA, BLAST or GAP, as discussed below. A nucleic acid molecule having substantial identity to a reference nucleic acid molecule may, in certain instances, encode a polypeptide having the same or substantially similar amino acid sequence as the polypeptide encoded by the reference nucleic acid molecule.

As applied to polypeptides, the term “substantial similarity” or “substantially similar” means that two peptide sequences, when optimally aligned, such as by the programs GAP or BESTFIT using default gap weights, share at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, or 100% sequence identity. In some aspects, residue positions, which are not identical, differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent or degree of similarity may be adjusted upwards to correct for the conservative nature of the substitution. Means for making this adjustment are well known to those of skill in the art. See, e.g., Pearson (1994) Methods Mol. Biol. 24: 307-331, which is herein incorporated by reference. Examples of groups of amino acids that have side chains with similar chemical properties include 1) aliphatic side chains: glycine, alanine, valine, leucine and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; 6) acidic side chains: aspartate and glutamate, and 7) sulfur-containing side chains: cysteine and methionine. Preferred conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al. (1992) Science 256: 1443 45, herein incorporated by reference. A “moderately conservative” replacement is any change having a nonnegative value in the PAM250 log-likelihood matrix.

Sequence identity and/or similarity for polypeptides is typically measured using sequence analysis software. Protein analysis software matches similar sequences using measures of similarity assigned to various substitutions, deletions and other modifications, including conservative amino acid substitutions. For instance, GCG software contains programs such as GAP and BESTFIT which can be used with default parameters to determine sequence homology or sequence identity between closely related polypeptides, such as homologous polypeptides from different species of organisms or between a wild type protein and a mutein thereof. See, e.g., GCG Version 6.1. Polypeptide sequences also can be compared using FASTA with default or recommended parameters; a program in GCG Version 6.1. FASTA (e.g., FASTA2 and FASTA3) provides alignments and percent sequence identity of the regions of the best overlap between the query and search sequences. Sequences also can be compared using the Smith-Waterman homology search algorithm using an affine gap search with a gap open penalty of 12 and a gap extension penalty of 2, BLOSUM matrix of 62. Another preferred algorithm when comparing a sequence disclosed herein to a database containing a large number of sequences from different organisms is the computer program BLAST, especially BLASTP or TBLASTN, using default parameters. See, e.g., Altschul et al. (1990) J. Mol. Biol. 215: 403-410 and (1997) Nucleic Acids Res. 25:3389-3402, each of which is herein incorporated by reference.

As used herein, terms such as “improve,” “increase,” “reduce,” etc., which necessarily imply a comparison, refer to a comparison with an appropriate comparable reference or standard. For example, in some embodiments, level and/or activity of an agent or marker of interest may be reduced under a set of conditions or circumstances of interest (e.g., after administration of therapy) as compared with its level and/or activity under a comparable set of conditions (e.g., prior to administration of the therapy or after administration of the therapy to an appropriate reference subject). In some embodiments, an appropriate reference may be a historical reference. In some embodiments, an appropriate reference may be an average, e.g., as may be observed within or across a relevant population.

The terms “predictive” and “prognostic” as used herein are also interchangeable. In one sense, the methods for prediction or prognostication are to allow the person practicing a predictive/prognostic method as disclosed herein to select patients that are deemed (usually in advance of treatment, but not necessarily) more likely to respond to treatment with an anti-cancer agent, preferably using a composition disclosed herein.

### I. Introduction

TGF-β is part of a wider group of homologous proteins, the TGF-β superfamily, that influence every aspect of human biology. For example, the bone morphogenic proteins, (BMPs) originally identified for their role in regulating bone formation and remodeling have now been implicated as key factors in a huge range of processes including, developmental patterning and growth regulation, immune-cell activation and skewing, maintenance of stem-cell niches, and as factors driving disease progression in cancer, and fibrosis (see e.g., Yigong Shi and Joan Massaguè, in Cell 113.6 (2003): 685-700; F. A. Millan et al. in Development 111.1 (1991): 131-43; J. H. Kehrl et al. in J. Exp. Med. 163.5 (1986): 1037-50; Julien C. Marie et al. in J. Exp. Med. 201.7 (2005): 1061-67; Linheng Li and William B. Neaves in Cancer Res. 66.9 (2006): 4553-57; Raghu Kalluri and Michael Zeisberg, in Nat. Rev. Cancer 6.5 (2006): 392-401; and Pierre-Jean Wipff et al., in J. Cell Biol. 179.6 (2007): 1311-23).

TGF-β plays a key role in immunity and wound repair. In immunity, TGF-β has anti-inflammatory properties, exemplified by the fact that a TGF-β knockout in mice leads to fatal early onset autoimmune disease (see e.g., H. Dang et al. in J. Immunol. 155.6 (1995): 3205-12; M. Christ et al. in J. Immunol. 153.5 (1994): 1936-46). In wound healing, TGF-β is perhaps the most intensely studied mechanism by which fibroblasts can be ‘activated’ into myofibroblasts (see Desmouliére et al., in Journal of Cell Biology, (1993)). Myofibroblasts, in turn, drive wound closure through generating contractile force, remodel the ECM to provide new matrix for cell-growth and support, and secrete a variety of factors that drive growth and repair in neighboring cells. Given the similarities between cancer and wound healing (see F. Grinnell in J. Cell Biol. 124.4 (1994): 401-4), it is unsurprising that CAFs also share many similarities at the molecular and functional level with myofibroblasts. TGF-β is also a key factor in driving CAF induction in the tumor microenvironment. TGF-β's role in inducing CAF activation and driving immune-suppression properties has thus made targeting this cytokine in cancer a focus point in drug-discovery.

There is now mounting evidence that TGF beta inhibition in murine preclinical models that mimic clinical settings where patients do not responded to immune checkpoint monotherapy. Namely, TGFb inhibition can be additive with immune checkpoint therapy in preclinical models (Mariathasan et al. 2018; Tauriello et al. 2018). In both of these instances anti TGF-β alone is not effective in reducing tumor volume but when looking at pharmacodynamic properties molecular changes are observed such as inhibition of SMAD protein phosphorylation and reduction in matrix genes associated with myofibroblasts. At the cellular level, TGF beta inhibition can reduce the number of suppressive macrophages as well as reprogramming the phenotype of the dominant fibroblast population to an interferon response cell type more amenable to checkpoint blockade. Indeed, when monotherapy TGF beta is combined with PD1/L1 blockade several complete responders are seen in one study (Mariathasan et al. 2018) while more prominent blockade is noted in a recent study by Grauel et al. in Nat. Commun. 11.1 (2020): 6315. Impressive as these data are, there are no evidence of long term remission/protection from additional tumor challenges and there are several tumors that escape treatment, suggesting the need for additional therapeutics.

Activin A is another member of the TGF-β superfamily of ligands and plays important roles in cell proliferation, wound healing and the immune response (see e.g., Ioannis Morianos et al. in Journal of Autoimmunity, (2019) 104.102314); Maria Antsiferova and Sabine Werner in J Cell Sci. 125.Pt 17 (2012): 3929-37). Activin A was initially identified as a regulator of follicle stimulating hormone (FSH) biosynthesis, where its activity enhances spermatogenesis and follicular development (see W. Vale et al. in Nature 321.6072 (1986): 776-79). Since then, activin A has been characterized in homeostasis as a key regulator of muscle mass and wound healing. In line with its role in wound healing, studies have also highlighted aberrant regulation of activin A signaling as a mediator of tumorigenesis, largely through its impact on the tumor stroma (see Jasmin Zessner-Spitzenberg et al. in Gene Rep 17 (2019) 100501; Jessica Bauer in Sci. Rep. 10.1 (2020): 50). Activin A levels are increased in multiple cancers, and its signaling in fibroblasts has been shown to induce activation and transition into a myofibroblast phenotype as shown by upregulation of markers such as αSMA, FAP, and increased expression of extracellular matrix proteins (see Xiaoting Li et al. in Dis. Markers 2019 (2019): 7275289). activin A has also been shown to have effects on immune cells, where its signaling through T cells and macrophages can skew them towards T-reg and M2 phenotypes that contribute to an immunosuppressive tumor microenvironment (see e.g, Kenji Ogawa et al. in J. Immunol. 177.10 (2006): 6787-94; Samuel Huber et al. in The Journal of Immunology, 2009,182 (8) 4633-4640).

Although there are many examples that highlight the role individual TGF-β superfamily members play in human biology, understanding how different members of this family interact to induce distinct cell-states and effects is severely limited. Accordingly, prior to the present disclosure, compositions and methods that target combinations TGF-β superfamily members as therapies for cancer and other disorders have been largely unexplored.

Fortunately, the present inventors have taken on the challenge and discovered combination therapies for successfully treating a number of diseases and disorders.

### II. General Methods

This disclosure utilizes routine techniques in the field of recombinant genetics, microbiology, cell biology, immunology, and biochemistry which are known to those of skill in the art. Basic texts disclosing the general terms in molecular biology and genetics include e.g., Lackie, Dictionary of Cell and Molecular Biology, Elsevier (5th ed. 2013). Basic texts disclosing methods in recombinant genetics and molecular biology include e.g., Sambrook et al, Molecular Cloning—A Laboratory Manual, Cold Spring Harbor Press 4th Edition (Cold Spring Harbor, N.Y. 2012) and Current Protocols in Molecular Biology Volumes 1-3, John Wiley & Sons, Inc. (1994-1998), Supplements 1-115 (1987-2016) and periodic updates. Basic texts disclosing the general methods and terms in biochemistry include e.g., Lehninger Principles of Biochemistry sixth edition, David L. Nelson and Michael M. Cox eds. W.H. Freeman (2012). Basic texts disclosing the general methods and terms immunology include Janeway's Immunobiology (Ninth Edition) by Kenneth M. Murphy and Casey Weaver (2017) Garland Science; Fundamental Immunology (Seventh Edition) by William E. Paul (2013) Lippincott, Williams and Wilkins; and Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426. Oligonucleotide Synthesis” (M. J. Gait, ed., 1984); “Animal Cell Culture” (R. I. Freshney, ed., 1987); “Methods in Enzymology” (Academic Press, Inc.); “PCR: The Polymerase Chain Reaction”, (Mullis et al., ed., 1994); “A Practical Guide to Molecular Cloning” (Perbal Bernard V., 1988); “Phage Display: A Laboratory Manual” (Barbas et al., 2001)

One skilled in the art will recognize many methods and materials similar or equivalent to those described herein, which could be used in the practice of the methods disclosed herein. Indeed, the compositions and method disclosed herein in no way limited to the methods and materials described.

### Cancer Associated Fibroblast (CAF) Enriched Cancers

In another aspect, the disclosure provides compositions and methods for treating solid tumors enriched in cancer fibroblasts e.g., pancreatic, breast, or colorectal cancers. Solid tumors enriched in cancer fibroblasts experience significant up-regulation of both TGF-β and activin at the mRNA and protein expression levels. Histologically, solid cancers such as these have a dense tumor stroma composed of CAFs, a rich extracellular matrix, and both myeloid cells and suppressed CD3+ T-cells.

Solid tumors enriched in cancer fibroblasts such as e.g., pancreatic, breast, or colorectal cancers can metastasize to locations distal to the site of the primary cancer. Accordingly, in one embodiment, the disclosure provides methods for treating metastasis of pancreatic, breast, or colorectal primary tumors that reside in the liver, bone, brain, or other organs. In another embodiment, administration of a combination treatment that utilizes activin and TGF-β inhibitors is further combined with a checkpoint inhibitor.

In another embodiment, the combination treatment with activin and TGF-β inhibitors is combined with surgery or existing standard of care agents such as chemotherapy regimens, radiation therapy, or treatment with one or more targeted agents such as the anti-angiogenic agent bevacizumab (Avastin) that is currently used to treat late stage colorectal and breast cancers.

In another aspect, the disclosure provides compositions and methods for blocking the transformation of normal tissue fibroblasts into CAFs. In one embodiment, the transformation of normal tissue fibroblasts into CAFs is blocked in vitro. Transformation of normal tissue fibroblasts into CAFs can be measured in vitro by any one of a number of measures including protein expression level increases in fibroblast activation protein (FAP), alpha-smooth muscle actin (αSMA), podoplanin (PDPN), and fibronectin as well as protein expression decrease in Endosialin (CD248), and morphological changes such as actin filament bundling, and SMAD2/3, or 4 nuclear localization.

Addition of recombinant TGF-β or activin to normal fibroblasts induces transformation of fibroblasts into CAFs. Blockade of either TGF-β or activin fails to prevent CAF transformation in the presence of the other ligand. Moreover, in co-cultures of cancer cells and fibroblasts wherein both TGF-β and activin are present, blockade of both TGF-β and activin results in more significant reduction in measure of CAF induction than either agent alone see e.g., Example 1. In another embodiment, in co-cultures of normal fibroblasts and cancer cells, blockade of both TGF-β and activin demonstrates anti-proliferative activity on fibroblasts and CAFs thus reducing the pro-tumor effects of the stroma. Accordingly, the disclosure provides methods for blocking or inhibiting CAF induction in solid cancers enriched in cancer fibroblasts.

### Fibrotic Disease

In another aspect, the disclosure provides compositions and methods for improving the efficacy of TGF-β inhibitors in fibrosis. Fibrotic diseases are a leading cause of death globally, and describe a variety of specific disease that share the common property of aberrant, chronic, fibroblast activation and extracellular matrix (ECM) remodeling that results in healthy normal tissue becoming stiff, dysfunctional, scar tissue. Major fibrotic disease indications include but are not limited to: Pulmonary Fibrosis (PF), Idiopathic Pulmonary Fibrosis (IPF), Liver Cirrhosis, Non-alcoholic steric hepatitis (NASH), Pulmonary Arterial Hypertension (PAH), Renal Fibrosis, Chronic Kidney Disease (CKD), Chronic Dermal Fibrosis, Scleroderma, Hypertrophic Scar, Cardiac Fibrosis, Hypertrophic Cardiomyopathy, and in some aspects Alzheimer's Disease and Inflammatory Bowel Disease.

In Fibrotic diseases, activated fibroblasts, termed myofibroblasts share many properties with CAFs. Indeed ECM producing, contractile CAFs, are often termed myofibroblastic CAFs or ‘myCAFs’ in literature, indicating their similarities. Accordingly, in some embodiments, therapeutic strategies that target CAFs are used to treat fibrotic diseases. Thus, in one embodiment, the efficacy of treatment for fibrotic diseases afforded by the selective inhibition of a specific TGF-β isoform is enhanced by simultaneously inhibiting activin. Enhanced efficacy reduces the dosage amount and thus provides effective treatment with a low level of toxicity.

### Peptidic Inhibitors/Antagonists

In some embodiments a “TGF-β inhibitor” or “activin inhibitor” is a peptidic inhibitor, e.g., a protein, such as e.g., a fusion protein, or a low-molecular weight peptide capable of inhibiting TGF-β signaling. Peptidic TGF-β or activin inhibitor typically comprises, consists, or consists essentially of a peptide or fusion protein. Thus, a TGF-β or activin inhibitor may comprise, consist of, or consist essentially of a TGF-β or activin inhibitor that is a peptide capable of binding TGF-β, activin, or a TGF-β receptor.

Binding of a peptide to TGF-β, activin, or a TGF-β receptor can be assessed by any method known in the art. For instance, using a protein binding assay known in the art, such as ELISA, fluorescence anisotropy or fluorescence polarization, and calorimetry, such as isothermal titration calorimetry (ITC). Binding of a peptide to TGF-β, activin, or a TGF-β receptor can also be assessed by observing a decrease in TGF-β signaling, for instance, due to binding of TGF-β to a TGF-β-binding peptide that is competitive with binding of TGF-β to a TGF-β receptor. Binding of a peptide, for instance, to TGF-β, activin, or a TGF-β receptor can be determined, for example, by observing peptide-mediated inhibition of TGF-β induced, Smad3-driven transcription. This can be measured, for example, using an in vitro reporter expression assay, such as an in vitro luciferase expression assay well known in the art. Exemplary peptidic inhibitors of TGF-β include e.g., ACVR2A, ACVR2A. Exemplary peptidic inhibitors of activin include e.g., Follistatin-Fc.

### Antibody Inhibitors/Antagonists

A “blocking” antibody or an “antagonist” antibody is one which inhibits or reduces biological activity of the antigen it binds. Preferred blocking antibodies or antagonist antibodies substantially or completely inhibit the biological activity of the antigen.

Therefore, in some embodiments, the TGF-β or activin inhibitor/antagonist is an antibody or antibody fragment that specifically binds to one or more isoforms of TGF-β or of activin; a TGF-β receptor or soluble fragment thereof, or an antibody or antibody fragment that specifically binds to one or more TGF-β receptors.

An antibody that “binds” an antigen or epitope of interest is one that binds the antigen or epitope with sufficient affinity that is measurably different from a non-specific interaction. Specific binding can be measured, for example, by determining binding of a molecule compared to binding of a control molecule, which generally is a molecule of similar structure that does not have binding activity.

An antibody that inhibits the growth of tumor cells is one that results in measurable growth inhibition of cancer cells. In one embodiment, an anti-activin antibody is capable of inhibiting the growth of cancer cells displaying the activin tumor epitope. Preferred growth inhibitory anti-activin antibodies inhibit growth of activin-expressing tumor cells by greater than 20%, preferably from about 20% to about 50%, and even more preferably, by greater than 50% (e.g., from about 50% to about 100%) as compared to the appropriate control, the control typically being tumor cells not treated with the antibody being tested.

Anti-activin antibodies may (i) inhibit the growth or proliferation of a cell to which they bind; (ii) induce the death of a cell to which they bind; (iii) inhibit the delamination of a cell to which they bind; (iv) inhibit the metastasis of a cell to which they bind; or (v) inhibit the vascularization of a tumor comprising a cell to which they bind.

Exemplary antibody inhibitors of TGF-β include e.g., Fresolimumab, antibody clone 1D11, and inhibitor LY3022859. Exemplary antibody inhibitors of activin include e.g., garetosmab, and the anti-activin antibodies disclosed in co-pending International Application No. PCT/US2021/065079, the disclosure of which is expressly incorporated by reference herein. In some embodiments, the anti-activin antibody comprises a heavy chain variable region/light chain variable region (HCVR/LCVR) sequence pair selected from the group consisting of SEQ ID NOs: 433/434, 435/436, 437/438, 439/440, 441/442, 443/444, 445/446, 447/448, 449/450, 451/452, 453/454, 455/456, 457/458, 459/460, 461/462, 463/464, 465/466, 467/468, 469/470, 471/472, 473/474, 475/476, 477/478, 479/480, 481/482, 483/484, 485/486, 487/488, 489/490, 491/492, 493/494, 495/496, 497/498, 499/500, and 501/502. In some embodiments, the anti-activin antibody comprises the six complementarity determining regions (CDRs) of a HCVR/LCVR sequence pair selected from the group consisting of SEQ ID NOs: 433/434, 435/436, 437/438, 439/440, 441/442, 443/444, 445/446, 447/448, 449/450, 451/452, 453/454, 455/456, 457/458, 459/460, 461/462, 463/464, 465/466, 467/468, 469/470, 471/472, 473/474, 475/476, 477/478, 479/480, 481/482, 483/484, 485/486, 487/488, 489/490, 491/492, 493/494, 495/496, 497/498, 499/500, and 501/502.

In some embodiments, the Activin inhibitor inhibits both Activin A and Activin B, where the Activin inhibitor is an anti-activin antibody comprising a HCVR/LCVR sequence pair selected from the group consisting of SEQ ID NOs: 471/472, 473/474, 475/476, 477/478, 479/480, 481/482, 495/496, 497/498, and 501/502; or is an anti-activin antibody comprising the six CDRs of a HVCR/LCVR sequence pair selected from the group consisting of SEQ ID NOs: 471/472, 473/474, 475/476, 477/478, 479/480, 481/482, 495/496, 497/498, and 501/502. Without being bound by theory, anti-activin antibodies capable of targeting both Activin A and Activin B may reduce the risk of tumor escape mechanisms and improve cancer treatment, particularly in cancers where both Activin A and Activin B are upregulated.

Exemplary Antibody inhibitors of Activin and TGF-β are also listed in Table 3 and Table 4.

### Ligand Trap

In some embodiments a “TGF-β inhibitor” or “activin inhibitor” is a ligand trap. Ligand traps are recombinant molecules which contain the ligand binding domains of the α-chain and the β-chain of a heterodimeric cytokine receptor fused to the constant parts of a human immunoglobulin G molecule. Ligand traps are artificial binding molecules which bind their corresponding cytokine with high specificity and with a higher affinity than a homodimeric fusion protein containing only two α-chains of the cytokine receptor (see e.g., (2005) Ligand Traps. In: Vohr H W. (eds) Encyclopedic Reference of Immunotoxicology. Springer, Berlin, Heidelberg. Sotatercept and luspatercept are ligand trap agents for activin II receptors A and B, respectively (see e.g., Curr Hematol Malig Rep. 2019 August; 14(4):346-351). Exemplary ligand trap inhibitors of Activin and TGF-β are also listed in Table 3 and Table 4.

### Immune Checkpoint Inhibitors

In another aspect, the disclosure provides compositions and method for improving the infiltration of immune-cells into solid cancers, such that treatment with anti-activin and TGF-β therapies in combination, or serially, with immune-checkpoint inhibitors leads to improved objective response rate and overall survival. Immune checkpoint inhibition, via blocking the inhibitory activity of PD-1 or CTLA-4, results in a reawakening of the immune system and tumor responses in a subset of patients in select indications. The majority of patients, however, do not respond, and of those that do respond, responses may be sub-optimal or short-lived. There have been a number of studies attempting to assess the mechanisms of resistance to checkpoint inhibition, and TGFb signaling is commonly implicated as a mechanism of therapeutic resistance. As disclosed in Example 2 herein, activin blockade through garetosmab, in combination with TGF-β inhibition utilizing antibody 1D11, improves objective response rate, tumor regression, and overall survival, in response to treatment PD1 inhibition with RMP1-14, by comparison to treatment with either garetosmab and RMP1-14, 1D11 and RMP1-14, or garetosmab and 1D11, or any agent alone. Thus, administration of a composition comprising activin, and TGF-β inhibitors in combination with an immune-checkpoint inhibitor improves response rates and survival in patients with solid tumors.

### Chimeric Antigen Receptor (CAR) T Cells

In another aspect, the disclosure provides compositions and methods for improving the function of CAR-T cell therapies by combining with an anti-Activin antibody as a monotherapy or in combination with an anti-TGF-β antibody. TGF-β signaling has been shown functionally to activate SMAD2/3 phosphorylation and impairing the proliferation, cytokine production, and cytotoxicity of CAR T cells (see Stuber et al. in 2020 Journal for ImmunoTherapy of Cancer (2020) 8.e000676). Blocking of TGF-β signaling to improve CAR-T function via CRISPR-mediated deletion of the TBG-β receptor is currently being explored as a strategy to combat this resistance mechanism (see Tang et al. JCI Insight (2020) 5.4). Despite these efforts, the addition of an activin antagonist may be necessary to maintain desired function.

### Combination Therapies

In one aspect, the disclosure provides compositions and methods for treating cancer with a combination of an agent that binds and blocks the ability of TGF-β to bind to TGFBR complexes and an agent that binds and blocks the ability of activin to bind to TGFBR/activin receptor complexes Compositions comprising combination therapies may include any of the agents listed in Table 1, together with an agent that binds and blocks the ability of activin to bind to activin Receptor Complexes, that may include any of the agents listed in Table 2. In some embodiments, such agents are systemically administrated to the patient at therapeutically effective doses. In some embodiments, the dose of either agents given as a combination may be lower than that of the agent given individually—given the combination treatment demonstrates additive behavior in vitro at saturating concentrations. In other embodiments, the dose of activin inhibitor may be higher than that of the TGF-β inhibitor because activin is more selectively localized to the tumor than TGF-β and has less reported toxicities in the clinic. In some embodiments, the composition for use in combination therapy further comprises an immune checkpoint inhibitor.

### Pharmaceutical Formulations

The present disclosure also provides “pharmaceutically acceptable” or “physiologically acceptable” formulations comprising at least one TGF-β inhibitor and at least one activin inhibitor. Such formulations can be administered in vivo to a subject in order to practice treatment methods.

As used herein, the terms “pharmaceutically acceptable” and “physiologically acceptable” refer to carriers, diluents, excipients and the like that can be administered to a subject, preferably without producing excessive adverse side-effects (e.g., nausea, abdominal pain, headaches, etc.). Such preparations for administration include sterile aqueous or non-aqueous solutions, suspensions, and emulsions.

Pharmaceutical formulations can be made from carriers, diluents, excipients, solvents, dispersion media, coatings, antibacterial and antifungal agents, isotonic and absorption delaying agents, and the like, compatible with administration to a subject. Such formulations can be contained in a tablet (coated or uncoated), capsule (hard or soft), microbead, emulsion, powder, granule, crystal, suspension, syrup or elixir. Supplementary active compounds and preservatives, among other additives, may also be present, for example, antimicrobials, anti-oxidants, chelating agents, and inert gases and the like.

Excipients can include a salt, an isotonic agent, a serum protein, a buffer or other pH-controlling agent, an anti-oxidant, a thickener, an uncharged polymer, a preservative or a cryoprotectant. Excipients used in compositions of the disclosure may further include an isotonic agent and a buffer or other pH-controlling agent. These excipients may be added for the attainment of preferred ranges of pH (about 6.0-8.0) and osmolarity (about 50-400 mmol/L). Examples of suitable buffers are acetate, borate, carbonate, citrate, phosphate and sulfonated organic molecule buffer. Such buffers may be present in a composition in concentrations from 0.01 to 1.0% (w/v). An isotonic agent may be selected from any of those known in the art, e.g. mannitol, dextrose, glucose and sodium chloride, or other electrolytes. Preferably, the isotonic agent is glucose or sodium chloride. The isotonic agents may be used in amounts that impart to the composition the same or a similar osmotic pressure as that of the biological environment into which it is introduced. The concentration of isotonic agent in the composition will depend upon the nature of the particular isotonic agent used and may range from about 0.1 to 10%. When glucose is used, it is preferably used in a concentration of from 1 to 5% w/v, more particularly 5% w/v. When the isotonic agent is sodium chloride, it is preferably employed in amounts of up to 1% w/v, in particular 0.9% w/v. The compositions of the disclosure may further contain a preservative. Examples preservatives are polyhexamethylene-biguanidine, benzalkonium chloride, stabilized oxychloro complexes (such as those known as Purite®), phenylmercuric acetate, chlorobutanol, sorbic acid, chlorhexidine, benzyl alcohol, parabens, and thimerosal. Typically, such preservatives are present at concentrations from about 0.001 to 1.0%. Furthermore, the compositions of the disclosure may also contain a cryopreservative agent. Exemplary cryopreservatives are glucose, sucrose, mannitol, lactose, trehalose, sorbitol, colloidal silicon dioxide, dextran of molecular weight preferable below 100,000 g/mol, glycerol, and polyethylene glycols of molecular weights below 100,000 g/mol or mixtures thereof. Most preferred are glucose, trehalose and polyethylene glycol. Typically, such cryopreservatives are present at concentrations from about 0.01 to 10%.

A pharmaceutical formulation can be formulated to be compatible with its intended route of administration. For example, for oral administration, a composition can be incorporated with excipients and used in the form of tablets, troches, capsules, e.g., gelatin capsules, or coatings, e.g., enteric coatings (Eudragit® or Sureteric®). Pharmaceutically compatible binding agents, and/or adjuvant materials can be included in oral formulations. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or other stearates; a glidant such as colloidal silicon dioxide; a sweetening agent such as sucrose or saccharin; or a flavoring agent such as peppermint, methyl salicylate, or flavoring.

Formulations can also include carriers to protect the composition against rapid degradation or elimination from the body, such as a controlled release formulation, including implants and microencapsulated delivery systems. For example, a time delay material such as glyceryl monostearate or glyceryl stearate alone, or in combination with a wax, may be employed.

Suppositories and other rectally administrable formulations (e.g., those administrable by enema) are also contemplated. Further regarding rectal delivery, see, for example, Song et al., Mucosal drug delivery: membranes, methodologies, and applications, Crit. Rev. Ther. Drug. Carrier Syst., 21:195-256, 2004; Wearley, Recent progress in protein and peptide delivery by noninvasive routes, Crit. Rev. Ther. Drug. Carrier Syst., 8:331-394, 1991.

Additional pharmaceutical formulations appropriate for administration are known in the art and are applicable in the methods and compositions of the disclosure (see, e.g., Remington's Pharmaceutical Sciences (1990) 18th ed., Mack Publishing Co., Easton, Pa.; The Merck Index (1996) 12th ed., Merck Publishing Group, Whitehouse, N.J.; and Pharmaceutical Principles of Solid Dosage Forms, Technonic Publishing Co., Inc., Lancaster, Pa., (1993)).

### Administration

In one embodiment, the composition comprising at least one TGF-β inhibitor and the at least one activin inhibitor is administered systemically. However, any of a number of administration routes are possible and the choice of a particular route will in part depend on the target tissue. Syringes, endoscopes, cannulas, intubation tubes, catheters and other articles may be used for administration.

The doses or “effective amount” for treating a subject are preferably sufficient to ameliorate one, several or all of the symptoms of the condition, to a measurable or detectable extent, although preventing or inhibiting a progression or worsening of the disorder or condition, or a symptom, is a satisfactory outcome. Thus, in the case of a condition or disorder treatable by administering at least one TGF-β inhibitor and the at least one activin inhibitor to a target tissue/tumor, the amount of at least one TGF-β inhibitor and the at least one activin inhibitor used to ameliorate a condition treatable by a method of the disclosure will depend on the condition and the desired outcome and can be readily ascertained by the skilled artisan. Appropriate amounts will depend upon the condition treated, the therapeutic effect desired, as well as the individual subject (e.g., the bioavailability within the subject, gender, age, etc.). The effective amount can be ascertained by measuring relevant physiological effects.

Veterinary applications are also contemplated by the present disclosure. Accordingly, in one embodiment, the disclosure provides methods of treating non-human mammals, which involve administering at least one TGF-β inhibitor and the at least one activin inhibitor if the disclosure to a non-human mammal in need of treatment.

### Parenteral Administration

The compounds of the disclosure may be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors and infusion techniques.

Parenteral formulations are typically aqueous solutions which may contain excipients such as salts, carbohydrates and buffering agents, but, for some applications, they may be more suitably formulated as a sterile non-aqueous solution or as a dried form to be used in conjunction with a suitable vehicle such as sterile, pyrogen-free water.

The preparation of parenteral formulations under sterile conditions, for example, by lyophilisation, may readily be accomplished using standard pharmaceutical techniques well known to those skilled in the art.

The solubility of compounds used in the preparation of parenteral solutions may be increased by the use of appropriate formulation techniques, such as the incorporation of solubility-enhancing agents.

Formulations for parenteral administration may be formulated to be immediate and/or modified release. Modified release formulations include delayed-, sustained-, pulsed-, controlled-, targeted and programmed release. Thus compounds of the disclosure may be formulated as a solid, semi-solid, or thixotropic liquid for administration as an implanted depot providing modified release of the active compound.

### Oral Administration

The subject compositions may be administered orally. Oral administration may involve swallowing, so that the compound enters the gastrointestinal tract. Compositions of the disclosure may also be administered directly to the gastrointestinal tract.

Formulations suitable for oral administration include solid formulations such as tablets, capsules, coated capsules containing particulates or coated particulates, liquids, or powders, lozenges (including liquid-filled), chews, multi- and nano-particulates, gels, films, ovules, and sprays.

Liquid formulations include suspensions, solutions, syrups and elixirs. Liquid formulations may be prepared by the reconstitution of a solid.

Tablet dosage forms generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate. Generally, the disintegrant will comprise from 1 weight % to 25 weight %, preferably from 5 weight % to 20 weight % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinised starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate.

Tablets may also optionally comprise surface active agents, such as sodium lauryl sulfate and polysorbate 80, and glidants such as silicon dioxide and talc. When present, surface active agents may comprise from 0.2 weight % to 5 weight % of the tablet, and glidants may comprise from 0.2 weight % to 1 weight % of the tablet.

Tablets also generally contain lubricants such as magnesium stearate, calcium stearate, zinc stearate, sodium stearyl fumarate, and mixtures of magnesium stearate with sodium lauryl sulphate. Lubricants generally comprise from 0.25 weight % to 10 weight %, preferably from 0.5 weight % to 3 weight % of the tablet.

Other possible ingredients include anti-oxidants, colorants, flavoring agents, preservatives and taste-masking agents.

Tablet blends may be compressed directly or by roller to form tablets. Tablet blends or portions of blends may alternatively be wet-, dry-, or melt-granulated, melt congealed, or extruded before tabletting. The final formulation may comprise one or more layers and may be coated or uncoated; it may even be encapsulated.

The formulation of tablets is discussed in Pharmaceutical Dosage Forms: Tablets, Vol. 1, by H. Lieberman and L. Lachman (Marcel Dekker, New York, 1980).

Consumable oral films for human or veterinary use are typically pliable water-soluble or water-swellable thin film dosage forms which may be rapidly dissolving or mucoadhesive and typically comprise a film-forming polymer, a binder, a solvent, a humectant, a plasticizer, a stabilizer or emulsifier, a viscosity-modifying agent and a solvent. Some components of the formulation may perform more than one function.

Also included in the disclosure are multiparticulate beads comprising a composition of the disclosure.

Other possible ingredients include anti-oxidants, colorants, flavorings and flavor enhancers, preservatives, salivary stimulating agents, cooling agents, co-solvents (including oils), emollients, bulking agents, anti-foaming agents, surfactants and taste-masking agents.

Films in accordance with the disclosure are typically prepared by evaporative drying of thin aqueous films coated onto a peelable backing support or paper. This may be done in a drying oven or tunnel, typically a combined coater dryer, or by freeze-drying or vacuuming.

Solid formulations for oral administration may be formulated to be immediate and/or modified release. Modified release formulations include delayed-, sustained-, pulsed-, controlled-, targeted and programmed release.

Other suitable release technologies such as high energy dispersions and osmotic and coated particles are known.

### Topical Administration

The compounds of the disclosure may also be administered topically to the skin or mucosa, that is, dermally or transdermally. Typical formulations for this purpose include gels, hydrogels, lotions, solutions, creams, ointments, dusting powders, dressings, foams, films, skin patches, wafers, implants, sponges, fibres, bandages and microemulsions.

Other means of topical administration include delivery by electroporation, iontophoresis, phonophoresis, sonophoresis and microneedle or needle-free (e.g. Powderject™, Bioject™, etc.) injection.

Formulations for topical administration may be formulated to be immediate and/or modified release. Modified release formulations include delayed-, sustained-, pulsed-, controlled-, targeted and programmed release.

### Inhaled/Intranasal Administration

The compounds of the disclosure can also be administered intranasally or by inhalation, typically in the form of a dry powder (either alone, as a mixture, for example, in a dry blend with lactose, or as a mixed component particle) from a dry powder inhaler or as an aerosol spray from a pressurized container, pump, spray, atomiser, or nebuliser, with or without the use of a suitable propellant.

Capsules, blisters and cartridges for use in an inhaler or insufflator may be formulated to contain a powder mix of the compound of the disclosure, a suitable powder base such as lactose or starch and a performance modifier such as I-leucine, mannitol, or magnesium stearate.

Formulations for inhaled/intranasal administration may be formulated to be immediate and/or modified release. Modified release formulations include delayed-, sustained-, pulsed-, controlled-, targeted and programmed release.

### Rectal/Intravaginal Administration

The compounds of the disclosure may be administered rectally or vaginally, for example, in the form of a suppository, pessary, or enema. Cocoa butter is a traditional suppository base, but various alternatives may be used as appropriate.

Formulations for rectal/vaginal administration may be formulated to be immediate and/or modified release. Modified release formulations include delayed-, sustained-, pulsed-, controlled-, targeted and programmed release.

### Ocular/Aural Administration

The compounds of the disclosure may also be administered directly to the eye or ear, typically in the form of drops. Other formulations suitable for ocular and aural administration include ointments, biodegradable (e.g. absorbable gel sponges, collagen) and non-biodegradable (e.g. silicone) implants, wafers, lenses and particulate systems. Formulations may also be delivered by iontophoresis.

Formulations for ocular/aural administration may be formulated to be immediate and/or modified release. Modified release formulations include delayed-, sustained-, pulsed-, controlled-, targeted, or programmed release.

### Methods of Use

In one embodiment, the compositions of the disclosure comprising at least one TGF-β inhibitor and at least one activin inhibitor may be used for therapeutic treatment. Such compositions are sometimes referred to herein as therapeutic compositions.

Therapeutic proteins of the disclosure e.g., protein based TGF-β inhibitors and protein based activin inhibitors, as discussed below, are produced utilizing therapeutic nucleic acids.

Therapeutic proteins contemplated for use as disclosed herein, typically have the ability to inhibit TGF-β or to inhibit activin and find use in the treatment of solid tumors enriched for cancer fibroblasts or for the treatment of fibrotic diseases. The following description of therapeutic protein activities, and indications treatable with therapeutic proteins disclosed herein, is exemplary and not intended to be exhaustive.

### EXAMPLES

Example 1: Combination treatment with TGF-β inhibitors and activin inhibitors blocks the ability of cancer cells to transform fibroblasts into CAFs in vitro

Fibroblasts may be transformed into cancer-associated fibroblasts (CAFs) through contact with a single exogenous ligand, such as transforming growth factor-(TGF-β). Thus, it has been hypothesized that it is certain cytokines secreted from cancer cells that promote conversion of resident fibroblasts into CAFs. However, in human tumors it is likely that cancer cells directly and indirectly induce CAFs through a variety of mechanisms. To test this hypothesis, and identify novel targets involved in CAF transformation, we examined the expression of CAF markers in co-cultures of fibroblasts and cancer cells versus fibroblast monocultures in which a single agent (e.g., a cytokine), is added to induce transformation of fibroblasts into a CAF-like state.

First, we treated normal human fibroblasts with a panel of cytokines to induce activation, and then assessed CAF marker expression by immunofluorescence. Fibroblast monocultures were serum-starved for 24 hours then stimulated with TGF-β, TNF alpha, PDGFbb, acidic FGF or other cytokines for 48 hours. Cells were then fixed, permeabilized and stained with a panel of immunofluorescent CAF markers and imaged by high-content microscopy. Of the tested cytokines/ligands, TGF-β increased expression in the greatest number of CAF markers compared to untreated fibroblasts (FIG. 1). In particular, stimulation with TGF-β increased expression of alpha-smooth muscle actin (αSMA), podoplanin (PDPN) and CD248 in the fibroblast cells. Incubation with TNFα increased PDPN expression, but not CD248 expression, consistent with the idea that PDPN and CD248 mark different fibroblast states. Notably, none of the cytokines tested significantly upregulated fibroblast activation protein (FAP), a key CAF marker. This suggests that monoculture fibroblasts can be activated, but do not transform into true CAFs with single cytokine stimulations.

In parallel to these cytokine stimulation tests, the same fibroblasts were co-cultured with two colorectal cancer (CRC) cell lines. In contrast to individual cytokine treatments, co-culture of fibroblasts with CRC cells resulted in a pronounced upregulation of FAP (FIG. 1). In fibroblast cells co-cultured with HCT116 cells, up-regulation of FAP occurred alongside a large increase in the variability of PDGFR-B and CD248 expression in single-cells. In fibroblast cells co-cultured with HT-29 cells, we observed a considerable up-regulation of all tested CAF-associated markers. Together these results showed that individual cytokines are able to induce the expression of key subsets of CAF-associated markers, but co-cultures of fibroblasts with cancer cells result in much broader CAF induction.

We then tested the relative potency of a panel of TGF-β pathway inhibitors in monoculture fibroblasts stimulated with TGF-β compared to co-culture with CRC cells that produce their own, similar levels of TGF-β. Specifically, we profiled five different small molecule inhibitors of TGF-β receptors [LY364947, LY2157299, SB-505124, SB431542 and LY2109761]; and SIS3, a drug that is proposed to disrupt the interface between SMAD3 protein and the co-SMAD, SMAD4 (Jinnin et al. 2006).

In brief, monocultures of BJ or CCD18-Co fibroblasts were stimulated with TGF-β for 48 hours in the presence of serial dilutions of each TGF-β inhibitor and fibroblast activation was assessed by immunofluorescence using αSMA and CD248. The probability of fibroblast activation was assessed by a machine learning model, Multiple Instance Learning (MIL) trained on unstimulated fibroblasts (no drug, no TGF-β) as a negative control, and TGF-β stimulated fibroblasts (with vehicle control) as positive control. For co-culture comparisons, the same fibroblasts were grown in the presence of either HCT 116 cells or HT-29 cells and treated with the same serial dilutions of small molecule TGF-β. For these co-cultures, the addition of exogenous TGF-β is not required as the cancer cells secrete sufficient TGF-β for maximal fibroblast activation. Indeed, we have previously shown that adding in exogenous TGF-β does not impact the effectiveness of TGF SMIs in coculture. The results of these experiments showed that all TGF-β inhibitors showed dramatically higher IC50 values in co-culture relative to mono-cultured fibroblasts. This strongly suggests that co-cultures are more resistant to TGF-β inhibition. This observation was confirmed by CellProfiler analysis where individual fibroblasts were segmented and αSMA and stress-fiber intensity was quantified (FIG. 2A). Notably, in co-culture with increasing doses of small molecule inhibitors, αSMA levels were only modestly reduced, while CD248 levels increased, and ultimately cell death was seen if higher concentrations were used (conditions with >5% cell death were excluded from analysis). Interestingly, the only small molecule inhibitor that was able to reduce αSMA levels in coculture by more than 50% of the control was SIS3.

In general, we observed that the more selective the TGFBR inhibitor, the greater the rightward shift (towards resistance) for the co-culture IC50 values. For example, LY2157299 (Galunisertib) is highly selective for ALK5 (TGFBR1) with an IC50 in the mid-nanomolar range. While LY2157299 was effective in monoculture, we observed very little inhibition of αSMA in co-culture even up to 100 μM. The other TGFBR SMIs tested have varying selectivity and rightward shifts of IC50 tended to be higher for more selective SMIs and SIS3, an inhibitor that hits a target downstream of TGFBRs (the least selective) shows the best response. Activin receptor (ACVR), like TGFBR, acts upstream of SMAD3. Accordingly, activin signaling as well as TGF-β signaling are blocked by SIS3. Based on these observations, we hypothesized that activin signaling through activin receptors and SMAD3 are a mechanism for co-culture resistance to TGF-β inhibition.

Small molecule inhibitors are much less selective for their respective targets than monoclonal antibodies (mAbs) that bind through highly selective hypervariable domains or ligand traps that bind through large protein interfaces that have naturally evolved. We took an mAb inhibitor of TGFBR2, LY3022859, and assessed the performance of this inhibitor in co-culture alongside SIS3 and LY364847, (FIG. 2 B). We found that even at very high doses for a mAb [>1.3 uM or 200 μg/mL], CAF phenotypes in co-culture remained unchanged following the addition of LY3022859. In contrast, both LY364947 and SIS3 were active at identical doses to those used in the previous test. Again, exogenous TGF-β was dispensable for this effect. Together, this demonstrates that co-cultures are resistant to TGF-β inhibition.

To test whether Activin signaling could induce CAF activation similarly to TGF-β we added serial dilutions (3-fold) of recombinant Activin A (500 ng/ul max concentration) and Activin B (150 ng/ul max concentration) to unstimulated fibroblasts (BJ, CCD18-Co) to generate dose response curves for Activin A and B. A serial 3-fold dilution of TGF-β was also tested as a positive control (30 ng/ul max concentration). A number of high-content markers of CAF activation we used as endpoints. Namely, cells were probed for SMAD2/3 translocation to the nucleus and phospoCREB levels at 1 hr, as a marker of direct ACVR/TGFBR stimulation. ASMA and Endosialin levels were then probed at 48 hrs to quantify CAF induction levels following Activin and TGF-β stimulation. At the highest dose, at 1 hr we saw SMAD 2/3 nuclear translocation alongside phospho-CREB up-regulation, indicating receptor engagement and activity in both Activin and TGF-β treated conditions (FIG. 3A). At 48 hours strong induction of ASMA and down-regulation of Endosialin was seen similar to TGF-β treatment as well (FIG. 3A), Quantitatively, an ASMA score based on the intensity and number of ASMA filaments detected showed no difference between Activin A and B treatment vs. TGF-β treatment at the highest dose (FIG. 3B). Moreover, analysis of the dose response curves using a machine learning approach (composite score learnt from high-content images), showed that both Activin A and Activin B could reach saturating doses in terms of fibroblast induction into a CAF like state (FIG. 3C). Of note however, EC50 values were lower for Activin A (10-30 ng/ul; depending on condition tested) and Activin B (4-9 ng/ul) vs. TGF-β (0.05/0.4 ng/ul), indicating that mature/free TGF-β is active at lower concentrations than Activin. Taken together this experiment demonstrated that both TGF-β and Activin can induce CAF like properties in normal fibroblastic cells, indicating they may cooperate in the Tumor Microenvironment to induce pro-tumor CAFs.

Based on these experimental results, and the observation that Activin A is uniquely upregulated in co-culture, unlike TGF-β (FIG. 4A), we hypothesized that Activin signaling is providing resistance to TGF-β inhibition, (potentially through an autocrine feedback loop). To test this, we treated cancer cell-fibroblast co-cultures with Activin neutralizing large molecules, either alone or in combination with TGF-β inhibitors. To ensure specificity, we dispensed with small molecules and employed Garetosmab, an Activin A ligand-specific mAb in clinical development, Follistatin a natural peptide that inhibits activin signaling and Fresolimumab, a pan-TGFβ ligand neutralizing mAb.

We established co-cultures of cancer cells and CAFs on substrates of varying stiffness, serum-starved in media with 0.5% FBS, and treated with each entity alone at 100 nM, or in combination, for 48 hours. Co-cultures were then fixed and stained with CAF activation markers, imaged, and analyzed. We observed that inhibiting Activin A and TGF-β ligands simultaneously leads to significant reduction in key markers of CAF activation across a range of co-culture models. This effect was observed on tissue culture plastic, as well as a tunable silicone substrate which provides physiologically relevant stiffness (FIG. 4B). Strikingly, CAF proliferation, CAF area and CAF marker expression such as αSMA and CD248 were significantly downregulated when both TGF-β and Activin ligands are neutralized in coculture (FIG. 4B). A similar effect was also seen when Follistatin was used in place of Garetosmab. The effect of combo treatment is highly similar to the effect of SIS3 on co-culture, supporting our hypothesis that activin A signaling contributes to CAF resistance to TGF-β inhibition. The combination of Activin A and TGF-β neutralizing IgGs was more effective than either monoculture treatment at blocking or reversing CAF activation in a range of cancer-fibroblast cultures in various culture conditions supporting our hypothesis that Activin mediates TGF-β resistance in vitro and providing an example of the value in treating TGF-β and Activin in combination in the clinic.

Example 2: Blockade of both TGF-β and activin in vivo potentiates the effect of immune-checkpoint inhibitors more than the combination of the immune checkpoint inhibitor with either activin or TGF-β alone.

The following Example illustrates that blocking TGF-β and activin in combination enhances the efficacy of immune-checkpoint inhibitors. We show in vivo inhibition of both TGF-β and activin A improves response rates to immune-checkpoint inhibition of PD1 in an EMT6 mouse model.

We performed a study to demonstrate the preclinical in vivo therapeutic efficacy of activin and TGF-β inhibition in combination with inhibition of the immune-checkpoint PD1 in the treatment of the subcutaneous syngeneic EMT6 breast cancer model in BALB/c mice. One mouse at a time was immobilized and the site of injection was disinfected with an alcohol swab. Each mouse was inoculated subcutaneously in the right flank region with EMT6 tumor cells (5×10⁵) in 0.1 mL of PBS solution for tumor development.

After tumor cell inoculation, the animals were checked daily for morbidity and mortality. During routine monitoring, the animals were also checked for any effects of tumor growth and treatments on behavior such as mobility, food and water consumption, body weight gain/loss (body weights were measured 3 times a week after randomization), eye/hair matting, and any other abnormalities. Mortality and observed clinical signs were recorded for individual animals in detail.

Tumor volumes were measured 3 times a week in two dimensions using a caliper, with the volume expressed in mm³ using the formula: “V=(L×W×W)/2, where V is tumor volume, L is tumor length (the longest tumor dimension) and W is tumor width (the longest tumor dimension perpendicular to L). Tumor volumes were recorded following inoculation, and mice were randomized to treatment groups when average tumor volume reached ˜120 mm³. The day of randomization is indicated as day 0. Dosing as well as tumor and body weight measurements was conducted in a Laminar Flow Cabinet. The body weights and tumor volumes were recorded using the StudyDirector™ software.

The anti-activin A antagonistic antibodies garetosmab and AMGA1, described in U.S. Pat. Nos. 9,718,881 and 8,309,082 respectively, were used to demonstrate the added benefit of activin neutralization on top of either anti-PD-1 or anti-TGF-β monotherapy or combination of PD-1 monotherapy and TGF-β inhibition. The mouse-cross-reactive anti-PD-1 antibody clone RMP1-14, and the anti-TGF-β antibody clone 1D11 are well characterized in the literature and were used as in vivo surrogates for their clinical comparator antibodies.

Study groups are indicated in Table 5 below. Dosing was performed on a mg/kg basis with intraperitoneal injections of antibodies diluted in sterile PBS. Doses were administered 3× week for 4 weeks.

Treatment of EMT6 tumor-bearing mice demonstrated enhanced effectiveness when with combination of PD-1 inhibition, TGB-β antagonism, and activin blocking were provided as a combination therapy. As shown in FIG. 5, anti-TGF-β and anti-activin monotherapy arms resulted in no appreciable change in tumor volume from vehicle control. Only anti-PD-1 antibody treatment had any effect as a mono-therapy, showing tumor regressions in 3 out of 10 mice.

Addition of TGF-β inhibition to the PD-1 blockade increased the number of tumor-free mice to 8/10, with regressions having occurred by day 28, within the treatment window for the study. The benefit of a triple combination was seen with the addition of the activin antagonist garetosmab which resulted in 8/10 tumor-free mice, but delayed outgrowth of tumors in the two mice who eventually progressed. The triple combination also resulted in increased survival relative to both the dual combination of anti-PD-1 and anti-TGF-β as well as the respective monotherapy arms (FIG. 6).

Overall, these data demonstrate that the combination of an anti-activin antagonist with PD-1 and TGF-β inhibition provides a more beneficial treatment in an EMT6 breast tumor model that is only minimally responsive to anti-PD-1 inhibition alone.

Example 3: Bioinformatics analysis demonstrates that Activin A and TGF-β are highly upregulated in the tumor microenvironment, correlate with immune-suppressive immune-cell gene signatures, are prognostic, and may engage in a positive feedback loop maintaining CAF state.

The following Example illustrates that dual inhibition of activin and TGF-β is likely needed to block CAF induction in humans. Specifically, An analysis of bulk RNA sequencing data from The Cancer Genome Atlas (TCGA) was performed. Much of this analysis involved the use of Gene signatures given in figure legends to define cell-types that were compiled from literature with internal validation of gene-signature accuracy based on localisation to scRNA data. Of note, the ecmCAF signature we define here is a CAF cell-type that is similar to the myCAF cell subtype defined in defined in Ela Elyada et al. in Cancer Discov. 9.8 (2019): 1102-23., and Cluster 8 as defined in Claudia X. Dominguez et al. in Cancer Discov. 10.2 (2020): 232-53.

An initial analysis of traditionally stromal rich cancers e.g breast, pancreatic, colorectal, head and neck, and lung cancer shows that these cancers have some of the highest levels of Activin (average of A and B) and TGF-β expression (Average of 3 isoforms) of all cancers (FIG. 7). Indeed comparison with the ecmCAF gene-signature that we define shows that both Activin and TGF-β mRNA expression levels are highly correlated with this gene signature across different cancer types (Spearman's Rank Correlations of 0.67 for Activin vs. CAF signature, P<0.001; 0.58 for TGF-β vs. CAF signature, P<0.001). Of note, an averaged signature of Activin and TGF-β demonstrates a higher level of correlation with a CAF gene signature than either cytokine alone (0.7, P<0.001) indicating that mRNA expression levels of both genes is a strong indicator of how stromally rich a given cancer is. In a more detailed analysis of Activin, TGF-β and ecmCAF signature up-regulation in cancer tissue vs. normal tissue in top ecmCAF enriched cancers (filtered for >20 adjacent normal tissue samples), we see that both Activin A and TGF-β are very highly up-regulated vs. adjacent normal tissue (P<0.001, in all cases), with the exception of lung-cancer. In lung cancer both of these proteins are in fact downregulated (P<0.001), despite an up-regulation of CAFs as measured by an ecmCAF signature (FIG. 8), of note we also see BMP3 down-regulation in all cancers analysed, BMP3 is a known inhibitor of Activin signaling further supporting the role of Activin in tumor-progression (Kokabu et al. 2012). Within pancreatic, colorectal and breast cancers mRNA expression levels also maintain a high level of correlation between samples from different patients (P<0.001 for correlation between all combinations of Activin, TGF-β and CAF signatures within Pancreatic, Colon and Breast cancers; FIG. 9). Overall, this indicates that both TGF-β and Activin are tightly co-regulated and significantly up-regulated in stromal rich cancers—indicating a functional role. The fact that, within the TCGA dataset, the average of a TGF-β and Activin signature correlates more strongly with a CAF signature than either gene alone also suggests that redundancy may exist between the two pathways, i.e. up-regulation of either protein may induce CAFs, highlighting a need to target both.

A key aspect of the invention is that by inhibiting Activin and TGF-β in combination we anticipate improving response rates to immune-checkpoint inhibitors. To highlight the value in this we in humans we also performed an analysis of how predictive TGF-β and Activin mRNA expression levels are of immune-cell infiltrate signatures in human cancers, by fitting linear regression models to the data. Specifically, an array consisting of mRNA expression levels per sample for all isoforms for TGF-β, Activin, TGF-β plus Activin, and the ecmCAF signature, was used to train a linear regression model to predict the averaged signature per-sample for a cytotoxic CD8+ T-cell, cytotoxic Natural Killer cell and immune-suppressive FOXP3+T-reg signatures (FIG. 10). Analysis was performed over all solid primary tumor samples stored in The Cancer Genome Atlas (TCGA). R2 values were captured from the model and used to compare the relative performance of different models. P values were derived from performing the same analysis, but replacing the different genes with a random set of gene expression levels, from the set of all possible genes, iterating on this 100 times and comparing, and comparing R2 values for TGF-β, Activin, TGF-β plus Activin, and the ecmCAF to this null distribution. This analysis shows that TGF-β levels alone are not predictive of CD8+ T-cells, NK-cells, and FOXP3+Treg signatures (R2=0.13, 0.11, 0.12 respectively, P>0.05 for all cases). Activin meanwhile is significantly predictive of a FOXP3+T-reg signature (R2=0.21, P<0.01), but not predictive of either a CD8+ T-cell or NK-cell signature, suggesting that it is likely a modulator of this cell-type vs. CD8+ T-cells or NK cells. Combined mrRNA expression levels of Activin and TGF-β meanwhile were more predictive of the FOXP3+ signature than either cytokine alone (R2=0.25 P<0.01), this further supports the notion that dual treatment of these two cytokines in humans will have a more significant effect on the immune state of the microenvironment than either alone. Of note, the CAF signature demonstrated a similar predictive ability to the combined TGF-β and Activin signatures and was highly predictive of FOXP3+T-regs (R2=0.31, P<0.001), indicating it is likely closely linked to combined mrRNA expression levels of combined TGF-β and Activin A.

To further reinforce the notion that Activin and TGF-β mRNA expression levels are predictive of the immune-state of the microenvironment, composite inflammatory and suppressive signatures were defined based on a number of pro-inflammatory and immune-suppressive biomarkers (FIG. 10). Linear regression models were trained to predict the mean inflammatory and suppressive signatures from the expression levels of Activin, TGF-β, Activin plus TGF-β, and genes in the ecmCAF signature. R2 values were derived as a measure of how well each model performed and P values were calculated from an equivalent null distribution. TGF-β alone was not significantly predictive of either an inflammatory signature (R2=0.16, 0.20 for inflammatory and suppressive respectively, P>0.05 in both cases). Activin was predictive of an immune-suppressive signature (R2=0.23, P<0.05), the combination was more of an immune-suppressive signature than either cytokine alone (R2=0.33 P<0.01), and the ecmCAF signature was yet further predictive of an immune-suppressive signature (R2=0.34, P<0.01). Taken together this analysis demonstrates that whilst, Activin alone is predictive of an immune-suppressive gene signature across all TCGA solid primary tumors, the combination of TGF-β levels and Activin levels are more predictive of an immune-suppressed state in all cases analysed. This again suggests that a level redundancy likely exists between Activin and TGF-β, and high-levels of one protein can make up for lower levels of the other and vice versa, and that both likely need to be targeted in combination in order to reduce CAF dependent immune-suppression in cancer.

To demonstrate the importance of both Activin and TGF-β in cancer progression, we examined the prognostic value of INHBA (Activin most expressed isoform), TGFB1 (TGF-β most expressed isoform), and an average of INHBA and TGB1, TCGA mRNA expression levels (FIG. 11). INHBA and TGFB1 were both highly prognostic in a number of cancers traditionally rich in stroma. Namely, Breast, Colorectal, Pancreatic, Head and Neck, Cervical, and Lung cancers, were tested. Only Lung cancer stood out as cases where lower INHBA or TGFB1 indicated a more favourable outcome. In most cases, a combined INHBA and TGFB1 signature was more predictive of outcome than either cytokine alone, further indicating that both of these proteins are important in tumor progression and/or staging.

As a final example of the value of combination treatment with both Activin and TGF-β inhibitors, we show that in single-cell RNA datasets from Colorectal Cancer (Lee et al. 2020) and Triple Negative Breast Cancer (Cox et al. 2020), a wide range of cells express TGF-β, whilst only CAFs (Stormal cells), and TAMs (Myeloid cells) express Activin (FIG. 12). In analysis of normal adjacent cells in the CRC dataset, we see also that whilst a level of TGF-β is expressed by normal cell-types, Activin is exclusively up-regulated in cancer. This links into our example showing that both Activin and TGF-β can induce CAFs from normal fibroblasts, and that whilst TGF-β is expressed by mono-cultures albeit at a lower level, Activin is exclusively up-regulated in co-culture. Collectively this suggests that TGF-β can initiate CAF activation, but that to reverse or fully block CAF induction both Activin and TGF-β need to be blocked in order to break the autocrine feedback loop in which CAFs secrete high-level of Activin that maintain the CAF state.

Certain modifications and improvements will occur to those skilled in the art upon a reading of the foregoing description. It should be understood that all such modifications and improvements have been deleted herein for the sake of conciseness and readability but are properly within the scope of the following claims.

TABLE 1
Protein sequences & accession ID for TGFbeta & Activin

| | | | UniProt | | |
|---|---|---|---|---|---|
| | | | Accession | SEQ ID | |
| Protein | Gene | Species | number | NO: | FASTA sequence |
| Transforming | TGFB1 | Homo | P01137 | SEQ ID | MPPSGLRLLLLLLPLLWLLV |
| growth factor | | sapiens | | NO: 1 | LTPGRPAAGLSTCKTIDMEL |
| beta-1 | | (Human) | | | VKRKRIEAIRGQILSKLRLA |
| proprotein | | | | | SPPSQGEVPPGPLPEAVLAL |
| | | | | | YNSTRDRVAGESAEPEPEPE |
| | | | | | ADYYAKEVTRVLMVETHNEI |
| | | | | | YDKFKQSTHSIYMFFNTSEL |
| | | | | | REAVPEPVLLSRAELRLLRL |
| | | | | | KLKVEQHVELYQKYSNNSWR |
| | | | | | YLSNRLLAPSDSPEWLSFDV |
| | | | | | TGVVRQWLSRGGEIEGFRLS |
| | | | | | AHCSCDSRDNTLQVDINGFT |
| | | | | | TGRRGDLATIHGMNRPFLLL |
| | | | | | MATPLERAQHLQSSRHRRAL |
| | | | | | DTNYCFSSTEKNCCVRQLYI |
| | | | | | DFRKDLGWKWIHEPKGYHAN |
| | | | | | FCLGPCPYIWSLDTQYSKVL |
| | | | | | ALYNQHNPGASAAPCCVPQA |
| | | | | | LEPLPIVYYVGRKPKVEQLS |
| | | | | | NMIVRSCKCS |
| Transforming | TGFB1 | Homo | P01137 | SEQ ID | ALDTNYCFSSTEKNCCVRQL |
| growth factor | | sapiens | [279-390] | NO: 2 | YIDFRKDLGWKWIHEPKGYH |
| beta-1 | | (Human) | | | ANFCLGPCPYIWSLDTQYSK |
| | | | | | VLALYNQHNPGASAAPCCVP |
| | | | | | QALEPLPIVYYVGRKPKVEQ |
| | | | | | LSNMIVRSCKCS |
| Transforming | TGFB2 | Homo | P61812 | SEQ ID | MHYCVLSAFLILHLVTVALS |
| growth factor | | sapiens | | NO: 3 | LSTCSTLDMDQFMRKRIEAI |
| beta-2 | | (Human) | | | RGQILSKLKLTSPPEDYPEP |
| proprotein, | | | | | EEVPPEVISIYNSTRDLLQE |
| Isoform 1 | | | | | KASRRAAACERERSDEEYYA |
| | | | | | KEVYKIDMPPFFPSENAIPP |
| | | | | | TFYRPYFRIVRFDVSAMEKN |
| | | | | | ASNLVKAEFRVFRLQNPKAR |
| | | | | | VPEQRIELYQILKSKDLTSP |
| | | | | | TQRYIDSKVVKTRAEGEWLS |
| | | | | | FDVTDAVHEWLHHKDRNLGF |
| | | | | | KISLHCPCCTFVPSNNYIIP |
| | | | | | NKSEELEARFAGIDGTSTYT |
| | | | | | SGDQKTIKSTRKKNSGKTPH |
| | | | | | LLLMLLPSYRLESQQTNRRK |
| | | | | | KRALDAAYCFRNVQDNCCLR |
| | | | | | PLYIDFKRDLGWKWIHEPKG |
| | | | | | YNANFCAGACPYLWSSDTQH |
| | | | | | SRVLSLYNTINPEASASPCC |
| | | | | | VSQDLEPLTILYYIGKTPKI |
| | | | | | EQLSNMIVKSCKCS |
| Transforming | TGFB2 | Homo | P61812-2 | SEQ ID | MHYCVLSAFLILHLVTVALS |
| growth factor | | sapiens | | NO: 4 | LSTCSTLDMDQFMRKRIEAI |
| beta-2 | | (Human) | | | RGQILSKLKLTSPPEDYPEP |
| proprotein, | | | | | EEVPPEVISIYNSTRDLLQE |
| Isoform 2 | | | | | KASRRAAACERERSDEEYYA |
| | | | | | KEVYKIDMPPFFPSETVCPV |
| | | | | | VTTPSGSVGSLCSRQSQVLC |
| | | | | | GYLDAIPPTFYRPYFRIVRF |
| | | | | | DVSAMEKNASNLVKAEFRVF |
| | | | | | RLQNPKARVPEQRIELYQIL |
| | | | | | KSKDLTSPTQRYIDSKVVKT |
| | | | | | RAEGEWLSFDVTDAVHEWLH |
| | | | | | HKDRNLGFKISLHCPCCTFV |
| | | | | | PSNNYIIPNKSEELEARFAG |
| | | | | | IDGTSTYTSGDQKTIKSTRK |
| | | | | | KNSGKTPHLLLMLLPSYRLE |
| | | | | | SQQTNRRKKRALDAAYCFRN |
| | | | | | VQDNCCLRPLYIDFKRDLGW |
| | | | | | KWIHEPKGYNANFCAGACPY |
| | | | | | LWSSDTQHSRVLSLYNTINP |
| | | | | | EASASPCCVSQDLEPLTILY |
| | | | | | YIGKTPKIEQLSNMIVKSCK |
| | | | | | CS |
| Transforming | TGFB2 | Homo | P61812 | SEQ ID | ALDAAYCFRNVQDNCCLRPL |
| growth factor | | sapiens | [303-414] | NO: 5 | YIDFKRDLGWKWIHEPKGYN |
| beta-2 | | (Human) | | | ANFCAGACPYLWSSDTQHSR |
| | | | | | VLSLYNTINPEASASPCCVS |
| | | | | | QDLEPLTILYYIGKTPKIEQ |
| | | | | | LSNMIVKSCKCS |
| Transforming | TGFB3 | Homo | P10600 | SEQ ID | MKMHLQRALVVLALLNFATV |
| growth factor | | sapiens | | NO: 6 | SLSLSTCTTLDFGHIKKKRV |
| beta-3 | | (Human) | | | EAIRGQILSKLRLTSPPEPT |
| proprotein, | | | | | VMTHVPYQVLALYNSTRELL |
| Isoform 1 | | | | | EEMHGEREEGCTQENTESEY |
| | | | | | YAKEIHKFDMIQGLAEHNEL |
| | | | | | AVCPKGITSKVFRFNVSSVE |
| | | | | | KNRTNLFRAEFRVLRVPNPS |
| | | | | | SKRNEQRIELFQILRPDEHI |
| | | | | | AKQRYIGGKNLPTRGTAEWL |
| | | | | | SFDVTDTVREWLLRRESNLG |
| | | | | | LEISIHCPCHTFQPNGDILE |
| | | | | | NIHEVMEIKFKGVDNEDDHG |
| | | | | | RGDLGRLKKQKDHHNPHLIL |
| | | | | | MMIPPHRLDNPGQGGQRKKR |
| | | | | | ALDTNYCFRNLEENCCVRPL |
| | | | | | YIDFRQDLGWKWVHEPKGYY |
| | | | | | ANFCSGPCPYLRSADTTHST |
| | | | | | VLGLYNTLNPEASASPCCVP |
| | | | | | QDLEPLTILYYVGRTPKVEQ |
| | | | | | LSNMVVKSCKCS |
| Transforming | TGFB3 | Homo | P10600-2 | SEQ ID | MKMHLQRALVVLALLNFATV |
| growth factor | | sapiens | | NO: 7 | SLSLSTCTTLDFGHIKKKRV |
| beta-3 | | (Human) | | | EAIRGQILSKLRLTSPPEPT |
| proprotein, | | | | | VMTHVPYQVLALYNSTRELL |
| Isoform 2 | | | | | EEMHGEREEGCTQENTESEY |
| | | | | | YAKEIHKFDMIQGLAEHNEL |
| | | | | | AVCPKGITSKVFRFNVSSVE |
| | | | | | KNRTNLFRAEFRVLRVPNPS |
| | | | | | SKRNEQRIELFQILRPDEHI |
| | | | | | AKQRYIGGKNLPTRGTAEWL |
| | | | | | SFDVTDTVREWLLRRESNLG |
| | | | | | LEISIHCPCHTFQPNGDILE |
| | | | | | NIHEVMEIKFKGVDNEDDHG |
| | | | | | RGDLGRLKKQKDHHNPHLIL |
| | | | | | MMIPPHRLDNPGQGGQRKKR |
| | | | | | ALDTNYCFR |
| Transforming | TGFB3 | Homo | P10600 | SEQ ID | ALDTNYCFRNLEENCCVRPL |
| growth factor | | sapiens | | NO: 8 | YIDFRQDLGWKWVHEPKGYY |
| beta-3 | | (Human) | | | ANFCSGPCPYLRSADTTHST |
| | | | | | VLGLYNTLNPEASASPCCVP |
| | | | | | QDLEPLTILYYVGRTPKVEQ |
| | | | | | LSNMVVKSCKCS |
| Inhibin beta | INHBA | Homo | P08476 | SEQ ID | MPLLWLRGFLLASCWIIVRS |
| A chain | | sapiens | | NO: 9 | SPTPGSEGHSAAPDCPSCAL |
| precursor | | (Human) | | | AALPKDVPNSQPEMVEAVKK |
| | | | | | HILNMLHLKKRPDVTQPVPK |
| | | | | | AALLNAIRKLHVGKVGENGY |
| | | | | | VEIEDDIGRRAEMNELMEQT |
| | | | | | SEIITFAESGTARKTLHFEI |
| | | | | | SKEGSDLSVVERAEVWLFLK |
| | | | | | VPKANRTRTKVTIRLFQQQK |
| | | | | | HPQGSLDTGEEAEEVGLKGE |
| | | | | | RSELLLSEKVVDARKSTWHV |
| | | | | | FPVSSSIQRLLDQGKSSLDV |
| | | | | | RIACEQCQESGASLVLLGKK |
| | | | | | KKKEEEGEGKKKGGGEGGAG |
| | | | | | ADEEKEQSHRPFLMLQARQS |
| | | | | | EDHPHRRRRRGLECDGKVNI |
| | | | | | CCKKQFFVSFKDIGWNDWII |
| | | | | | APSGYHANYCEGECPSHIAG |
| | | | | | TSGSSLSFHSTVINHYRMRG |
| | | | | | HSPFANLKSCCVPTKLRPMS |
| | | | | | MLYYDDGQNIIKKDIQNMIV |
| | | | | | EECGCS |
| Inhibin beta | INHBA | Homo | P08476 | SEQ ID | GLECDGKVNICCKKQFFVSF |
| A chain | | sapiens | [311-426] | NO: 10 | KDIGWNDWIIAPSGYHANYC |
| | | (Human) | | | EGECPSHIAGTSGSSLSFHS |
| | | | | | TVINHYRMRGHSPFANLKSC |
| | | | | | CVPTKLRPMSMLYYDDGQNI |
| | | | | | IKKDIQNMIVEECGCS |
| Inhibin beta | INHBB | Homo | P09529 | SEQ ID | MDGLPGRALGAACLLLLAAG |
| B chain | | sapiens | | NO: 11 | WLGPEAWGSPTPPPTPAAPP |
| precursor | | (Human) | | | PPPPPGSPGGSQDTCTSCGG |
| | | | | | FRRPEELGRVDGDFLEAVKR |
| | | | | | HILSRLQMRGRPNITHAVPK |
| | | | | | AAMVTALRKLHAGKVREDGR |
| | | | | | VEIPHLDGHASPGADGQERV |
| | | | | | SEIISFAETDGLASSRVRLY |
| | | | | | FFISNEGNQNLFVVQASLWL |
| | | | | | YLKLLPYVLEKGSRRKVRVK |
| | | | | | VYFQEQGHGDRWNMVEKRVD |
| | | | | | LKRSGWHTFPLTEAIQALFE |
| | | | | | RGERRLNLDVQCDSCQELAV |
| | | | | | VPVFVDPGEESHRPFVVVQA |
| | | | | | RLGDSRHRIRKRGLECDGRT |
| | | | | | NLCCRQQFFIDFRLIGWNDW |
| | | | | | IIAPTGYYGNYCEGSCPAYL |
| | | | | | AGVPGSASSFHTAVVNQYRM |
| | | | | | RGLNPGTVNSCCIPTKLSTM |
| | | | | | SMLYFDDEYNIVKRDVPNMI |
| | | | | | VEECGCA |
| Inhibin beta | INHBB | Homo | P09529 | SEQ ID | GLECDGRTNLCCRQQFFIDF |
| B chain | | sapiens | [293-407] | NO: 12 | RLIGWNDWIIAPTGYYGNYC |
| | | (Human) | | | EGSCPAYLAGVPGSASSFHT |
| | | | | | AVVNQYRMRGLNPGTVNSCC |
| | | | | | IPTKLSTMSMLYFDDEYNIV |
| | | | | | KRDVPNMIVEECGCA |

TABLE 2
Immune-targets that could be combined with TGF-β and
Activin Blockade

| Target | Function | Developed by | Phase of Development |
|---|---|---|---|
| LAG-3 | inhibitory | BMS, Merck, MacroGenics, | various phase 1 and 2 |
| | | BI, Novartis, GSK, | trials |
| | | Regeneron/Sanofi, Roche, | |
| | | Tesaro, EMD | |
| TIGIT | inhibitory | Genentech, EMD, BMS, | various phase 1, 2, |
| | | Arcus, BeiGene, OncoMed, | and 3 trials |
| | | Astellas/Potenza, Innovent, | |
| | | Compugen, iTeos | |
| TIM-3 | inhibitory | Novartis, Tesaro, Roche, | various phase 1 and 2 |
| | | BeiGene, Lilly, Symphogen, | trials |
| | | Agenus, Zymeworks | |
| OX40 | activating | MedImmune, Pfizer, GSK, | various phase 1 and 2 |
| | | BMS, Genentech, Kirin, | trials |
| | | Kymab/Sanofi, Agenus/ | |
| | | Incyte, Glenmark, | |
| | | Alligator, Innovent, F-star, | |
| | | Beigene, Inhibrx | |
| 4-1BB/ | activating | BMS, Pfizer/Morphosys, | various phase 1 and 2 |
| CD137 | | Inhibrx, Lyvgen Biopharma, | trials |
| | | Adagene, Merus, Alligator, | |
| | | BioNTech/Genmab, | |
| | | Crescendo, Agenus/Gilead, | |
| | | Compass, f-star, ABL Bio, | |
| | | Numab | |
| GITR | activating | 4-Antibody/Agenus/Incyte, | various phase 1 and 2 |
| | | GITR/Leap Therapeutics/ | trials |
| | | Tolerx, Merck, Amgen, | |
| | | MedImmune, BMS, | |
| | | Regeneron, EMD | |
| ICOS | activating | GSK, Jounce, Kymab/ | various phase 1, 2. |
| | | Sanofi, Xencor, BMS | and 3 trials |
| CD40 | activating | Apexigen, Novartis, | various phase 1, 2. |
| | | Genentech/Seattle Genetics, | and 3 trials |
| | | Amgen/Pfizer/Abgenix, BI, | |
| | | Astellas, Biogen/Nektar, | |
| | | Celldex, Xoma, Anelixis, | |
| | | Alligator/Janssen, BMS, | |
| | | MedImmune, Domantis, | |
| | | Abbvie, Sanofi/ImmuNext, | |
| | | BioNTech/Genmab, | |
| | | PsiOxus, Lyvgen, Euchre, | |
| | | Kiniksa | |

TABLE 3
Example Biological TGF-β inhibitors

| | | Primary | | Dates | |
|---|---|---|---|---|---|
| Modality | Company | Indication | Stage | Filed | Patent # |
| Isoform | BMS | Solid cancers, | Phase 2 | 2008- | WO201703763, |
| selective ligand | (Acquired | Gastric | | 2020 | WO2017037634, |
| trap | Forbius) | | | | US20200231652, |
| (AVID200) | | | | | WO2018158727, |
| | | | | | U.S. Pat. No. 8,574,548, |
| | | | | | WO2008113185 |
| TGF-β ligand | GSK/ | Cancer/IO | Phase 2/3 | 2015- | U.S. Pat. No. 9,676,863, |
| trap/PDL1 | Merck | Combination | | 209 | WO2015118175, |
| bispecific | | | | | US20150225483, |
| (bintrafusp | | | | | US20180118832, |
| alfa, M7824) | | | | | WO2018029367, |
| | | | | | WO2020041607 |
| Antibody | Novartis | Cancer | Phase 2 | 2012- | U.S. Pat. No. 8,569,462, |
| (NIS793, | | | | 2019 | US20120315282, |
| XOMA 089) | | | | | WO2012167143, |
| | | | | | U.S. Pat. No. 9,145,458, |
| | | | | | US20140023644, |
| | | | | | U.S. Pat. No. 9,714,285, |
| | | | | | US20150322146, |
| | | | | | U.S. Pat. No. 10/167,334, |
| | | | | | US20160289315, |
| | | | | | WO2016161410, |
| | | | | | US20190092846, |
| | | | | | WO2017141208, |
| | | | | | U.S. Pat. No. 10/358,486, |
| | | | | | US20180057578, |
| | | | | | U.S. Pat. No. 10/683,347, |
| | | | | | US20190315850 |
| Antibody | Sanofi | IPF, Kidney | Phase 2 | 2006- | U.S. Pat. No. 10/730,936, |
| (fresolimumab, | (Acquired | fibrosis, Cancer | | 2017 | U.S. Pat. No. 10/526,402, |
| GC1008, | Genzyme) | | | | US20180051075, |
| PET1073G12) | | | | | US20180044412, |
| | | | | | US20170342144, |
| | | | | | U.S. Pat. No. 9,783,604, |
| | | | | | WO2016141244, |
| | | | | | WO2016141245, |
| | | | | | WO2016138301, |
| | | | | | WO2014164709, |
| | | | | | U.S. Pat. No. 8,591,901, |
| | | | | | WO2012030394, |
| | | | | | US20110008364, |
| | | | | | WO2006089251 |
| Antibody | Eli Lilly | Diabetic Kidney | Phase 2 | 2006- | WO2006116002. |
| (LY2382770) | | Disease | (discontinued) | 2010 | U.S. Pat. No. 7,494,651, |
| | | | | | WO2007076391, |
| | | | | | U.S. Pat. No. 7,619,069, |
| | | | | | US20100040633, |
| | | | | | U.S. Pat. No. 8,128,927, |
| | | | | | US20100136021, |
| | | | | | U.S. Pat. No. 7,927,593 |
| Antibody | Sanofi | Cancer | Phase 1/2 | 2018- | U.S. Pat. No. 10/766,955, |
| (SAR439459) | | | | 2020 | US20180244763, |
| | | | | | WO2018134681, |
| | | | | | WO2020012383, |
| | | | | | US20200399358 |
| Antibody | Genzyme/CAT | Sclerodoma | Phase 1/2 | 2000- | WO2000066631, |
| (metelimumab, | | | (discont.) | 2002 | U.S. Pat. No. 7,151,169 |
| CAT-192) | | | | | |
| Antibody | Genzyme/CAT | Glaucoma | Preclin | 1997- | WO1997013844, |
| (lerdelimumab, | | | (discont.) | 2003 | U.S. Pat. No. 7,368,111, |
| CAT-152, | | | | | US20050049403 |
| Trabio, 6B1) | | | | | |
| Antibody | Merck | N/A | Preclin. | 2017- | U.S. Pat. No. 10/287,347, |
| (TLS-01) | (Acquired | | | 2020 | U.S. Pat. No. 10/017,567, |
| | Tilos) | | | | US20180030128, |
| | | | | | US20180009886, |
| | | | | | US20190144549, |
| | | | | | WO2019075090, |
| | | | | | US20200040068, |
| | | | | | US20200140530, |
| | | | | | WO2020076969 |
| Antibody | Ludwig | Cancer | Preclin. | 2013- | U.S. Pat. No. 9,518,112, |
| (13A1) | Institute for | | | 2019 | US20150132319, |
| | Cancer | | | | WO2013134365, |
| | Research | | | | U.S. Pat. No. 10/035,851, |
| | | | | | US20170137507, |
| | | | | | U.S. Pat. No. 10/947/303, |
| | | | | | US20190092847, |
| | | | | | WO2020095113 |
| Antibody | Scholar | Solid tumors | Phase 1 | 2017- | US20190071493, |
| (SRK-181) | Rock | | | 2020 | WO2017156500, |
| | | | | | US20190292254, |
| | | | | | WO2018013939, |
| | | | | | US20190085067, |
| | | | | | US20200079840, |
| | | | | | WO2020014460, |
| | | | | | WO2020014473, |
| | | | | | US20200131259 |
| Ligand trap | Acceleron | Cancer, Fibrotic | Preclin. | 2014- | U.S. Pat. No. 10/316,076, |
| (ACE-1332) | Pharmaceuticals | diseases | | 2020 | WO2015027082, |
| | | | | | US20200157182, |
| | | | | | US20190284257 |

TABLE 4
Example Biological Inhibitors

| | | Primary | | Dates | |
|---|---|---|---|---|---|
| Modality | Company | Indication | Stage | Filed | Patent # |
| Antibody | Regeneron | FOP | Phase 2 | 2014-2018 | US20190153085, |
| (garetosmab or | Pharmaceuticals | | | | US20180008672, |
| REGN2477) | | | | | U.S. Pat. No. 9,718,881 |
| ACVR2A Fc- | Acceleron | PAH | Phase 2 | 2014 | WO2016069234A1 |
| Fusion Protein | Pharmaceuticals | | | | |
| (sotatercept, | | | | | |
| ACE-011) | | | | | |
| ACVR2B Fc- | Atara | Cancer | Phase 1 | 2014 | US201462012104P |
| Fusion Protein | Biotherapeutics | Cachexia | | | |
| (STM434) | | | | | |
| Follistatin-288- | Acceleron | Charcot- | Phase 2, | 2009-2020 | US20150158923, |
| Fc (efmitermant | Pharmaceuticals | Marie- | discont. | | US20200360475, |
| alfa, ACE-083) | | Tooth | | | WO2009158025, |
| | | disease | | | U.S. Pat. No. 9,919,030 |
| Follistatin-315- | Takeda | DMD | Pre-clin. | 2014-2019 | WO2014116981A1, |
| Fc fusion | (Acquired | | | | U.S. Pat. No. 10/723,772, |
| protein | Shire) | | | | US20180305421 |
| Antibody (A1) | Amgen | Cancer | Pre-clin. | 2007-2019 | U.S. Pat. No. 8,309,082, |
| | | Cachexia | | | US20150086556, |
| | | | | | US20180371073, |
| | | | | | WO2008031061, |
| | | | | | US20130030159, |
| | | | | | US20200108085, |
| | | | | | US20170073408, |
| | | | | | U.S. Pat. No. 10,100,109, |
| | | | | | WO2014121221, |
| | | | | | US20140220033, |
| | | | | | US20150359850, |
| | | | | | U.S. Pat. No. 8,753,627, |
| | | | | | US20090234106 |

TABLE 5
Study groups in EMT6 study of tumor volume inhibition and survival

| | | | Dose | | |
|---|---|---|---|---|---|
| | | | level | | Dosing Frequency |
| Group | No. | Treatment | (mg/kg) | ROA | & Duration |
| 1 | 10 | Vehicle (PBS) | | i.p. | BIW × 3 weeks |
| 2 | 10 | Anti-PD-1 | 10 | i.p. | BIW × 3 weeks |
| 3 | 10 | AMGA1 | 10 | i.p. | BIW × 3 weeks |
| 4 | 10 | 1D11 | 5 | i.p. | BIW × 3 weeks |
| 5 | 10 | Anti-PD-1 | 10 | i.p | BIW × 3 weeks |
| | | AMGA1 | 10 | i.p | BIW × 3 weeks |
| 6 | 10 | Anti-PD-1 | 10 | i.p | BIW × 3 weeks |
| | | 1D11 | 5 | i.p | BIW × 3 weeks |
| 7 | 10 | AMGA1 | 10 | i.p | BIW × 3 weeks |
| | | 1D11 | 5 | i.p | BIW × 3 weeks |
| 8 | 10 | Anti-PD-1 | 10 | i.p | BIW × 3 weeks |
| | | AMGA1 | 10 | i.p | BIW × 3 weeks |
| | | 1D11 | 5 | i.p | BIW × 3 weeks |
| 9 | 10 | Anti-PD-1 | 10 | i.p | BIW × 3 weeks |
| | | Garo | 10 | i.p | BIW × 3 weeks |
| | | 1D11 | 5 | i.p | BIW × 3 weeks |

TABLE 6
Complementarity Determining Regions, Heavy Variable Region (IMGT)

| | Heavy Chain Variable Region CDRs | | |
|---|---|---|---|
| Name | CDR1 | CDR2 | CDR3 |
| AB448 | GLSLSNAKMG | VSTTGTT | ARAGYPDV |
| | (SEQ ID NO: 13) | (SEQ ID NO: 48) | (SEQ ID NO: 83) |
| AB465 | GASVSNARTG | INPSGRI | ARAGYPDV |
| | (SEQ ID NO: 14) | (SEQ ID NO: 49) | (SEQ ID NO: 84) |
| AB340 | GFTASRNY | ISGSGDYT | ARSPRGPRLLGYYYGMDV |
| | (SEQ ID NO: 15) | (SEQ ID NO: 50) | (SEQ ID NO: 85) |
| AB342 | GFDFGEHW | IKGDASDT | VKDLSKQWLGIDY |
| | (SEQ ID NO: 16) | (SEQ ID NO: 51) | (SEQ ID NO: 86) |
| AB344 | GYTFSNYG | ISKSGSSI | ARLPRLAWNYYYGMDV |
| | (SEQ ID NO: 17) | (SEQ ID NO: 52) | (SEQ ID NO: 87) |
| AB353 | GLSLSNAKMG | VSTTGTT | ARAGYPDV |
| | (SEQ ID NO: 18) | (SEQ ID NO: 53) | (SEQ ID NO: 88) |
| AB438 | GVSLSNARLG | GSVTGST | TTSYLVDTAMVTR |
| | (SEQ ID NO: 19) | (SEQ ID NO: 54) | (SEQ ID NO: 89) |
| AB366 | GYSLTDARIG | INPSGRT | ARAGYPDV |
| | (SEQ ID NO: 20) | (SEQ ID NO: 55) | (SEQ ID NO: 90) |
| AB341 | GYTFSNYG | ISKSGSSI | ARLPRLAWNYYYGMDV |
| | (SEQ ID NO: 21) | (SEQ ID NO: 56) | (SEQ ID NO: 91) |
| AB430 | GFTLSNYP | TTSGSSTI | ARGPDYYAPLRSYMDV |
| | (SEQ ID NO: 22) | (SEQ ID NO: 57) | (SEQ ID NO: 92) |
| AB349 | GVSLSNARLG | GSVTGST | TTSYLVDTAMVTR |
| | (SEQ ID NO: 23) | (SEQ ID NO: 58) | (SEQ ID NO: 93) |
| AB449 | GLSLSNAKMG | VSTTGTT | ARAGYPDV |
| | (SEQ ID NO: 24) | (SEQ ID NO: 59) | (SEQ ID NO: 94) |
| AB338 | GFTASRNY | ISGSGDYT | ARSPRGPRLLGYYYGMDV |
| | (SEQ ID NO: 25) | (SEQ ID NO: 60) | (SEQ ID NO: 95) |
| AB435 | GYSLTDARIG | INPSGRT | ARAGYPDV |
| | (SEQ ID NO: 26) | (SEQ ID NO: 61) | (SEQ ID NO: 96) |
| AB372 | GFSLSTSGAT | ITSSGR | TTTKIVGATLRDY |
| | (SEQ ID NO: 27) | (SEQ ID NO: 62) | (SEQ ID NO: 97) |
| AB439 | GYTYTGYR | IDPKSGRT | AREVKQWLVPGRGVYWFDP |
| | (SEQ ID NO: 28) | (SEQ ID NO: 63) | (SEQ ID NO: 98) |
| AB350 | GVSLSDARMG | VSTTGTT | ARAGYPDV |
| | (SEQ ID NO: 29) | (SEQ ID NO: 64) | (SEQ ID NO: 99) |
| AB363 | GFSLSTSGAT | ITSSGR | TTTKIVGATLRDY |
| | (SEQ ID NO: 30) | (SEQ ID NO: 65) | (SEQ ID NO: 100) |
| AB367 | GFTLSNPRMG | ISTSGRT | ARAGYPDV |
| | (SEQ ID NO: 31) | (SEQ ID NO: 66) | (SEQ ID NO: 101) |
| AB352 | GFPLSTSRMG | INPSGAT | ARAGYPDV |
| | (SEQ ID NO: 32) | (SEQ ID NO: 67) | (SEQ ID NO: 102) |
| AB357 | GYTFPSHY | MNPITGHT | ARDMRYDILPGRSYYYGMDV |
| | (SEQ ID NO: 33) | (SEQ ID NO: 68) | (SEQ ID NO: 103) |
| AB347 | GFPLSTSRMG | INPSGAT | ARAGYPDV |
| | (SEQ ID NO: 34) | (SEQ ID NO: 69) | (SEQ ID NO: 104) |
| AB346 | GFPLSTSRMG | INPSGAT | ARAGYPDV |
| | (SEQ ID NO: 35) | (SEQ ID NO: 70) | (SEQ ID NO: 105) |
| AB433 | GFPLSTSRMG | INPSGAT | ARAGYPDV |
| | (SEQ ID NO: 36) | (SEQ ID NO: 71) | (SEQ ID NO: 106) |
| AB432 | GVSLSNVRMG | VNPSGST | ARAGYPDV |
| | (SEQ ID NO: 37) | (SEQ ID NO: 72) | (SEQ ID NO: 107) |
| AB326 | GYTFTSSA | IYPGSGHT | AREGDIFVMDV |
| | (SEQ ID NO: 38) | (SEQ ID NO: 73) | (SEQ ID NO: 108) |
| AB428 | GFTSSNHW | IGPKGLGT | ASIRDYGDKKGGSWFDP |
| | (SEQ ID NO: 39) | (SEQ ID NO: 74) | (SEQ ID NO: 109) |
| AB426 | GFTFSDHH | ISPSGDFT | ARDVSPVYY |
| | (SEQ ID NO: 40) | (SEQ ID NO: 75) | (SEQ ID NO: 110) |
| AB345 | GFTSSGYW | IGTAGVT | ARGPHGPQQRLRFDP |
| | (SEQ ID NO: 41) | (SEQ ID NO: 76) | (SEQ ID NO: 111) |
| AB431 | GFTSSNHW | IGPKGLGT | ARDVRGWRALYYYYGMDV |
| | (SEQ ID NO: 42) | (SEQ ID NO: 77) | (SEQ ID NO: 112) |
| AB330 | GYPSPAT | MNPKSGYT | ATLHGSGLAFDI |
| | (SEQ ID NO: 43) | (SEQ ID NO: 78) | (SEQ ID NO: 113) |
| AB731 | GYTFTSYW | INPSNGGT | SITTVVAYWYFDV |
| | (SEQ ID NO: 44) | (SEQ ID NO: 79) | (SEQ ID NO: 114) |
| AB843 | GDTSSHSH | INPRSGAT | ARDRGGRTNGVGAFDI |
| | (SEQ ID NO: 45) | (SEQ ID NO: 80) | (SEQ ID NO: 115) |
| AB852 | GFSLSDTRWG | INDRGTT | AHTLHTREFVGMDV |
| | (SEQ ID NO: 46) | (SEQ ID NO: 81) | (SEQ ID NO: 116) |
| AB854 | GYTFPSHY | MNPITGHT | ARDMRYDILPGRSYYYGMDV |
| | (SEQ ID NO: 47) | (SEQ ID NO: 82) | (SEQ ID NO: 117) |

TABLE 7
Complementarity Determining Regions, Light Variable Region (IMGT)

| | Light Chain Variable Region CDRs | | |
|---|---|---|---|
| Name | CDR1 | CDR2 | CDR3 |
| AB448 | QNIGAA | GAS | QQYTAYPLT |
| | (SEQ ID NO: 118) | (SEQ ID NO: 153) | (SEQ ID NO: 188) |
| AB465 | QTVSSH | DVS | QQYFTLPT |
| | (SEQ ID NO: 119) | (SEQ ID NO: 154) | (SEQ ID NO: 189) |
| AB340 | RSLVHGSGHNY | GAS | MQGTQAPRT |
| | (SEQ ID NO: 120) | (SEQ ID NO: 155) | (SEQ ID NO: 190) |
| AB342 | QSLLHSNEKNY | DAS | VQDLHTPFT |
| | (SEQ ID NO: 121) | (SEQ ID NO: 156) | (SEQ ID NO: 191) |
| AB344 | RSLVHGSGDNY | MAS | VQTLQTPST |
| | (SEQ ID NO: 122) | (SEQ ID NO: 157) | (SEQ ID NO: 192) |
| AB353 | QNVYTN | GAS | LQYGSSPPL |
| | (SEQ ID NO: 123) | (SEQ ID NO: 158) | (SEQ ID NO: 193) |
| AB438 | QSLGTN | GAS | QQYDTHPLS |
| | (SEQ ID NO: 124) | (SEQ ID NO: 159) | (SEQ ID NO: 194) |
| AB366 | QTISRS | DAS | QQYSSYPSL |
| | (SEQ ID NO: 125) | (SEQ ID NO: 160) | (SEQ ID NO: 195) |
| AB341 | QSLLHSNEKNY | DAS | VQDLHTPFT |
| | (SEQ ID NO: 126) | (SEQ ID NO: 161) | (SEQ ID NO: 196) |
| AB430 | QSLQQSEGRTY | DAS | MQVLRTPIT |
| | (SEQ ID NO: 127) | (SEQ ID NO: 162) | (SEQ ID NO: 197) |
| AB349 | QRIGSD | SAS | STMAIP |
| | (SEQ ID NO: 128) | (SEQ ID NO: 163) | (SEQ ID NO: 198) |
| AB449 | QGISRD | AAS | QQYVSAPPYT |
| | (SEQ ID NO: 129) | (SEQ ID NO: 164) | (SEQ ID NO: 199) |
| AB338 | QSLLHSNEKNY | DAS | VQDLHTPFT |
| | (SEQ ID NO: 130) | (SEQ ID NO: 165) | (SEQ ID NO: 200) |
| AB435 | QSIGTK | DAS | QQYGSSPPVYS |
| | (SEQ ID NO: 131) | (SEQ ID NO: 166) | (SEQ ID NO: 201) |
| AB372 | QSVLDTSTNRNY | GAS | QQSYSHPPT |
| | (SEQ ID NO: 132) | (SEQ ID NO: 167) | (SEQ ID NO: 202) |
| AB439 | NIGLRS | DTN | SSYTSSSTDV |
| | (SEQ ID NO: 133) | (SEQ ID NO: 168) | (SEQ ID NO: 203) |
| AB350 | ESVRSN | GAS | QQYHTWPPWA |
| | (SEQ ID NO: 134) | (SEQ ID NO: 169) | (SEQ ID NO: 204) |
| AB363 | QGISND | DVS | QQYDNPPS |
| | (SEQ ID NO: 135) | (SEQ ID NO: 170) | (SEQ ID NO: 205) |
| AB367 | QGIGID | DAS | RQYATFPQT |
| | (SEQ ID NO: 136) | (SEQ ID NO: 171) | (SEQ ID NO: 206) |
| AB352 | QSVSTH | DAT | LQYTNYPWT |
| | (SEQ ID NO: 137) | (SEQ ID NO: 172) | (SEQ ID NO: 207) |
| AB357 | DIRGMS | EVN | AAWDASVAGYV |
| | (SEQ ID NO: 138) | (SEQ ID NO: 173) | (SEQ ID NO: 208) |
| AB347 | QNIGHS | GAS | QQYITWPST |
| | (SEQ ID NO: 139) | (SEQ ID NO: 174) | (SEQ ID NO: 209) |
| AB346 | RSVSSD | DVS | QQYGSSLPPT |
| | (SEQ ID NO: 140) | (SEQ ID NO: 175) | (SEQ ID NO: 210) |
| AB433 | QSVRTS | DTS | QQYGNWH |
| | (SEQ ID NO: 141) | (SEQ ID NO: 176) | (SEQ ID NO: 211) |
| AB432 | QSISSSQ | GAS | QQYNDRPRT |
| | (SEQ ID NO: 142) | (SEQ ID NO: 177) | (SEQ ID NO: 212) |
| AB326 | MDIRND | DAS | QQAKSFPPT |
| | (SEQ ID NO: 143) | (SEQ ID NO: 178) | (SEQ ID NO: 213) |
| AB428 | QSLLHSSGYNF | MAT | VQDLHTLPQ |
| | (SEQ ID NO: 144) | (SEQ ID NO: 179) | (SEQ ID NO: 214) |
| AB426 | QSL | MGS | QSGVT |
| | (SEQ ID NO: 145) | (SEQ ID NO: 180) | (SEQ ID NO: 215) |
| AB345 | QSLLSSYGYHN | MGS | MQGTQAPRT |
| | (SEQ ID NO: 146) | (SEQ ID NO: 181) | (SEQ ID NO: 216) |
| AB431 | QSLLHVTGYNY | GAS | MQSTQWPHT |
| | (SEQ ID NO: 147) | (SEQ ID NO: 182) | (SEQ ID NO: 217) |
| AB330 | QDIKSD | RAS | QQYRSFPWS |
| | (SEQ ID NO: 148) | (SEQ ID NO: 183) | (SEQ ID NO: 218) |
| AB731 | SSVSY | RTY | QQRSSYPWT |
| | (SEQ ID NO: 149) | (SEQ ID NO: 184) | (SEQ ID NO: 219) |
| AB843 | SGDIGRYNY | EDI | QVWDATLDLV |
| | (SEQ ID NO: 150) | (SEQ ID NO: 185) | (SEQ ID NO: 220) |
| AB852 | QSVLDTSTNRNY | GAS | QQYETWPWT |
| | (SEQ ID NO: 151) | (SEQ ID NO: 186) | (SEQ ID NO: 221) |
| AB854 | TGSIASNY | DTT | QSTERGRSDVI |
| | (SEQ ID NO: 152) | (SEQ ID NO: 187) | (SEQ ID NO: 222) |

TABLE 8
Complementarity Determining Regions, Heavy Variable Region (Kabat)

| | Heavy Chain Variable Region CDRs | | |
|---|---|---|---|
| Name | CDR1 | CDR2 | CDR3 |
| AB448 | NAKMGVN | YVSTTGTTDYNPSLKS | AGYPDV |
| | (SEQ ID NO: 223) | (SEQ ID NO: 258) | (SEQ ID NO: 293) |
| AB465 | NARTGVN | YINPSGRIDYNPSLKS | AGYPDV |
| | (SEQ ID NO: 224) | (SEQ ID NO: 259) | (SEQ ID NO: 294) |
| AB340 | RNYMS | SISGSGDYTYYPDSVKG | SPRGPRLLGYYYGMDV |
| | (SEQ ID NO: 225) | (SEQ ID NO: 260) | (SEQ ID NO: 295) |
| AB342 | EHWMS | SIKGDASDTYYPDSVKG | DLSKQWLGIDY |
| | (SEQ ID NO: 226) | (SEQ ID NO: 261) | (SEQ ID NO: 296) |
| AB344 | NYGMS | SISKSGSSIYYPDSVKG | LPRLAWNYYYGMDV |
| | (SEQ ID NO: 227) | (SEQ ID NO: 262) | (SEQ ID NO: 297) |
| AB353 | NAKMGVN | YVSTTGTTDYNPSLKS | AGYPDV |
| | (SEQ ID NO: 228) | (SEQ ID NO: 263) | (SEQ ID NO: 298) |
| AB438 | NARLGVN | YGSVTGSTDYNPSLKS | SYLVDTAMVTR |
| | (SEQ ID NO: 229) | (SEQ ID NO: 264) | (SEQ ID NO: 299) |
| AB366 | DARIGVN | YINPSGRTDYNPSLKS | AGYPDV |
| | (SEQ ID NO: 230) | (SEQ ID NO: 265) | (SEQ ID NO: 300) |
| AB341 | NYGMS | SISKSGSSIYYPDSVKG | LPRLAWNYYYGMDV |
| | (SEQ ID NO: 231) | (SEQ ID NO: 266) | (SEQ ID NO: 301) |
| AB430 | NYPMS | STTSGSSTIYYPDSVKG | GPDYYAPLRSYMDV |
| | (SEQ ID NO: 232) | (SEQ ID NO: 267) | (SEQ ID NO: 302) |
| AB349 | NARLGVN | YGSVTGSTDYNPSLKS | SYLVDTAMVTR |
| | (SEQ ID NO: 233) | (SEQ ID NO: 268) | (SEQ ID NO: 303) |
| AB449 | NAKMGVN | YVSTTGTTDYNPSLKS | AGYPDV |
| | (SEQ ID NO: 234) | (SEQ ID NO: 269) | (SEQ ID NO: 304) |
| AB338 | RNYMS | SISGSGDYTYYPDSVKG | SPRGPRLLGYYYGMDV |
| | (SEQ ID NO: 235) | (SEQ ID NO: 270) | (SEQ ID NO: 305) |
| AB435 | DARIGVN | YINPSGRTDYNPSLKS | AGYPDV |
| | (SEQ ID NO: 236) | (SEQ ID NO: 271) | (SEQ ID NO: 306) |
| AB372 | TSGATVN | YITSSGRTVYNSALKS | TKIVGATLRDY |
| | (SEQ ID NO: 237) | (SEQ ID NO: 272) | (SEQ ID NO: 307) |
| AB439 | GYRIS | WIDPKSGRTNYAQKLQG | EVKQWLVPGRGVYWFDP |
| | (SEQ ID NO: 238) | (SEQ ID NO: 273) | (SEQ ID NO: 308) |
| AB350 | DARMGVN | YVSTTGTTDYNPSLKS | AGYPDV |
| | (SEQ ID NO: 239) | (SEQ ID NO: 274) | (SEQ ID NO: 309) |
| AB363 | TSGATVN | YITSSGRTVYNSALKS | TKIVGATLRDY |
| | (SEQ ID NO: 240) | (SEQ ID NO: 275) | (SEQ ID NO: 310) |
| AB367 | NPRMGVN | YISTSGRTDYNPSLKS | AGYPDV |
| | (SEQ ID NO: 241) | (SEQ ID NO: 276) | (SEQ ID NO: 311) |
| AB352 | TSRMGVN | YINPSGATDYNPSLKS | AGYPDV |
| | (SEQ ID NO: 242) | (SEQ ID NO: 277) | (SEQ ID NO: 312) |
| AB357 | SHYIS | GMNPITGHTIYAQKLQG | DMRYDILPGRSYYYGMDV |
| | (SEQ ID NO: 243) | (SEQ ID NO: 278) | (SEQ ID NO: 313) |
| AB347 | TSRMGVN | YINPSGATDYNPSLKS | AGYPDV |
| | (SEQ ID NO: 244) | (SEQ ID NO: 279) | (SEQ ID NO: 314) |
| AB346 | TSRMGVN | YINPSGATDYNPSLKS | AGYPDV |
| | (SEQ ID NO: 245) | (SEQ ID NO: 280) | (SEQ ID NO: 315) |
| AB433 | TSRMGVN | YINPSGATDYNPSLKS | AGYPDV |
| | (SEQ ID NO: 246) | (SEQ ID NO: 281) | (SEQ ID NO: 316) |
| AB432 | NVRMGVN | YVNPSGSTDYNPSLKS | AGYPDV |
| | (SEQ ID NO: 247) | (SEQ ID NO: 282) | (SEQ ID NO: 317) |
| AB326 | SSAIS | GIYPGSGHTIYAQKFQG | EGDIFVMDV |
| | (SEQ ID NO: 248) | (SEQ ID NO: 283) | (SEQ ID NO: 318) |
| AB428 | NHWMS | SIGPKGLGTYYPDSVKG | IRDYGDKKGGSWFDP |
| | (SEQ ID NO: 249) | (SEQ ID NO: 284) | (SEQ ID NO: 319) |
| AB426 | DHHMS | SISPSGDFTYYPDSVKG | DVSPVYY |
| | (SEQ ID NO: 250) | (SEQ ID NO: 285) | (SEQ ID NO: 320) |
| AB345 | GYWMS | SIGTAGVTYYPDSVKG | GPHGPQQRLRFDP |
| | (SEQ ID NO: 251) | (SEQ ID NO: 286) | (SEQ ID NO: 321) |
| AB431 | NHWMS | SIGPKGLGTYYPDSVKG | DVRGWRALYYYYGMDV |
| | (SEQ ID NO: 252) | (SEQ ID NO: 287) | (SEQ ID NO: 322) |
| AB330 | ATIS | WMNPKSGYTNYAQKFQG | LHGSGLAFDI |
| | (SEQ ID NO: 253) | (SEQ ID NO: 288) | (SEQ ID NO: 323) |
| AB731 | SYWMH | NINPSNGGTNYNEKFKS | TTVVAYWYFDV |
| | (SEQ ID NO: 254) | (SEQ ID NO: 289) | (SEQ ID NO: 324) |
| AB843 | HSHIH | WINPRSGATNYAQKLQG | DRGGRTNGVGAFDI |
| | (SEQ ID NO: 255) | (SEQ ID NO: 290) | (SEQ ID NO: 325) |
| AB852 | DTRWGVN | YINDRGTTDYNSALKS | TLHTREFVGMDV |
| | (SEQ ID NO: 256) | (SEQ ID NO: 291) | (SEQ ID NO: 326) |
| AB854 | SHYIS | GMNPITGHTIYAQKLQG | DMRYDILPGRSYYYGMDV |
| | (SEQ ID NO: 257) | (SEQ ID NO: 292) | (SEQ ID NO: 327) |

TABLE 9
Complementarity Determining Regions, Light Variable Region (Kabat)

| | Light Chain Variable Region CDRs | | |
|---|---|---|---|
| Name | CDR1 | CDR2 | CDR3 |
| AB448 | RASQNIGAALA | GASTREY | QQYTAYPLT |
| | (SEQ ID NO: 328) | (SEQ ID NO: 363) | (SEQ ID NO: 398) |
| AB465 | RASQTVSSHLA | DVSHRAS | QQYFTLPT |
| | (SEQ ID NO: 329) | (SEQ ID NO: 364) | (SEQ ID NO: 399) |
| AB340 | RSSRSLVHGSGHNYLH | GASTRVA | MQGTQAPRT |
| | (SEQ ID NO: 330) | (SEQ ID NO: 365) | (SEQ ID NO: 400) |
| AB342 | RSSQSLLHSNEKNYLH | DASKRAS | VQDLHTPFT |
| | (SEQ ID NO: 331) | (SEQ ID NO: 366) | (SEQ ID NO: 401) |
| AB344 | RSSRSLVHGSGDNYLH | MASNRAP | VQTLQTPST |
| | (SEQ ID NO: 332) | (SEQ ID NO: 367) | (SEQ ID NO: 402) |
| AB353 | RASQNVYTNLA | GASARAS | LQYGSSPPL |
| | (SEQ ID NO: 333) | (SEQ ID NO: 368) | (SEQ ID NO: 403) |
| AB438 | RASQSLGTNLA | GASSRPS | QQYDTHPLS |
| | (SEQ ID NO: 334) | (SEQ ID NO: 369) | (SEQ ID NO: 404) |
| AB366 | RASQTISRSLA | DASSLAG | QQYSSYPSL |
| | (SEQ ID NO: 335) | (SEQ ID NO: 370) | (SEQ ID NO: 405) |
| AB341 | RSSQSLLHSNEKNYLH | DASKRAS | VQDLHTPFT |
| | (SEQ ID NO: 336) | (SEQ ID NO: 371) | (SEQ ID NO: 406) |
| AB430 | RSSQSLQQSEGRTYLH | DASKRAS | MQVLRTPIT |
| | (SEQ ID NO: 337) | (SEQ ID NO: 372) | (SEQ ID NO: 407) |
| AB349 | RASQRIGSDLA | SASSRAP | STMAIP |
| | (SEQ ID NO: 338) | (SEQ ID NO: 373) | (SEQ ID NO: 408) |
| AB449 | RASQGISRDLA | AASTLVS | QQYVSAPPYT |
| | (SEQ ID NO: 339) | (SEQ ID NO: 374) | (SEQ ID NO: 409) |
| AB338 | RSSQSLLHSNEKNYLH | DASKRAS | VQDLHTPFT |
| | (SEQ ID NO: 340) | (SEQ ID NO: 375) | (SEQ ID NO: 410) |
| AB435 | RASQSIGTKLA | DASSRVP | QQYGSSPPVYS |
| | (SEQ ID NO: 341) | (SEQ ID NO: 376) | (SEQ ID NO: 411) |
| AB372 | KSSQSVLDTSTNRNYLA | GASIRAG | QQSYSHPPT |
| | (SEQ ID NO: 342) | (SEQ ID NO: 377) | (SEQ ID NO: 412) |
| AB439 | TGTNIGLRSVS | DTNIRSS | SSYTSSSTDV |
| | (SEQ ID NO: 343) | (SEQ ID NO: 378) | (SEQ ID NO: 413) |
| AB350 | RASESVRSNLA | GASNRAS | QQYHTWPPWA |
| | (SEQ ID NO: 344) | (SEQ ID NO: 379) | (SEQ ID NO: 414) |
| AB363 | RASQGISNDLA | DVSNRAS | QQYDNPPS |
| | (SEQ ID NO: 345) | (SEQ ID NO: 380) | (SEQ ID NO: 415) |
| AB367 | RASQGIGIDLA | DASKLQT | RQYATFPQT |
| | (SEQ ID NO: 346) | (SEQ ID NO: 381) | (SEQ ID NO: 416) |
| AB352 | RASQSVSTHLA | DATNRAT | LQYTNYPWT |
| | (SEQ ID NO: 347) | (SEQ ID NO: 382) | (SEQ ID NO: 417) |
| AB357 | TGTDIRGMSVS | EVNQRPS | AAWDASVAGYV |
| | (SEQ ID NO: 348) | (SEQ ID NO: 383) | (SEQ ID NO: 418) |
| AB347 | RASQNIGHSLA | GASIRAP | QQYITWPST |
| | (SEQ ID NO: 349) | (SEQ ID NO: 384) | (SEQ ID NO: 419) |
| AB346 | RASRSVSSDLA | DVSTRAT | QQYGSSLPPT |
| | (SEQ ID NO: 350) | (SEQ ID NO: 385) | (SEQ ID NO: 420) |
| AB433 | RASQSVRTSLA | DTSIRAA | QQYGNWH |
| | (SEQ ID NO: 351) | (SEQ ID NO: 386) | (SEQ ID NO: 421) |
| AB432 | RASQSISSSQLA | GASTRAT | QQYNDRPRT |
| | (SEQ ID NO: 352) | (SEQ ID NO: 387) | (SEQ ID NO: 422) |
| AB326 | RASMDIRNDLA | DASKLET | QQAKSFPPT |
| | (SEQ ID NO: 353) | (SEQ ID NO: 388) | (SEQ ID NO: 423) |
| AB428 | RSSQSLLHSSGYNFLH | MATNRAS | VQDLHTLPQ |
| | (SEQ ID NO: 354) | (SEQ ID NO: 389) | (SEQ ID NO: 424) |
| AB426 | RSSQSLLH | MGSQRAA | QSGVT |
| | (SEQ ID NO: 355) | (SEQ ID NO: 390) | (SEQ ID NO: 425) |
| AB345 | RSSQSLLSSYGYHNLH | MGSIRAP | MQGTQAPRT |
| | (SEQ ID NO: 356) | (SEQ ID NO: 391) | (SEQ ID NO: 426) |
| AB431 | RSSQSLLHVTGYNYLH | GASSRAA | MQSTQWPHT |
| | (SEQ ID NO: 357) | (SEQ ID NO: 392) | (SEQ ID NO: 427) |
| AB330 | RASQDIKSDLA | RASARES | QQYRSFPWS |
| | (SEQ ID NO: 358) | (SEQ ID NO: 393) | (SEQ ID NO: 428) |
| AB731 | SASSSVSYMY | RTYNLAS | QQRSSYPWT |
| | (SEQ ID NO: 359) | (SEQ ID NO: 394) | (SEQ ID NO: 429) |
| AB843 | TGTSGDIGRYNYVS | EDIERPS | QVWDATLDLV |
| | (SEQ ID NO: 360) | (SEQ ID NO: 395) | (SEQ ID NO: 430) |
| AB852 | KSSQSVLDTSTNRNYLA | GASIRAG | QQYETWPWT |
| | (SEQ ID NO: 361) | (SEQ ID NO: 396) | (SEQ ID NO: 431) |
| AB854 | TGTTGSIASNYVS | DTTNRPP | QSTERGRSDVI |
| | (SEQ ID NO: 362) | (SEQ ID NO: 397) | (SEQ ID NO: 432) |

TABLE 10
Illustrative Immunoglobulin Sequences-
Heavy Chain Variable Region

| Name | SEQ ID | Heavy Chain Variable Region |
|---|---|---|
| AB448 | 433 | QVQLQESGPGLVKPSQTLSLTCTVSGLSLS |
| | | NAKMGVNWIRQPPGKGLEWIGYVSTTGTTD |
| | | YNPSLKSRVTMSVDTSKNQFSLKVNSVTAA |
| | | DTAVYYCARAGYPDVWGKGTTVTVSS |
| AB465 | 435 | QVQLQESGPGLVKPSQTLSLTCTVSGASVS |
| | | NARTGVNWIRQPPGKGLEWIGYINPSGRID |
| | | YNPSLKSRVTMSVDTSKNQFSLKVNSVTAA |
| | | DTAVYYCARAGYPDVWGKGTTVTVSS |
| AB340 | 437 | EVQLVESGGGLVQPGGSLRLSCAASGFTAS |
| | | RNYMSWVRQAPGKGLELVASISGSGDYTYY |
| | | PDSVKGRFTISRDNAKNSLYLQMNSLRAED |
| | | TAVYYCARSPRGPRLLGYYYGMDVWGQGTT |
| | | VTVSS |
| AB342 | 439 | EVQLVESGGGLVQPGGSLRLSCAASGFDFG |
| | | EHWMSWVRQAPGKGLELVASIKGDASDTYY |
| | | PDSVKGRFTISRDNAKNSLYLQMNSLRAED |
| | | TAVYYCVKDLSKQWLGIDYWGQGTLVTVSS |
| AB344 | 441 | EVQLVESGGGLVQPGGSLRLSCAASGYTFS |
| | | NYGMSWVRQAPGKGLELVASISKSGSSIYY |
| | | PDSVKGRFTISRDNAKNSLYLQMNSLRAED |
| | | TAVYYCARLPRLAWNYYYGMDVWGQGTTVT |
| | | VSS |
| AB353 | 443 | QVQLQESGPGLVKPSQTLSLTCTVSGLSLS |
| | | NAKMGVNWIRQPPGKGLEWIGYVSTTGTTD |
| | | YNPSLKSRVTMSVDTSKNQFSLKVNSVTAA |
| | | DTAVYYCARAGYPDVWGKGTTVTVSS |
| AB438 | 445 | QVQLQESGPGLVKPSQTLSLTCTVSGVSLS |
| | | NARLGVNWIRQPPGKGLEWIGYGSVTGSTD |
| | | YNPSLKSRVTMSVDTSKNQFSLKVNSVTAA |
| | | DTAVYYCTTSYLVDTAMVTRWGQGTLVTVS |
| | | S |
| AB366 | 447 | QVQLQESGPGLVKPSQTLSLTCTVSGYSLT |
| | | DARIGVNWIRQPPGKGLEWIGYINPSGRTD |
| | | YNPSLKSRVTMSVDTSKNQFSLKVNSVTAA |
| | | DTAVYYCARAGYPDVWGKGTTVTVSS |
| AB341 | 449 | EVQLVESGGGLVQPGGSLRLSCAASGYTFS |
| | | NYGMSWVRQAPGKGLELVASISKSGSSIYY |
| | | PDSVKGRFTISRDNAKNSLYLQMNSLRAED |
| | | TAVYYCARLPRLAWNYYYGMDVWGQGTTVT |
| | | VSS |
| AB430 | 451 | EVQLVESGGGLVQPGGSLRLSCAASGFTLS |
| | | NYPMSWVRQAPGKGLELVASTTSGSSTIYY |
| | | PDSVKGRFTISRDNAKNSLYLQMNSLRAED |
| | | TAVYYCARGPDYYAPLRSYMDVWGKGTTVT |
| | | VSS |
| AB349 | 453 | QVQLQESGPGLVKPSQTLSLTCTVSGVSLS |
| | | NARLGVNWIRQPPGKGLEWIGYGSVTGSTD |
| | | YNPSLKSRVTMSVDTSKNQFSLKVNSVTAA |
| | | DTAVYYCTTSYLVDTAMVTRWGQGTLVTVS |
| | | S |
| AB449 | 455 | QVQLQESGPGLVKPSQTLSLTCTVSGLSLS |
| | | NAKMGVNWIRQPPGKGLEWIGYVSTTGTTD |
| | | YNPSLKSRVTMSVDTSKNQFSLKVNSVTAA |
| | | DTAVYYCARAGYPDVWGKGTTVTVSS |
| AB338 | 457 | EVQLVESGGGLVQPGGSLRLSCAASGFTAS |
| | | RNYMSWVRQAPGKGLELVASISGSGDYTYY |
| | | PDSVKGRFTISRDNAKNSLYLQMNSLRAED |
| | | TAVYYCARSPRGPRLLGYYYGMDVWGQGTT |
| | | VTVSS |
| AB435 | 459 | QVQLQESGPGLVKPSQTLSLTCTVSGYSLT |
| | | DARIGVNWIRQPPGKGLEWIGYINPSGRTD |
| | | YNPSLKSRVTMSVDTSKNQFSLKVNSVTAA |
| | | DTAVYYCARAGYPDVWGKGTTVTVSS |
| AB372 | 461 | QVTLRESGPALVKPTQTLTLTCTVSGFSLS |
| | | TSGATVNWIRQPPGKALEWIGYITSSGRTV |
| | | YNSALKSRLTISKDTSKNQVVLTMTNMDPV |
| | | DTATYYCTTTKIVGATLRDYWGQGTLVTVS |
| | | S |
| AB439 | 463 | EVQLVQSGAEVKKPGASVKVSCKASGYTYT |
| | | GYRISWVRQAPGQGLEWMGWIDPKSGRTNY |
| | | AQKLQGRGTMTTDPSTSTAYMELRSLRSDD |
| | | TAVYYCAREVKQWLVPGRGVYWFDPWGQGT |
| | | LVTVSS |
| AB350 | 465 | QVQLQESGPGLVKPSQTLSLTCTVSGVSLS |
| | | DARMGVNWIRQPPGKGLEWIGYVSTTGTTD |
| | | YNPSLKSRVTMSVDTSKNQFSLKVNSVTAA |
| | | DTAVYYCARAGYPDVWGKGTLVTVSS |
| AB363 | 467 | QVTLRESGPALVKPTQTLTLTCTVSGFSLS |
| | | TSGATVNWIRQPPGKALEWIGYITSSGRTV |
| | | YNSALKSRLTISKDTSKNQVVLTMTNMDPV |
| | | DTATYYCTTTKIVGATLRDYWGQGTLVTVS |
| | | S |
| AB367 | 469 | QVTLRESGPALVKPTQTLTLTCTVSGFTLS |
| | | NPRMGVNWIRQPPGKGLEWIGYISTSGRTD |
| | | YNPSLKSRVTMSVDTSKNQFSLKVNSVTAA |
| | | DTAVYYCARAGYPDVWGKGTTVTVSS |
| AB352 | 471 | QVQLQESGPGLVKPSQTLSLTCTVSGFPLS |
| | | TSRMGVNWIRQPPGKGLEWIGYINPSGATD |
| | | YNPSLKSRVTMSVDTSKNQFSLKVNSVTAA |
| | | DTAVYYCARAGYPDVWGKGTTVTVSS |
| AB357 | 473 | EVQLVQSGAEVKKPGASVKVSCKASGYTFP |
| | | SHYISWVRQAPGQGLEWMGGMNPITGHTIY |
| | | AQKLQGRGTMTTDPSTSTAYMELRSLRSDD |
| | | TAVYYCARDMRYDILPGRSYYYGMDVWGQG |
| | | TTVTVSS |
| AB347 | 475 | QVQLQESGPGLVKPSQTLSLTCTVSGFPLS |
| | | TSRMGVNWIRQPPGKGLEWIGYINPSGATD |
| | | YNPSLKSRVTMSVDTSKNQFSLKVNSVTAA |
| | | DTAVYYCARAGYPDVWGKGTTVTVSS |
| AB346 | 477 | QVQLQESGPGLVKPSQTLSLTCTVSGFPLS |
| | | TSRMGVNWIRQPPGKGLEWIGYINPSGATD |
| | | YNPSLKSRVTMSVDTSKNQFSLKVNSVTAA |
| | | DTAVYYCARAGYPDVWGKGTTVTVSS |
| AB433 | 479 | QVQLQESGPGLVKPSQTLSLTCTVSGFPLS |
| | | TSRMGVNWIRQPPGKGLEWIGYINPSGATD |
| | | YNPSLKSRVTMSVDTSKNQFSLKVNSVTAA |
| | | DTAVYYCARAGYPDVWGKGTTVTVSS |
| AB432 | 481 | QVQLQESGPGLVKPSQTLSLTCTVSGVSLS |
| | | NVRMGVNWIRQPPGKGLEWIGYVNPSGSTD |
| | | YNPSLKSRVTMSVDTSKNQFSLKVNSVTAA |
| | | DTAVYYCARAGYPDVWGKGTLVTVSS |
| AB326 | 483 | QVQLVQSGAEVKKPGASVKVSCKVSGYTFT |
| | | SSAISWVRQAPGKGLEWMGGIYPGSGHTIY |
| | | AQKFQGRVTMTEDTSTDTAYMELSSLKSED |
| | | TAVYYCAREGDIFVMDVWGLGTAVTVSS |
| AB428 | 485 | EVQLVESGGGLVQPGGSLRLSCAASGFTSS |
| | | NHWMSWVRQAPGKGLELVASIGPKGLGTYY |
| | | PDSVKGRFTISRDNAKNSLYLQMNSLRAED |
| | | TAVYYCASIRDYGDKKGGSWFDPWGQGTLV |
| | | TVSS |
| AB426 | 487 | EVQLVESGGGLVQPGGSLRLSCAASGFTFS |
| | | DHHMSWVRQAPGKGLELVASISPSGDFTYY |
| | | PDSVKGRFTISRDNAKNSLYLQMNSLRAED |
| | | TAVYYCARDVSPVYYWGQGALVTVSS |
| AB345 | 489 | EVQLVESGGGLVQPGGSLRLSCAASGFTSS |
| | | GYWMSWFRQAPGKGLELVASIGTAGVTYYP |
| | | DSVKGRFTISRDNAKNSLYLQMNSLRAEDT |
| | | AVYYCARGPHGPQQRLRFDPWGQGTLVTVS |
| | | S |
| AB431 | 491 | EVQLVESGGGLVQPGGSLRLSCAASGFTSS |
| | | NHWMSWVRQAPGKGLELVASIGPKGLGTYY |
| | | PDSVKGRFTISRDNAKNSLYLQMNSLRAED |
| | | TAVYYCARDVRGWRALYYYYGMDVWGQGTT |
| | | VTVSS |
| AB330 | 493 | QVQLVQSGAEVKKPGASVKVSCKVSGYPSP |
| | | ATISWVRQAPGKGLEWMGWMNPKSGYTNYA |
| | | QKFQGRVTMTEDTSTDTAYMELSSLKSEDT |
| | | AVYYCATLHGSGLAFDIWGQGTMVTVSS |
| AB731 | 495 | QVQLQQPGTELVKPGASVKLSCKASGYTFT |
| | | SYWMHWVKQRPGQGLEWIGNINPSNGGTNY |
| | | NEKFKSKATLTVDKSSSTAYMQLSSLTSED |
| | | SAVYYCSITTWAYWYFDVWGTGTTVTVSS |
| AB843 | 497 | EVQLVQSGAEVKKPGASVKVSCKASGDTSS |
| | | HSHIHWVRQAPGQGLEWMGWINPRSGATNY |
| | | AQKLQGRGTMTTDPSTSTAYMELRSLRSDD |
| | | TAVYYCARDRGGRTNGVGAFDIWGQGTMVT |
| | | VSS |
| AB852 | 499 | QVTLRESGPALVKPTQTLTLTCTVSGFSLS |
| | | DTRWGVNWIRQPPGKALEWIGYINDRGTTD |
| | | YNSALKSRLTISKDTSKNQVVLTMTNMDPV |
| | | DTATYYCAHTLHTREFVGMDVWGQGTTVTV |
| | | SS |
| AB854 | 501 | EVQLVQSGAEVKKPGASVKVSCKASGYTFP |
| | | SHYISWVRQAPGQGLEWMGGMNPITGHTIY |
| | | AQKLQGRGTMTTDPSTSTAYMELRSLRSDD |
| | | TAVYYCARDMRYDILPGRSYYYGMDVWGQG |
| | | TTVTVSS |

TABLE 11
Illustrative Immunoglobulin Sequences-
Light Chain Variable Region

| Name | SEQ ID | Light Chain Variable Region |
|---|---|---|
| AB448 | 434 | DIQMTQSPSSVSASVGDRVTITCRASQNIG |
| | | AALAWYQQKPGKAPKLLIYGASTREYGVPS |
| | | RFSGSGSGTDFTLTISSLQPEDFANYYCQQ |
| | | YTAYPLTFGGGTKVEIK |
| AB465 | 436 | EIVMTQSPATLSLSPGERATLSCRASQTVS |
| | | SHLAWYQQKPGQAPRLLIYDVSHRASGIPA |
| | | RFSGSGSGTDFTLTISSLEPEDFAVYYCQQ |
| | | YFTLPTFGGGTKVEIK |
| AB340 | 438 | DIVMTQSPLSLPVTPGEPASISCRSSRSLV |
| | | HGSGHNYLHWYLQKPGQSPQLLIYGASTRV |
| | | AGVPDRFSGSGSGTDFTLKISRVEAEDVGV |
| | | YYCMQGTQAPRTFGGGTKVEIK |
| AB342 | 440 | DIVMTQSPLSLPVTPGEPASISCRSSQSLL |
| | | HSNEKNYLHWYLQKPGQSPQLLIYDASKRA |
| | | SGVPDRFSGSGSGTDFTLKISRVEAEDVGV |
| | | YYCVQDLHTPFTFGGGTKVEIK |
| AB344 | 442 | DIVMTQSPLSLPVTPGEPASISCRSSRSLV |
| | | HGSGDNYLHWYLQKPGQSPQLLIYMASNRA |
| | | PGVPDRFSGSGSGTDFTLKISRVEAEDVGV |
| | | YYCVQTLQTPSTFGGGTKVEIK |
| AB353 | 444 | EIVMTQSPATLSLSPGERATLSCRASQNVY |
| | | TNLAWYQQKPGQAPRLLIYGASARASGIPA |
| | | RFSGSGSGTDFTLTISSLEPEDFAVYYCLQ |
| | | YGSSPPLFGGGTKVEIK |
| AB438 | 446 | EIVMTQSPATLSLSPGERATLSCRASQSLG |
| | | TNLAWYQQKPGQAPRLLIYGASSRPSGIPA |
| | | RFSGSGSGTDFTLTISSLEPEDFAVYYCQQ |
| | | YDTHPLSFGGGTKVEIK |
| AB366 | 448 | DIQMTQSPSSVSASVGDRVTITCRASQTIS |
| | | RSLAWYQQKPGKAPKLLIYDASSLAGGVPS |
| | | RFSGSGSGTDFTLTISSLQPEDFANYYCQQ |
| | | YSSYPSLFGGGTKVEIK |
| AB341 | 450 | DIVMTQSPLSLPVTPGEPASISCRSSQSLL |
| | | HSNEKNYLHWYLQKPGQSPQLLIYDASKRA |
| | | SGVPDRFSGSGSGTDFTLKISRVEAEDVGV |
| | | YYCVQDLHTPFTFGGGTKVEIK |
| AB430 | 452 | DIVMTQSPLSLPVTPGEPASISCRSSQSLQ |
| | | QSEGRTYLHWYLQKPGQSPQLLIYDASKRA |
| | | SGVPDRFSGSGSGTDFTLKISRVEAEDVGV |
| | | YYCMQVLRTPITFGGGTKVEIK |
| AB349 | 454 | EIVMTQSPATLSLSPGERATLSCRASQRIG |
| | | SDLAWYQQKPGQAPRLLIYSASSRAPGIPA |
| | | RFSGSGSGTDFTLTISSLEPEDFAVYYCST |
| | | MAIPFGGGTKVEIK |
| AB449 | 456 | DIQMTQSPSSVSASVGDRVTITCRASQGIS |
| | | RDLAWYQQKPGKAPKLLIYAASTLVSGVPS |
| | | RFSGSGSGTDFTLTISSLQPEDFANYYCQQ |
| | | YVSAPPYTFGGGTKVEIK |
| AB338 | 458 | DIVMTQSPLSLPVTPGEPASISCRSSQSLL |
| | | HSNEKNYLHWYLQKPGQSPQLLIYDASKRA |
| | | SGVPDRFSGSGSGTDFTLKISRVEAEDVGV |
| | | YYCVQDLHTPFTFGGGTKVEIK |
| AB435 | 460 | EIVMTQSPATLSLSPGERATLSCRASQSIG |
| | | TKLAWYQQKPGQAPRLLIYDASSRVPGIPA |
| | | RFSGSGSGTDFTLTISSLEPEDFAVYYCQQ |
| | | YGSSPPVYSFGGGTKVEIK |
| AB372 | 462 | DIVMTQSPDSLAVSLGERATINCKSSQSVL |
| | | DTSTNRNYLAWYQQKPGQPPKLLIYGASIR |
| | | AGGVPDRFSGSGSGTDFTLTISSLQAEDVA |
| | | VYYCQQSYSHPPTFGGGTKVEIK |
| AB439 | 464 | ESALTQPASVSGSPGQSITISCTGTNIGLR |
| | | SVSWYQQHPGKAPKLMIYDTNIRSSGVSNR |
| | | FSGSKSGNTASLTISGLQAEDEADYYCSSY |
| | | TSSSTDVFGGGTKLTVL |
| AB350 | 466 | EIVMTQSPATLSLSPGERATLSCRASESVR |
| | | SNLAWYQQKPGQAPRLLIYGASNRASGIPA |
| | | RFSGSGSGTDFTLTISSLEPEDFAVYYCQQ |
| | | YHTWPPWAFGGGTKVEIK |
| AB363 | 468 | DIQMTQSPSSVSASVGDRVTITCRASQGIS |
| | | NDLAWYQQKPGKAPKLLIYDVSNRASGVPS |
| | | RFSGSGSGTDFTLTISSLQPEDFANYYCQQ |
| | | YDNPPSFGGGTKVEIK |
| AB367 | 470 | DIQMTQSPSSVSASVGDRVTITCRASQGIG |
| | | IDLAWYQQKPGKAPKLLIYDASKLQTGVPS |
| | | RFSGSGSGTDFTLTISSLQPEDFANYYCRQ |
| | | YATFPQTFGGGTKVEIK |
| AB352 | 472 | EIVMTQSPATLSLSPGERATLSCRASQSVS |
| | | THLAWYQQKPGQAPRLLIYDATNRATGIPA |
| | | RFSGSGSGTDFTLTISSLEPEDFAVYYCLQ |
| | | YTNYPWTFGGGTKVEIK |
| AB357 | 474 | ESALTQPASVSGSPGQSITISCTGTDIRGM |
| | | SVSWYQQHPGKAPKLMIYEVNQRPSGVSNR |
| | | FSGSKSGNTASLTISGLQAEDEADYYCAAW |
| | | DASVAGYVFGGGTKLTVL |
| AB347 | 476 | EIVMTQSPATLSLSPGERATLSCRASQNIG |
| | | HSLAWYQQKPGQAPRLLIYGASIRAPGIPA |
| | | RFSGSGSGTDFTLTISSLEPEDFAVYYCQQ |
| | | YITWPSTFGGGTKVEIK |
| AB346 | 478 | EIVMTQSPATLSLSPGERATLSCRASRSVS |
| | | SDLAWYQQKPGQAPRLLIYDVSTRATGIPA |
| | | RFSGSGSGTDFTLTISSLEPEDFAVYYCQQ |
| | | YGSSLPPTFGGGTKVEIK |
| AB433 | 480 | EIVMTQSPATLSLSPGERATLSCRASQSVR |
| | | TSLAWYQQKPGQAPRLLIYDTSIRAAGIPA |
| | | RFSGSGSGTDFTLTISSLEPEDFAVYYCQQ |
| | | YGNWHFGGGTKVEIK |
| AB432 | 482 | EIVMTQSPATLSLSPGERATLSCRASQSIS |
| | | SSQLAWYQQKPGQAPRLLIYGASTRATGIP |
| | | ARFSGSGSGTDFTLTISSLEPEDFAVYYCQ |
| | | QYNDRPRTFGGGTKVEIK |
| AB326 | 484 | DIQMTQSPSSVSASVGDRVTITCRASMDIR |
| | | NDLAWYQQKPGKAPKLLIYDASKLETGVPS |
| | | RFSGSGSGTDFTLTISSLQPEDFANYYCQQ |
| | | AKSFPPTFGGGTKVEIK |
| AB428 | 486 | DIVMTQSPLSLPVTPGEPASISCRSSQSLL |
| | | HSSGYNFLHWYLQKPGQSPQLLIYMATNRA |
| | | SGVPDRFSGSGSGTDFTLKISRVEAEDVGV |
| | | YYCVQDLHTLPQFGGGTKVEIK |
| AB426 | 488 | DIVMTQSPLSLPVTPGEPASISCRSSQSLL |
| | | HWYLQKPGQSPQLLIYMGSQRAAGVPDRFS |
| | | GSGSGTDFTLKISRVEAEDVGVYYCQSGVT |
| | | FGGGTKVEIK |
| AB345 | 490 | DIVMTQSPLSLPVTPGEPASISCRSSQSLL |
| | | SSYGYHNLHWYLQKPGQSPQLLIYMGSIRA |
| | | PGVPDRFSGSGSGTDFTLKISRVEAEDVGV |
| | | YYCMQGTQAPRTFGGGTKVEIK |
| AB431 | 492 | DIVMTQSPLSLPVTPGEPASISCRSSQSLL |
| | | HVTGYNYLHWYLQKPGQSPQLLIYGASSRA |
| | | AGVPDRFSGSGSGTDFTLKISRVEAEDVGV |
| | | YYCMQSTQWPHTFGGGTKVEIK |
| AB330 | 494 | DIQMTQSPSSVSASVGDRVTITCRASQDIK |
| | | SDLAWYQQKPGKAPKLLIYRASARESGVPS |
| | | RFSGSGSGTDFTLTISSLQPEDFANYYCQQ |
| | | YRSFPWSFGGGTKVEIK |
| AB731 | 496 | ENVLTQSPAIMSASPGEKVTISCSASSSVS |
| | | YMYWYQQKPGSSPKPWIFRTYNLASGVPAR |
| | | FSGSGSGTSYSLTISNMEAEDAATYYCQQR |
| | | SSYPWTFGGGTKLEIK |
| AB843 | 498 | ESALTQPASVSGSPGQSITISCTGTSGDIG |
| | | RYNYVSWYQQHPGKAPKLMIYEDIERPSGV |
| | | SNRFSGSKSGNTASLTISGLQAEDEADYYC |
| | | QVWDATLDLVFGGGTKLTVL |
| AB852 | 500 | DIVMTQSPDSLAVSLGERATINCKSSQSVL |
| | | DTSTNRNYLAWYQQKPGQPPKLLIYGASIR |
| | | AGGVPDRFSGSGSGTDFTLTISSLQAEDVA |
| | | VYYCQQYETWPWTFGGGTKVEIK |
| AB854 | 502 | ESALTQPASVSGSPGQSITISCTGTTGSIA |
| | | SNYVSWYQQHPGKAPKLMIYDTTNRPPGVS |
| | | NRFSGSKSGNTASLTISGLQAEDEADYYCQ |
| | | STERGRSDVIFGGGTKLTVL |

1. A method for treating a solid tumor comprising cancer-associated fibroblasts (CAF) in a subject in need thereof, the method comprising simultaneously or sequentially administering to the subject a therapeutically effective dose of at least one TGF-β inhibitor and at least one Activin inhibitor.

2. The method of claim 1, wherein the tumor is from a cancer selected from the group consisting of breast cancer, colon cancer, and pancreatic cancer.

3. The method of claim 1, wherein the TGF-β inhibitor and the Activin inhibitor are each independently selected from a peptidic inhibitor and an antibody inhibitor.

4. The method of claim 3, wherein the TGF-β inhibitor is an antibody inhibitor selected from the group consisting of anti-TGF-β antibody clone 1D11, Fresolimumab, and LY3022859; and the Activin inhibitor is an antibody inhibitor selected from the group consisting of Garetosmab, and AMGA1, or the peptidic inhibitor Follistatin Fc.

5. The method of claim 3, wherein the TGF-β inhibitor is an antibody inhibitor selected from the group consisting of anti-TGF-β antibody clone 1D11, Fresolimumab, and LY3022859; and the Activin inhibitor is an antibody inhibitor comprising a heavy chain variable region/light chain variable region (HCVR/LCVR) sequence pair selected from the group consisting of SEQ ID NOs: 433/434, 435/436, 437/438, 439/440, 441/442, 443/444, 445/446, 447/448, 449/450, 451/452, 453/454, 455/456, 457/458, 459/460, 461/462, 463/464, 465/466, 467/468, 469/470, 471/472, 473/474, 475/476, 477/478, 479/480, 481/482, 483/484, 485/486, 487/488, 489/490, 491/492, 493/494, 495/496, 497/498, 499/500, and 501/502; or
the Activin inhibitor is an antibody inhibitor comprising the six complementarity determining regions (CDRs) of a HCVR/LCVR sequence pair selected from the group consisting of SEQ ID NOs: 433/434, 435/436, 437/438, 439/440, 441/442, 443/444, 445/446, 447/448, 449/450, 451/452, 453/454, 455/456, 457/458, 459/460, 461/462, 463/464, 465/466, 467/468, 469/470, 471/472, 473/474, 475/476, 477/478, 479/480, 481/482, 483/484, 485/486, 487/488, 489/490, 491/492, 493/494, 495/496, 497/498, 499/500, and 501/502.

6. The method of claim 1, wherein the TGF-β inhibitor targets TGF-β 1.

7. The method of claim 5, wherein the TGF-β inhibitor further targets TGF-β3.

8. The method of claim 1, wherein the Activin inhibitor is an anti-activin A antibody.

9. The method of claim 8, wherein the Activin inhibitor also inhibits activin B.

10. The method of claim 1, wherein the administering is systemic administration.

11. The method of claim 10, wherein the therapeutically effective doses of the TGF-β inhibitor and the Activin inhibitor when given in combination are lower than the therapeutically effective dose of either inhibitor given individually.

12. The method of claim 10, wherein the therapeutically effective dose of the Activin inhibitor is higher than the therapeutically effective dose of the TGF-β inhibitor.

13. The method of claim 1, wherein the composition further comprises an immune checkpoint inhibitor.

14. The method of claim 13, wherein the immune checkpoint inhibitor is a PD-1 checkpoint inhibitor.

15. A method for inhibiting the transformation of normal tissue fibroblasts into cancer associated fibroblasts (CAFs) in a patient in need thereof, the method comprising simultaneously or sequentially administering to the patient at least one TGF-β inhibitor and at least one Activin inhibitor.

16. A method of inhibiting growth of a solid tumor enriched in cancer-associated fibroblasts in a patient in need thereof, the method comprising simultaneously or sequentially administering to the patient at least one TGF-β inhibitor and at least one an Activin inhibitor.

17. The method of claim 16, wherein the tumor is from a cancer selected from the group consisting of breast cancer, colon cancer, and pancreatic cancer.

18. The method of claim 15, wherein the TGF-β inhibitor and the Activin inhibitor are each independently selected from a peptidic inhibitor and an antibody inhibitor.

19. The method of claim 18, wherein the TGF-β inhibitor is an antibody inhibitor selected from the group consisting of anti-TGF-β antibody clone 1D11, Fresolimumab, and LY3022859; and the Activin inhibitor is an antibody inhibitor selected from the group consisting of Garetosmab, and AMGA1, or the peptidic inhibitor Follistatin Fc.

20. The method of claim 18, wherein the TGF-β inhibitor is an antibody inhibitor selected from the group consisting of anti-TGF-β antibody clone 1D11, Fresolimumab, and LY3022859; and the Activin inhibitor is an antibody inhibitor comprising a heavy chain variable region/light chain variable region (HCVR/LCVR) sequence pair selected from the group consisting of SEQ ID NOs: 433/434, 435/436, 437/438, 439/440, 441/442, 443/444, 445/446, 447/448, 449/450, 451/452, 453/454, 455/456, 457/458, 459/460, 461/462, 463/464, 465/466, 467/468, 469/470, 471/472, 473/474, 475/476, 477/478, 479/480, 481/482, 483/484, 485/486, 487/488, 489/490, 491/492, 493/494, 495/496, 497/498, 499/500, and 501/502; or
the Activin inhibitor is an antibody inhibitor comprising the six complementarity determining regions (CDRs) of a HCVR/LCVR sequence pair selected from the group consisting of SEQ ID NOs: 433/434, 435/436, 437/438, 439/440, 441/442, 443/444, 445/446, 447/448, 449/450, 451/452, 453/454, 455/456, 457/458, 459/460, 461/462, 463/464, 465/466, 467/468, 469/470, 471/472, 473/474, 475/476, 477/478, 479/480, 481/482, 483/484, 485/486, 487/488, 489/490, 491/492, 493/494, 495/496, 497/498, 499/500, and 501/502

21. The method of claim 15 wherein the administering is systemic or intra-tumoral injection.

22. The method of claim 15, wherein the composition further comprises an immune checkpoint inhibitor.

23. The method of claim 22, wherein the immune checkpoint inhibitor is a PD-1 checkpoint inhibitor.

24. A method for treating a fibrotic disease in a subject in need thereof, the method comprising simultaneously or sequentially administering to the subject a therapeutically effective dose of at least one TGF-β inhibitor and at least one Activin inhibitor.

25. The method of claim 24, wherein the fibrotic disease is selected from the group consisting of Pulmonary Fibrosis (PF), Idiopathic Pulmonary Fibrosis (IPF), Liver Cirrhosis, Non-alcoholic steric hepatitis (NASH), Pulmonary Arterial Hypertension (PAH), Renal Fibrosis, Chronic Kidney Disease (CKD), Chronic Dermal Fibrosis, Scleroderma, Hypertrophic Scar, Cardiac Fibrosis, Hypertrophic Cardiomyopathy.

26. A pharmaceutical composition comprising at least one TGF-β inhibitor and at least one Activin inhibitor in a pharmaceutically acceptable carrier.

27. The pharmaceutical composition of claim 26, wherein the TGF-β inhibitor and the Activin inhibitor are each independently selected from a peptidic inhibitor and an antibody inhibitor.

28. The pharmaceutical composition of claim 27, wherein the TGF-β inhibitor is an antibody inhibitor selected from the group consisting of anti-TGF-β antibody clone 1D11, Fresolimumab, and LY3022859; and the Activin inhibitor is an antibody inhibitor selected from the group consisting of Garetosmab, and AMGA1, or the peptidic inhibitor Follistatin Fc.

29. The pharmaceutical composition of claim 27, wherein the TGF-β inhibitor is an antibody inhibitor selected from the group consisting of anti-TGF-β antibody clone 1D11, Fresolimumab, and LY3022859; and the Activin inhibitor is an antibody inhibitor comprising a heavy chain variable region/light chain variable region (HCVR/LCVR) sequence pair selected from the group consisting of SEQ ID NOs: 433/434, 435/436, 437/438, 439/440, 441/442, 443/444, 445/446, 447/448, 449/450, 451/452, 453/454, 455/456, 457/458, 459/460, 461/462, 463/464, 465/466, 467/468, 469/470, 471/472, 473/474, 475/476, 477/478, 479/480, 481/482, 483/484, 485/486, 487/488, 489/490, 491/492, 493/494, 495/496, 497/498, 499/500, and 501/502; or
the Activin inhibitor is an antibody inhibitor comprising the six complementarity determining regions (CDRs) of a HCVR/LCVR sequence pair selected from the group consisting of SEQ ID NOs: 433/434, 435/436, 437/438, 439/440, 441/442, 443/444, 445/446, 447/448, 449/450, 451/452, 453/454, 455/456, 457/458, 459/460, 461/462, 463/464, 465/466, 467/468, 469/470, 471/472, 473/474, 475/476, 477/478, 479/480, 481/482, 483/484, 485/486, 487/488, 489/490, 491/492, 493/494, 495/496, 497/498, 499/500, and 501/502

30. The pharmaceutical composition of claim 26, further comprising an immune checkpoint inhibitor.

31. The pharmaceutical composition of claim 30, wherein the immune checkpoint inhibitor is a PD-1 checkpoint inhibitor.

32-38. (canceled)
